(19) United States
(12) Patent Application Publication
(10) Pub. No.: US 2003/0186967 A1
(43) Pub. Date: Oct. 2, 2003

(54) Acylresorcinol derivatives are selective vitronectin receptor inhibitors

(75) Inventors: Kenneth Lewis Kees, Glenmoore, PA (US); Lloyd M. Garrick, Norristown, PA (US); Ariamala Gopalsamy, Mahwah, NJ (US)

Correspondence Address: Steven R. Eck, Five Giralda Farms, Madison, NJ, 07940, US

(73) Assignee: American Home Products Corporation, Madison, NJ (US)

(21) Appl. No.: 10/068,711

(22) Filed: Feb. 6, 2002

Related U.S. Application Data
(63) Continuation-in-part of application No. 09/291,558, filed on Apr. 14, 1999.
(60) Provisional application No. 60/081,662, filed on Apr. 14, 1998.

(51) Int. Cl. A61K 31/55; A61K 31/505; A61K 31/4168; A61K 31/24

(52) U.S. Cl. 514/217.11; 514/275; 514/269; 514/352; 514/398; 514/534; 540/605; 544/317; 544/330; 546/304; 548/326.5; 560/41

(57) ABSTRACT

Compounds of the formula

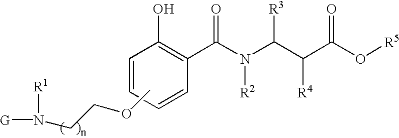

wherein R¹, R², R³, R⁴, R⁵ and n are as defined in the specification are useful in the treatment of various disorders including, but not limited to, cancer, angiogenesis, restenosis, inflammation, bone diseases, and as antiviral agents.

This is a continuation-in-part of copending application Ser. No. 09/291,558 filed on Apr. 14, 1999, which claims the benefit of U.S. Provisional Application No. 60/081,662 filed Apr. 14, 1998, the entire disclosure of which is hereby incorporated by reference.

### BACKGROUND OF INVENTION

The integrin αᵥβ₃ has been shown to mediate the invasion of cancerous melanoma cells into healthy tissue (Sefton et al., Proc. Natl. Acad. Sci, USA, 1992, 89, 1557-1561) and to protect these cells against natural cell death cycle (apoptosis) (Montgomery et al., Proc. Natl. Acad. Sci. USA, 1994, 91, 8856-8860). Vitronectin receptor (αᵥβ₃) antagonists have been shown to inhibit the growth of various solid tumors of human origin (Brooks et al., Cell, 1994, 79, 1157-1164). More recently, αᵥβ₃ has been shown to be involved in liver metastasis (Yun et al., Cancer Res., 1996, 56, 3103-3111).

Although angiogenesis is an important and natural process in growth and wound healing, it is now appreciated that a variety of clinically relevant conditions are pathologically related to these processes, and that the integrin αᵥβ₃ is involved. For example, αᵥβ₃ was shown to be expressed on human wound tissue but not on normal skin (Brooks, et al., Science, 1994, 264, 569-571) and is preferentially expressed on angiogenic blood vessels, such as those feeding a growing/invading tumor. It has also been shown that antagonists of αᵥβ₃ promote tumor regression by inducing apoptosis of the tumor cells (Brooks et al., Cell, 1994, 79, 1157-1164). This process of neovascularization which is critical for tumor growth and metastasis, is also an important event in ocular tissue, leading to diabetic retinopathy, glaucoma and blindness (Adonis et al., Am. J. Ophthal., 1994, 118, 445-450; Hammes et al., Nature Med., 1996, 2,529-533; Friedlander, et al., Natl. Acad. Sci. U.S.A., 1996, 93, 9764-9769) and in joints, promoting rheumatoid arthritis (Peacock et al., J. Exp. Med., 1992, 175, 1135-1138).

αᵥβ₃ has been shown to play a pivotal role in the proliferation and migration of smooth muscle and vascular endothelial cells, a pathological process leading to restenosis after balloon angioplasty (Choi et al., J. Vasc. Surgery, 1994, 19, 125-134; Matsumo et al., Circulation, 1994, 90, 2203-2206). At least one type of virus (adenovirus) has been shown to utilize αᵥβ₃ for entering host cells (White et al., Current Biology, 1993, 596-599.

Various bone diseases involve bone resorption which is mediated by only one known class of cells, the osteoclasts. When activated for resorption, these motile cells initially bind to bone, a process well known to be mediated by αᵥβ₃ (Davies et al., J. Cell. Biol., 1989 109, 1817-1826; Helfrich et al., J Bone Mineral Res., 1992, 7, 335-343). It is also well known that blockade of αᵥβ₃ with antibodies or RGD containing peptides block osteoclast cell adhesion and bone resorption in vitro (Horton et al., Exp. Cell Res. 1991, 195, 368-375) and that echistatin, an RGD containing protein, inhibits bone resorption in vivo (Fisher et al., Endocrinology, 1993, 132, 1411-1413). More recently, an RGD peptidomimetic has likewise been shown to inhibit osteoclasts in vitro and, by i.v. administration in vivo prevents osteoporosis (Engleman et al., J. Clin. Invest., 1997, 99, 2284-2292).

αᵥβ₃ also plays an important role in autoimmune diseases such as psoriasis and rheumatoid arthritis. Peacock, et al., supra.

Numerous patents/applications have claimed various non-peptide αᵥ62 ₃ inhibitors for some or all of the above applications (e.g. EP92307157.5A, EP92307156.7A, WO9708145, WO09532710, WO96/00730, WO9637492, WO9626190, WO9606087, WO97/23451, WO9724119, WO9724122, WO9724124, WO96-US20744961220, EP796855, WO9733887, WO97/34865, WO97/35615, WO97/36859, WO97/35615, WO97/08145, U.S. Pat. No. 5,668,159, WO98/08840, WO98/14192).

WO9532710 teaches compounds for inhibiting bone resorption. Among the preferred compounds are compounds having a 4-alkyloxy substituted benzoic acid core coupled to an (α-phenylsulfonylamino-3-amino propanoic acid) terminus. None of the exemplary compounds teach a 2-hydroxy substitution of the benzoyl core. The lead compound of WO9532710 exhibited limited bioavailability in vivo. (VnR symposium Abstracts, 211th ACS National Meeting, New Orleans, La., Mar. 24-28 (1996).)

WO9708145 discloses certain meta-guanidine, urea, thiourea and azacyclic amino substituted benzoic acid derivitaves as integrin antagonists.

European patent application number EP0320032 broadly claims certain 2-aminoalkoxy-substituted pyridazine derivatives. The compounds disclosed do not comprise an acid functionality.

WO9513262 teaches certain 2-hydroxy-4-heteroarylmethoxy benzamide derivatives are endothelin inhibitors.

### DETAILED DESCRIPTION OF THE INVENTION

According to the present invention are provided novel compounds of Formula I: wherein

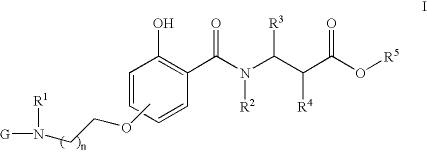

G is

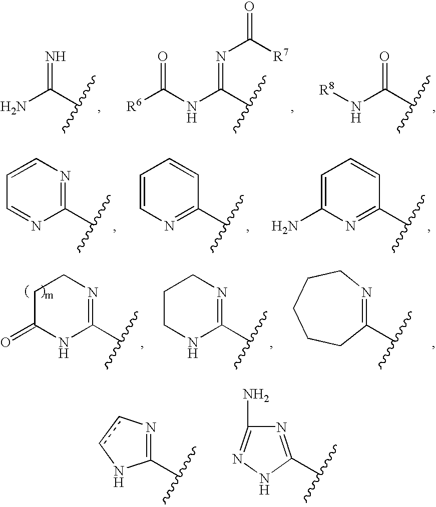

R¹ and R² are independently, hydrogen, alkyl of 1 to 6 carbon atoms, mono or bicyclic aralkyl of 6 to 10 carbon atoms, or heterocycloalkyl-alkyl comprised of a 5 to 10 membered mono or bicyclic heterocycloalkyl having 1 to 3 heteroatoms selected from S, N and O and an alkyl of 1 to 6 carbon atoms;

R³ is hydrogen, mono or bicyclic aryl of 6 to 10 carbon atoms, 5 to 10 membered mono or bicyclic heterocycloalkyl having 1 to 3 heteroatoms selected from S, N and O;

R⁴ is hydrogen, NHR⁹, OR⁹, NHCO₂R⁹, NHCONHR⁹, NHCOR⁹ or NHSO₂R⁹; provided that R³ and R⁴ are not both hydrogen;

R⁵ is hydrogen or alkyl of 1 to 6 carbon atoms which may optionally be substituted with a terminal group which serves as a prodrug. For example, the alkyl group may be substituted with an acid, alcohol or amino functionality to form an alkylamino, carboxyalkyl or alkanol group;

R⁶ and R⁷ are independently hydrogen, alkyl of 1 to 6 carbon atoms, alkoxy of 1 to 6 carbon atoms, or aralkoxy of 6 to 10 carbon atoms;

R⁸ and R⁹ are independently hydrogen, trichloroalkylalkoxy, trifluoromethoxyphenyl, aralkenyl of 7 to 10 carbon atoms, alkyl of 1 to 10 carbon atoms, alkenyl of 2 to 10 carbons, alkynyl of 2 to 10 carbons, mono or polycycloalkyl of 3-12 carbon atoms, mono or polycycloalkyl-alkyl of 4-12 carbon atoms, mono or bicyclic aryl of 6 to 10 carbon atoms, 6 to 10 membered mono or bicyclic heterocycloalkyl having 1 to 3 heteroatoms selected from S, N and O, mono or bicyclic aralkyl of 7 to 10 carbon atoms, or heterocycloalkyl-alkyl comprised of a 5 to 10 membered mono or bicyclic heterocycloalkyl having 1 to 3 heteroatoms selected from S, N and O and an alkyl of 1 to 6 carbon atoms;

n is an integer from 1 to 4; and m is 0 or 1; or a pharmaceutically acceptable salt thereof.

In some preferred embodiments of the present invention G is 6-aminopyridin-2yl, pyridin-2yl, pyrimidyl, tetrahydropyrimidyl, tetrahydropyrimid-4-one, dihydroimidazolyl, amino(imino)-, pyridyl-urea, benzyl-urea, or imidazolidinyl.

In a still more preferred embodiment of the present invention G is 6-aminopyridin-2-yl, pyridin-2yl, dihydroimidazolyl, 5-amino 1,2,4-triazol-4yl (and/or all tautomers thereof) or tetrahydropyrimidyl, R³ is H, and n is 2 or 3.

In some preferred aspects of the invention R9 is methyl, ethyl, n-propyl, i-propyl, allyl, homoallyl, propargyl, pentyl, n-hexyl, octyl, neopentyl, trichloroethyl, n-butyl, i-butyl, butynyl, phenyl, methylphenyl, dimethylphenyl, halophenyl, methoxyphenyl, acetylphenyl, biphenyl, naphthyl, benzyl, phenethyl, cyclohexyl, cyclohexylmethyl, trimethylcyclopropyl, phenylcyclopropyl, adamantyl, adamantylmethyl, cinnamic, pyridyl or dimethylfuranyl.

“Alkyl”, whether used alone or as part of a group such as “alkoxy”, means a branched or straight chain having from 1 to 10 carbon atoms. Exemplary alkyl groups include methyl, ethyl, propyl, isopropyl, butyl, isobutyl, t-butyl, pentyl and hexyl. Lower alkyl refers to alkyl having from 1 to 4 carbon atoms. Alkyl groups may be substituted.

“Cycloalkyl” as used herein refers to mono or polycyclic alkyl groups of 3-12 carbon atoms. Exemplary cycloalkyl groups include cyclopropyl, cyclohexyl and adamantyl. Cycloalkyl groups may be substituted. One preferred substitution is phenyl.

“Aryl” whether used alone or as part of a group such as “aralkyl”, means mono or bicyclic aromatic rings having from 6 to 10 carbon atoms. Exemplary aryl groups include phenyl and naphthyl. The aryl may be substituted with one or more substituents. Substituents for the alkyl, cycloalkyl and aryl groups herein include halogen, lower alkyl, alkoxy, alkythio, amino, nitro, cyano, carboxy, carboxyalkyl, alkanoyl, alkylamino, perhaloalkyl, hydroxy, oxy and phenyl. One preferred aryl substituent group is phenyl.

“Heterocycloalkyl” whether used alone or as part of a group such as “heterocycloalkyl-alkyl” means a stable, saturated or unsaturated 5 to 10 membered mono or bicyclic ring having from 1 to 3 heteroatoms selected from N, O and S. Exemplary heterocycloalkyls include pyrazinyl, pyrazolyl, tetrazolyl, furanyl, thienyl, pyridyl, imidazolyl, pyrimidinyl, tetrahydropyrimidinyl, isoxazolyl, thiazolyl, isothiazolyl, quinolinyl, indolyl, isoquinolinyl, oxazolyl and oxadiazolyl. Preferred heteroaryl groups include pyrimidinyl, tetrahydropyrimidinyl, pyridyl, and imidazolyl. Most preferred heteroaryls include pyridin-2yl, and tetrahydropyrimidine. The heteroaryl may also be substituted with one or more substituents. Substituents include halogen, lower alkyl, alkoxy, alkythio, amino, nitro, cyano, carboxy, carboxyalkyl, alkanoyl, alkylamino, perhaloalkyl, hydroxy, oxy and phenyl. Preferred substituents include amino and oxy. Preferred substituted heterocycloalkyls include 6 aminopyridin-2yl and tetrahydropyrimid-4-one.

“Aralkyl” means an aryl-alkyl group in which the aryl and alkyl are as previously described. Exemplary aralkyl groups include benzyl and phenethyl. Use in this context, the alkyl group may include one or more double bonds.

“Heterocycloalkyl-alkyl” means a heterocycloalkyl group in which the heterocycloalkyl and alkyl are as previously described. Use in this context, the alkyl group may include one or more double bonds. Exemplary heterocycloalkyl-alkyls include pyridylmethyl, pyridylethyl, thienylethyl, thienylmethyl, indolylmethyl, and furylmethyl.

“Alkoxy” means an alkyl-O group in which the alkyl group is as previously described. Exemplary alkoxy groups include methoxy, ethoxy, n-propoxy, i-propoxy, n-butoxy, and t-butoxy.

“Aralkoxy” means an aryl-alkoxy group in which aryl and alkoxy are as previously described.

“Halogen” includes fluorine, chlorine, iodine and bromine.

“Prodrug”, as used herein means a compound which is convertible in vivo by metabolic means (e.g. by hydrolysis) to a compound of Formula I.

NMR and IR spectra indicate the 2-hydroxy substitution of Formula I is strongly H-bonded to the adjacent carbonyl, effectively forming a six-membered ring which conformationally restricts the amide residue bearing the carboxy terminus. Thus, the 2-hydroxy substitution of the phenyl core of Formula I plays a significant role in integrin receptor selectivity.

In addition, the 2-hydroxy compounds of the invention are believed to obviate at least two of the three hydrating water molecules which are known to form intermolecular hydrogen bonds with secondary amide functionalities. The energy needed to desolvate water molecules for efficient transport across cell membranes is thus reduced in compounds of the present invention and is believed to contribute to the markedly improved plasma concentrations seen with compounds of the present invention.

Preferred compounds include:

(2S)-3-({2-hydroxy-4-[2-(1,4,5,6-tetrahydropyrimidin-2-ylamino)ethoxy]benzoyl}amino)-2-[(methoxycarbonyl)amino]propanoic acid,

(2S)-2-[(ethoxycarbonyl)amino]-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}amino)-2-[(propoxycarbonyl)amino]propanoic acid,

(2S)-3-({2-hydroxy-4-[2-(1,4,5,6-tetrahydropyrimidin-2-ylamino)ethoxy]benzoyl}amino)-2-[(isopropoxycarbonyl)amino]propanoic acid,

(2S)-2-{[(allyloxy)carbonyl]amino}-3-({2-hydroxy-4-[2-(1,4,5,6-tetrahydropyrimidin-2-ylamino)ethoxy]benzoyl}amino)propanoic acid,

(2S)-2-{[(but-3-enyloxy)carbonyl]amino}-3-({2-hydroxy-4-[2-(1,4,5,6-tetrahydropyrimidin-2-ylamino)ethoxy]benzoyl}amino)propanoic acid,

(2S)-2-{[(hexyloxy)carbonyl]amino}-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-{[(octyloxy)carbonyl]amino}propanoic acid,

(2S)-3-({2-hydroxy-4-[2-(1,4,5,6-tetrahydropyrimidin-2-ylamino)ethoxy]benzoyl}-amino)-2-{[(neopentyloxy)carbonyl]amino}propanoic acid,

(2S)-3-({2-hydroxy-4-[2-(1,4,5,6-tetrahydropyrimidin-2-ylamino)ethoxy]benzoyl}-amino)-2-{[(2,2,2-trichloroethoxy)carbonyl]amino}propanoic acid,

(2S)-3-({2-hydroxy-4-[2-(1,4,5,6-tetrahydropyrimidin-2-ylamino)ethoxy]benzoyl}-amino)-2-[(butoxycarbonyl)amino]propanoic acid,

(2S)-3-({2-hydroxy-4-[2-(1,4,5,6-tetrahydropyrimidin-2-ylamino)ethoxy]benzoyl}-amino)-2-[(isobutoxycarbonyl)amino]propanoic acid,

(2S)-3-({2-hydroxy-4-[2-(1,4,5,6-tetrahydropyrimidin-2-ylamino)ethoxy]benzoyl}-amino)-2-[(prop-2-ynyloxy)carbonyl]amino}propanoic acid,

(2S)-2-{[(benzyloxy)carbonyl]amino}-3-({2-hydroxy-4-[2-(1,4,5,6-tetrahydropyrimidin-2-ylamino)ethoxy]benzoyl}amino)propanoic acid,

(2S)-2-{[(butylamino)carbonyl]amino}-3-({2-hydroxy-4-[2-(1,4,5,6-tetrahydropyrimidin-2-ylamino)ethoxy]benzoyl}amino)propanoic acid,

(2S)-2-{[(hexylamino)carbonyl]amino}-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-{[(octylamino)carbonyl]amino}propanoic acid,

(2S)-2-{[(allylamino)carbonyl]amino}-3-({2-hydroxy-4-[2-(1,4,5,6-tetrahydropyrimidin-2-ylamino)ethoxy]benzoyl}amino)propanoic acid,

(2S)-2-{[(1-adamantylamino)carbonyl]amino}-3-({2-hydroxy-4-[2-(1,4,5,6-tetrahydropyrimidin-2-ylamino)ethoxy]benzoyl}amino)propanoic acid,

(2S)-2-[(anilinocarbonyl)amino]-3-({2-hydroxy-4-[2-(1,4,5,6-tetrahydropyrimidin-2-ylamino)ethoxy]benzoyl}amino)propanoic acid,

(2S)-2-{[(cyclohexylamino)carbonyl]amino}-3-({2-hydroxy-4-[2-(1,4,5,6-tetrahydropyrimidin-2-ylamino)ethoxy]benzoyl}amino)propanoic acid,

(2S)-2-{[(benzylamino)carbonyl]amino}-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-[(4-toluidinocarbonyl)amino]propanoic acid,

(2S)-3-({2-hydroxy-4-[2-(1,4,5,6-tetrahydropyrimidin-2-ylamino)ethoxy]benzoyl}-amino)-2-[(2-toluidinocarbonyl)amino]propanoic acid,

(2S)-3-({2-hydroxy-4-[2-(1,4,5,6-tetrahydropyrimidin-2-ylamino)ethoxy]benzoyl}-amino)-2-{[(2-methoxyanilino)carbonyl]amino}propanoic acid,

(2S)-3-({2-hydroxy-4-[2-(1,4,5,6-tetrahydropyrimidin-2-ylamino)ethoxy]benzoyl}-amino)-2-{[(4-methoxyanilino)carbonyl]amino}propanoic acid,

(2S)-2-{[(2-chloroanilino)carbonyl]amino}-3-({2-hydroxy-4-[2-(1,4,5,6-tetrahydropyrimidin-2-ylamino)ethoxy]benzoyl}amino)propanoic acid,

(2S)-2-{[(2-bromoanilino)carbonyl]amino}-3-({2-hydroxy-4-[2-(1,4,5,6-tetrahydropyrimidin-2-ylamino)ethoxy]benzoyl}amino)propanoic acid,

(2S)-2-{[([1,1′-biphenyl]-2-ylamino)carbonyl]amino}-3-({2-hydroxy-4-[2-(1,4,5,6-tetrahydropyrimidin-2-ylamino)ethoxy]benzoyl}amino)propanoic acid,

(2S)-2-{[(4-chloroanilino)carbonyl]amino}-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-{[(1-naphthylamino)carbonyl]amino}propanoic acid,

(2S)-3-({2-hydroxy-4-[2-(1,4,5,6-tetrahydropyrimidin-2-ylamino)ethoxy]benzoyl}-amino)-2-({[(2-phenylethyl)amino]carbonyl amino)propanoic acid,

(2S)-3-({2-hydroxy-4-[2-(1,4,5,6-tetrahydropyrimidin-2-ylamino)ethoxy]benzoyl}-amino)-2-(isobutyrylamino)propanoic acid,

(2S)-2-(hexanoylamino)-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]-benzoyl}amino)-2-(pentanoylamino)propanoic acid,

(2S)-2-[(3,3-dimethylbutanoyl)amino]-3-({2-hydroxy-4-[2-(1,4,5,6-tetrahydropyrimidin-2-ylamino)ethoxy]benzoyl}amino)propanoic acid,

(2S)-2-[(cyclohexylcarbonyl)amino]-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-[(2-phenylacetyl)amino]propanoic acid,

(2S)-3-({2-hydroxy-4-[2-(1,4,5,6-tetrahydropyrimidin-2-ylamino)ethoxy]benzoyl}-amino)-2-[(3-phenylpropanoyl)amino]propanoic acid,

(2S)-2-[(2-cyclohexylacetyl)amino]-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-{[(E)-3-phenylprop-2-enoyl]amino}propanoic acid,

(2S)-2-[(2-chlorobenzoyl)amino]-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-[(2-methylbenzoyl)amino]propanoic acid,

(2S)-3-({2-hydroxy-4-[2-(1,4,5,6-tetrahydropyrimidin-2-ylamino)ethoxy]benzoyl}-amino)-2-[(2-methoxybenzoyl)amino]propanoic acid,

(2S)-2-[(4-chlorobenzoyl)amino]-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(1,4,5,6-tetrahydropyrimidin-2-ylamino)ethoxy]benzoyl}-amino)-2-[(4-methylbenzoyl)amino]propanoic acid,

(2S)-3-({2-hydroxy-4-[2-(1,4,5,6-tetrahydropyrimidin-2-ylamino)ethoxy]benzoyl}-amino)-2-[(4-methoxybenzoyl)amino]propanoic acid,

(2S)-2-[(2,5-dimethyl-3-furoyl)amino]-3-({2-hydroxy-4-[2-(1,4,5,6-tetrahydropyrimidin-2-ylamino)ethoxy]benzoyl}amino)propanoic acid,

(2S)-2-[(2-bromobenzoyl)amino]-3-({2-hydroxy-4-[2-(1,4,5,6-tetrahydropyrimidin-2-ylamino)ethoxy]benzoyl}amino)propanoic acid,

(2S)-2-[(4-bromobenzoyl)amino]-3-({2-hydroxy-4-[2-(1,4,5,6-tetrahydropyrimidin-2-ylamino)ethoxy]benzoyl}amino)propanoic acid

(2S)-2-[(2,3-dimethylbenzoyl)amino]-3-({2-hydroxy-4-[2-(1,4,5,6-tetrahydropyrimidin-2-ylamino)ethoxy]benzoyl}amino)propanoic acid,

(2S)-2-[(3-chlorobenzoyl)amino]-3-({2-hydroxy-4-[2-(1,4,5,6-tetrahydropyrimidin-2-ylamino)ethoxy]benzoyl}amino)propanoic acid,

(2S)-3-({2-hydroxy-4-[2-(pyrimidin-2-ylamino)ethoxy]benzoyl}amino)-2-[(phenoxycarbonyl)amino]propanoic acid,

(2S)-2-{[(benzyloxy)carbonyl]amino}-3-({2-hydroxy-4-[2-(pyrimidin-2-ylamino)-ethoxy]benzoyl}amino)propanoic acid,

(2S)-3-({2-hydroxy-4-[2-(pyrimidin-2-ylamino)ethoxy]benzoyl}amino)-2-[(isobutoxycarbonyl)amino]propanoic acid,

(2S)-3-({2-hydroxy-4-[2-(pyrimidin-2-ylamino)ethoxy]benzoyl}amino)-2-{[(4-methoxyphenoxy)carbonyl]amino}propanoic acid,

(2S)-3-({2-hydroxy-4-[2-(pyrimidin-2-ylamino)ethoxy]benzoyl}amino)-2-{[(octyloxy)carbonyl]amino}propanoic acid,

(2S)-2-[(butoxycarbonyl)amino]-3-({2-hydroxy-4-[2-(pyrimidin-2-ylamino)ethoxy]-benzoyl}amino)propanoic acid,

(2S)-3-({2-hydroxy-4-[2-(pyrimidin-2-ylamino)ethoxy]benzoyl}amino)-2-{[(2,2,2-trichloroethoxy)carbonyl]amino}propanoic acid,

(2S)-3-({2-hydroxy-4-[2-(pyrimidin-2-ylamino)ethoxy]benzoyl}amino)-2-{[(neopentyloxy)carbonyl]amino}propanoic acid,

(2S)-3-({2-hydroxy-4-[2-(pyrimidin-2-ylamino)ethoxy]benzoyl}amino)-2-({[(4-nitrobenzyl)oxy]carbonyl}amino)propanoic acid,

(2S)-2-{[(hexyloxy)carbonyl]amino}-3-({2-hydroxy-4-[2-(pyrimidin-2-ylamino)ethoxy]benzoyl}amino)propanoic acid,

(2S)-3-({2-hydroxy-4-[2-(pyrimidin-2-ylamino)ethoxy]benzoyl}amino)-2-{[(prop-2-ynyloxy)carbonyl]amino}propanoic acid,

(2S)-3-({2-hydroxy-4-[2-(pyrimidin-2-ylamino)ethoxy]benzoyl}amino)-2-{[(4-methylphenoxy)carbonyl]amino}propanoic acid,

(2S)-3-({2-hydroxy-4-[2-(pyrimidin-2-ylamino)ethoxy]benzoyl}amino)-2-[(methoxycarbonyl)amino]propanoic acid,

(2S)-2-[(ethoxycarbonyl)amino]-3-({2-hydroxy-4-[2-(pyrimidin-2-ylamino)-ethoxy]benzoyl}amino)propanoic acid,

(2S)-3-({2-hydroxy-4-[2-(pyrimidin-2-ylamino)ethoxy]benzoyl}amino)-2-[(propoxycarbonyl)amino]propanoic acid,

(2S)-3-({2-hydroxy-4-[2-(pyrimidin-2-ylamino)ethoxy]benzoyl}amino)-2-[(isopropoxycarbonyl)amino]propanoic acid,

(2S)-2-{[(allyloxy)carbonyl]amino}-3-({2-hydroxy-4-[2-(pyrimidin-2-ylamino)-ethoxy]benzoyl}amino)propanoic acid,

(2S)-2-{[(but-3-enyloxy)carbonyl]amino}-3-({2-hydroxy-4-[2-(pyrimidin-2-ylamino)ethoxy]benzoyl}amino)propanoic acid,

(2S)-2-[(anilinocarbonyl)amino]-3-({2-hydroxy-4-[2-(pyrimidin-2-ylamino)-ethoxy]benzoyl}amino)propanoic acid,

(2S)-2-{[(tert-butylamino)carbonyl]amino}-3-({2-hydroxy-4-[2-(pyrimidin-2-ylamino)ethoxy]benzoyl}amino)propanoic acid,

(2S)-2-{[(butylamino)carbonyl]amino}-3-({2-hydroxy-4-[2-(pyrimidin-2-ylamino)ethoxy]benzoyl}amino)propanoic acid,

(2S)-3-({2-hydroxy-4-[2-(pyridin-2-ylamino)ethoxy]benzoyl}amino)-2-{[(4-methoxyanilino)carbonyl]amino}propanoic acid,

(2S)-2-{[(2-ethylanilino)carbonyl]amino}-3-({2-hydroxy-4-[2-(pyrimidin-2-ylamino)ethoxy]benzoyl}amino)propanoic acid,

(2S)-2-{[(allylamino)carbonyl]amino}-3-({2-hydroxy-4-[2-(pyrimidin-2-ylamino)ethoxy]benzoyl}amino)propanoic acid,

(2S)-2-{[(2,4-dichloroanilino)carbonyl]amino}-3-({2-hydroxy-4-[2-(pyrimidin-2-ylamino)ethoxy]benzoyl}amino)propanoic acid,

(2S)-3-({2-hydroxy-4-[2-(pyrimidin-2-ylamino)ethoxy]benzoyl}amino)-2-[(2-toluidinocarbonyl)amino]propanoic acid,

(2S)-3-({2-hydroxy-4-[2-(pyrimidin-2-ylamino)ethoxy]benzoyl}amino)-2-{[(2-methoxyanilino)carbonyl]amino}propanoic acid,

(2S)-2-{[(2-chloroanilino)carbonyl]amino}-3-({2-hydroxy-4-[2-pyrimidin-2-(ylamino)ethoxy]benzoyl}amino)propanoic acid,

(2S)-2-{[(2-bromoanilino)carbonyl]amino}-3-({2-hydroxy-4-[2-(pyrimidin-2-ylamino)ethoxy]benzoyl}amino)propanoic acid,

(2S)-2-{[([1,1′-biphenyl]-2-ylamino)carbonyl]amino}-3-({2-hydroxy-4-[2-pyrimidin-2-ylamino)ethoxy]benzoyl}amino)propanoic acid,

(2S)-3-({2-hydroxy-4-[2-(pyrimidin-2-ylamino)ethoxy]benzoyl}amino)-2-[(4-toluidinocarbonyl)amino]propanoic acid,

(2S)-3-({2-hydroxy-4-[2-(pyrimidin-2-ylamino)ethoxy]benzoyl}amino)-2-({[4-(trifluoromethyl)anilino]carbonyl}amino)propanoic acid,

(2S)-3-({2-hydroxy-4-[2-(pyrimidin-2-ylamino)ethoxy]benzoyl}amino)-2-({[4-(trifluoromethoxy)anilino]carbonyl}amino)propanoic acid,

(2S)-2-{[(4-chloroanilino)carbonyl]amino}-3-({2-hydroxy-4-[2-(pyrimidin-2-ylamino)ethoxy]benzoyl}amino)propanoic acid,

(2S)-2-{[(4-fluoroanilino)carbonyl]amino}-3-({2-hydroxy-4-[2-(pyrimidin-2-ylamino)ethoxy]benzoyl}amino)propanoic acid,

(2S)-2-{[(4-acetylanilino)carbonyl]amino}-3-({2-hydroxy-4-[2-(pyrimidin-2-ylamino)ethoxy]benzoyl}amino)propanoic acid,

(2S)-2-({[4-(ethoxycarbonyl)anilino]carbonyl}amino)-3-({2-hydroxy-4-[2-(pyrimidin-2-ylamino)ethoxy]benzoyl}amino)propanoic acid,

(2S)-2-{[(cyclohexylamino)carbonyl]amino}-3-({2-hydroxy-4-[2-(pyrimidin-2-ylamino)ethoxy]benzoyl}amino)propanoic acid,

(2S)-3-({2-hydroxy-4-[2-(pyrimidin-2-ylamino)ethoxy]benzoyl}amino)-2-{[(1-naphthylamino)carbonyl]amino}propanoic acid,

(2S)-2-{[(benzylamino)carbonyl]amino}-3-({2-hydroxy-4-[2-(pyrimidin-2-ylamino)ethoxy]benzoyl}amino)propanoic acid,

(2S)-2-({2-hydroxy-4-[2-(pyrimidin-2-ylamino)ethoxy]benzoyl}amino-2-({[(2-phenylethyl)amino]carbonyl}amino)propanoic acid,

(2S)-3-({2-hydroxy-4-[2-(pyrimidin-2-ylamino)ethoxy]benzoyl}amino)-2-{[(octylamino)carbonyl]amino}propanoic acid,

(2S)-2-{[(benzyloxy)carbonyl]amino}-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}amino)-2-[(methoxycarbonyl)amino]propanoic acid,

(2S)-3-({4-[2-(4,5-dihydro-1H-imidazol-2-ylamino)ethoxy]-2-hydroxybenzoyl}-amino)-2-[(ethoxycarbonyl)amino]propanoic acid,

(2S)-3-({4-[2-(4,5-dihydro-1H-imidazol-2-ylamino)ethoxy]-2-hydroxybenzoyl}-amino)-2-[(propoxycarbonyl)amino]propanoic acid,

(2S)-3-({4-[2-(4,5-dihydro-1H-imidazol-2-ylamino)ethoxy]-2-hydroxybenzoyl}-amino)-2-[(isopropoxycarbonyl)amino]propanoic acid,

(2S)-2-{[(allyloxy)carbonyl]amino}-3-({4-[2-(4,5-dihydro-1H-imidazol-2-ylamino)ethoxy]-2-hydroxybenzoyl}amino)propanoic acid,

(2S)-2-{[(but-3-enyloxy)carbonyl]amino}-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}amino)-2-{[(prop-2-ynyloxy)carbonyl]amino}propanoic acid,

(2S)-3-({4-[2-(4,5-dihydro-1H-imidazol-2-ylamino)ethoxy]-2-hydroxybenzoyl}amino)-2-{[(hexyloxy)carbonyl]amino}propanoic acid,

(2S)-3-({4-[2-(4,5-dihydro-1H-imidazol-2-ylamino)ethoxy]-2-hydroxybenzoyl}amino)-2-{[(octyloxy)carbonyl]amino}propanoic acid,

(2S)-3-({4-[2-(4,5-dihydro-1H-imidazol-2-ylamino)ethoxy]-2-hydroxybenzoyl}amino)-2-{[(neopentyloxy)carbonyl]amino}propanoic acid,

(2S)-2-[(butoxycarbonyl)amino]-3-({4-[2-(4,5-dihydro-1H-imidazol-2-ylamino)-ethoxy]-2-hydroxybenzoyl}amino)propanoic acid,

(2S)-3-({4-[2-(4,5-dihydro-1H-imidazol-2-ylamino)ethoxy]-2-hydroxybenzoyl}amino)-2-[(isobutoxycarbonyl)amino]propanoic acid,

(2S)-2-{[(butylamino)carbonyl]amino}-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-{[(hexylamino)carbonyl]amino}propanoic acid,

(2S)-3-({4-[2-(4,5-dihydro-1H-imidazol-2-ylamino)ethoxy]-2-hydroxybenzoyl}-amino)-2-{[(octylamino)carbonyl]amino}propanoic acid,

(2S)-2-{[(allylamino)carbonyl]amino}-3-({4-[2-(4,5-dihydro-1H-imidazol-2-ylamino)ethoxy]-2-hydroxybenzoyl}amino)propanoic acid,

(2S)-2-{[(cyclohexylamino)carbonyl]amino}-3-({4-[2-(4,5-dihydro-1H-imidazol-2-ylamino)ethoxy]-2-hydroxybenzoyl}amino)propanoic acid,

(2S)-2-{[(benzylamino)carbonyl]amino}-3-({4-[2-(4,5-dihydro-1H-imidazol-2-ylamino)ethoxy]-2-hydroxybenzoyl}amino)propanoic acid,

3-({4-[2-(2,5-dihydro-1H-imidazol-4-ylamino)ethoxy]-2-hydroxybenzoyl)amino)-N-55 [(1S,2R)-2-phenylcyclopropyl]amino)carbonyl)alanine,

(2S)-3-({4-[2-(4,5-dihydro-1H-imidazol-2-ylamino)ethoxy]-2-hydroxybenzoyl}-amino-2-{[(2-methoxyanilino)carbonyl]amino}propanoic acid,

(2S)-2-{[([1,1′-biphenyl]-2-ylamino)carbonyl]amino}-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-({[(2-phenylethyl)amino]carbonyl}amino)propanoic acid,

(2S)-3-({4-[2-(4,5-dihydro-1H-imidazol-2-ylamino)ethoxy]-2-hydroxybenzoyl}-amino)-2-(isobutyrylamino)propanoic acid,

(2S)-2-(butyrylamino)-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-(hexanoylamino)propanoic acid,

(2S)-3-({4-[2-(4,5-dihydro-1H-imidazol-2-ylamino)ethoxy]-2-hydroxybenzoyl}-amino)-2-(pentanoylamino)propanoic acid,

(2S)-3-({4-[2-(4,5-dihydro-1H-imidazol-2-ylamino)ethoxy]-2-hydroxybenzoyl}-amino)-2-[(3,3-dimethylbutanoyl)amino]propanoic acid,

(2S)-3-({4-[2-(4,5-dihydro-1H-imidazol-2-ylamino)ethoxy]-2-hydroxybenzoyl}-amino)-2-{[(2,2,3,3-tetramethylcyclopropyl)-carbonyl]amino}propanoic acid,

(2S)-2-{[2-(1-adamantyl)acetyl]amino}-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-(pent-4-ynoylamino)propanoic acid,

(2S)-2-[(cyclohexylcarbonyl)amino]-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-[(2-phenylacetyl)amino]propanoic acid,

(2S)-3-({4-[2-(4,5-dihydro-1H-imidazol-2-ylamino)ethoxy]-2-hydroxybenzoyl}-amino)-2-[(3-phenylpropanoyl)amino]propanoic acid,

(2S)-2-[(2-cyclohexylacetyl)amino]-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-{[(E)-3-phenylprop-2-enoyl]amino}propanoic acid,

(2S)-2-[(2-chlorobenzoyl)amino]-3-({4-[2-(4,5-dihydro-1H-imidazol-2-ylamino)-ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-[(2-methylbenzoyl)amino]propanoic acid,

(2S)-3-({4-[2-(4,5-dihydro-1H-imidazol-2-ylamino)ethoxy]-2-hydroxybenzoyl}-amino)-2-[(2-methoxybenzoyl)amino]propanoic acid,

(2S)-2-[(4-chlorobenzoyl)amino]-3-({4-[2-(4,5-dihydro-1H-imidazol-2-ylamino)-ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-[(4-methylbenzoyl)amino]propanoic acid,

(2S)-3-({4-[2-(4,5-dihydro-1H-imidazol-2-ylamino)ethoxy]-2-hydroxybenzoyl}-amino)-2-[(4-methoxybenzoyl)amino]propanoic acid,

(2S)-3-({4-[2-(4,5-dihydro-1H-imidazol-2-ylamino)ethoxy]-2-hydroxybenzoyl}-amino)-2-[(2,5-dimethyl-3-furoyl)amino]propanoic acid,

(2S)-2-[(2-bromobenzoyl)amino]-3-({4-[2-(4,5-dihydro-1H-imidazol-2-ylamino)-ethoxy]-2-hydroxybenzoyl}amino)propanoic acid,

(2S)-2-[(4-bromobenzoyl)amino]-3-({4-[2-(4,5-dihydro-1H-imidazol-2-ylamino)ethoxy]-2-hydroxybenzoyl}amino)propanoic acid,

(2S)-3-({4-[2-(4,5-dihydro-1H-imidazol-2-ylamino)ethoxy]-2-hydroxybenzoyl}-amino)-2-[(2,3-dimethylbenzoyl)amino]propanoic acid,

(2S)-2-[(3-chlorobenzoyl)amino]-3-({4-[2-(4,5-dihydro-1H-imidazol-2-ylamino)ethoxy]-2-hydroxybenzoyl}amino)propanoic acid,

(2S)-2-{[(benzyloxy)carbonyl]amino}-3-({2-hydroxy-4-[2-(3,4,5,6-tetrahydro-2H-azepin-7-ylamino)ethoxy]benzoyl}amino)propanoic acid,

(2S)-3-({2-hydroxy-4-[2-(3,4,5,6-tetrahydro-2H-azepin-7-ylamino)ethoxy]benzoyl}-amino)-2-[(methoxycarbonyl)amino]propanoic acid,

(2S)-2-[(ethoxycarbonyl)amino]-3-({2-hydroxy-4-[2-(3,4,5,6-tetrahydro-2H-azepin-7-ylamino)ethoxy]benzoyl}amino)propanoic acid,

(2S)-3-({2-hydroxy-4-[2-(3,4,5,6-tetrahydro-2H-azepin-7-ylamino)ethoxy]benzoyl}-amino)-2-[(propoxycarbonyl)amino]propanoic acid,

(2S)-3-({2-hydroxy-4-[2-(3,4,5,6-tetrahydro-2H-azepin-7-ylamino)ethoxy]benzoyl}-amino)-2-[(isopropoxycarbonyl)amino]propanoic acid,

(2S)-2-{[(allyloxy)carbonyl]amino}-3-({2-hydroxy-4-[2-(3,4,5,6-tetrahydro-2H-azepin-7-ylamino)ethoxy]benzoyl}amino)propanoic acid,

(2S)-2-{[(but-3-enyloxy)carbonyl]amino}-3-({2-hydroxy-4-[2-(3,4,5,6-tetrahydro-2H-azepin-7-ylamino)ethoxy]benzoyl}amino)propanoic acid,

(2S)-3-({2-hydroxy-4-[2-(3,4,5,6-tetrahydro-2H-azepin-7-ylamino)ethoxy]benzoyl }-amino)-2-{[(prop-2-ynyloxy)carbonyl]amino}propanoic acid,

(2S)-2-{[(hexyloxy)carbonyl]amino}-3-({2-hydroxy-4-[2-(3,4,5,6-tetrahydro-2H-azepin-7-ylamino)ethoxy]benzoyl}amino)propanoic acid,

(2S)-3-({2-hydroxy-4-[2-(3,4,5,6-tetrahydro-2H-azepin-7-ylamino)ethoxy]benzoyl}-amino)-2-{[(octyloxy)carbonyl]amino}propanoic acid,

(2S)-3-({2-hydroxy-4-[2-(3,4,5,6-tetrahydro-2H-azepin-7-ylamino)ethoxy]benzoyl}-amino)-2-{[(neopentyloxy)carbonyl]amino}propanoic acid,

(2S)-3-({2-hydroxy-4-[2-(3,4,5,6-tetrahydro-2H-azepin-7-ylamino)ethoxy]benzoyl}-amino)-2-{[(2,2,2-trichloroethoxy)carbonyl]amino}propanoic acid,

(2S)-2-[(butoxycarbonyl)amino]L-3-({2-hydroxy-4-[2-(3,4,5,6-tetrahydro-2H-azepin-7-ylamino)ethoxy]benzoyl}amino)propanoic acid,

(2S)-3-({2-hydroxy-4-[2-(3,4,5,6-tetrahydro-2H-azepin-7-ylamino)ethoxy]benzoyl}-amino)-2-[(isobutoxycarbonyl)amino]propanoic acid,

(2S)-3-{[4-(2-{[amino(imino)methyl]amino}ethoxy)-2-hydroxybenzoyl]amino}-2-{[(benzyloxy)carbonyl]amino}propanoic acid,

(2S)-3-{[4-(2-{[amino(imino)methyl]amino}ethoxy)-2-hydroxybenzoyl]amino}-2-[(methoxycarbonyl)amino]propanoic acid,

(2S)-3-{[4-(2-{[amino(imino)methyl]amino}ethoxy)-2-hydroxybenzoyl]amino}-2-[(ethoxycarbonyl)amino]propanoic acid,

(2S)-3-{[4-(2-{[amino(imino)methyl]amino}ethoxy)-2-hydroxybenzoyl]amino}-2-[(propoxycarbonyl)amino]propanoic acid,

(2S)-3-{[4-(2-{[amino(imino)methyl]amino}ethoxy)-2-hydroxybenzoyl]amino}-2-[(isopropoxycarbonyl)amino]propanoic acid,

(2S)-2-{[(allyloxy)carbonyl]amino}-3-{[4-(2-{[amino(imino)methyl]amino}ethoxy)-2-hydroxybenzoyl]amino}propanoic acid,

(2S)-3-{[4-(2-{[amino(imino)methyl]amino}ethoxy)-2-hydroxybenzoyl]amino}-2-{[(but-3-enyloxy)carbonyl]amino}propanoic acid,

(2S)-3-{[4-(2-{[amino(imino)methyl]amino}ethoxy)-2-hydroxybenzoyl]amino}-2-[(butoxycarbonyl)amino]propanoic acid,

(2S)-3-{[4-(2-{[amino(imino)methyl]amino}ethoxy)-2-hydroxybenzoyl]amino}-2-{[(2,2,2-trichloroethoxy)carbonyl]amino}propanoic acid,

(2S)-3-{[4-(2-{[amino(imino)methyl]amino}ethoxy)-2-hydroxybenzoyl]amino}-2-{[(neopentyloxy)carbonyl]amino}propanoic acid,

(2S)-3-{[4-(2-{[amino(imino)methyl]amino}ethoxy)-2-hydroxybenzoyl]amino}-2-{[(hexyloxy)carbonyl]amino}propanoic acid,

(2S)-3-{[4-(2-{[amino(imino)methyl]amino}ethoxy)-2-hydroxybenzoyl]amino}-2-{[(prop-2-ynyloxy)carbonyl]amino}propanoic acid,

(2S)-3-{[4-(2-{[amino(imino)methyl]amino}ethoxy)-2-hydroxybenzoyl]amino}-2-{[([1,1′-biphenyl]-2-ylmethoxy)carbonyl]amino}propanoic acid,

(2S)-3-{[4-(2-{[amino(imino)methyl]amino}ethoxy)-2-hydroxybenzoyl]amino}-2-({[(4-bromobenzyl)oxy]carbonyl}amino)propanoic acid,

(2S)-3-{[4-(2-{[amino(imino)methyl]amino}ethoxy)-2-hydroxybenzoyl]amino}-2-({[(4-fluorobenzyl)oxy]carbonyl}amino)propanoic acid,

(2S)-3-{[4-(2-{[amino(imino)methyl]amino}ethoxy)-2-hydroxybenzoyl]amino}-2-({[(2-bromobenzyl)oxy]carbonyl}amino)propanoic acid,

(2S)-3-{[4-(2-{[amino(imino)methyl]amino}ethoxy)-2-hydroxybenzoyl]amino}-2-[({[4-(trifluoromethyl)benzyl]oxy}carbonyl)amino]propanoic acid,

(2S)-3-{[4-(2-{[amino(imino)methyl]amino}ethoxy)-2-hydroxybenzoyl]amino}-2-[(2-toluidinocarbonyl)amino]propanoic acid,

(2S)-3-{[4-(2-{[amino(imino)methyl]amino}ethoxy)-2-hydroxybenzoyl]amino}-2-{[(2-methoxyanilino)carbonyl]amino}propanoic acid,

(2S)-3-{[4-(2-{[amino(imino)methyl]amino}ethoxy)-2-hydroxybenzoyl]amino}-2-{[(2-chloroanilino)carbonyl]amino}propanoic acid,

(2S)-3-{[4-(2-{[amino(imino)methyl]amino}ethoxy)-2-hydroxybenzoyl]amino}-2-}[(2-bromoanilino)carbonyl]amino}propanoic acid,

(2S)-3-{[4-(2-{[amino(imino)methyl]amino}ethoxy)-2-hydroxybenzoyl]amino}-2-{[([1,1′-biphenyl]-2-ylamino)carbonyl]amino}propanoic acid,

(2S)-3-{[4-(2-{[amino(imino)methyl]amino}ethoxy)-2-hydroxybenzoyl]amino}-2-[(4-toluidinocarbonyl)amino]propanoic acid,

(2S)-3-{[4-(2-{[amino(imino)methyl]amino}ethoxy)-2-hydroxybenzoyl]amino}-2-({[4-(trifluoromethoxy)anilino]carbonyl}amino)propanoic acid,

(2S)-3-{[4-(2-{[amino(imino)methyl]amino}ethoxy)-2-hydroxybenzoyl]amino}-2-{[(4-chloroanilino)carbonyl]amino}propanoic acid,

(2S)-3-{[4-(2-{[amino(imino)methyl]amino}ethoxy)-2-hydroxybenzoyl]amino}-2-{[(4-fluoroanilino)carbonyl]amino}propanoic acid,

(2S)-2-{[(4-acetylanilino)carbonyl]amino}-3-{[4-(2-{[amino(imino)methyl]amino}-ethoxy)-2-hydroxybenzoyl]amino}propanoic acid,

(2S)-3-{[4-(2-{[amino(imino)methyl]amino}ethoxy)-2-hydroxybenzoyl]amino}-2-{[(cyclohexylamino)carbonyl]amino}propanoic acid

(2S)-3-{[4-(2-{[amino(imino)methyl]amino}ethoxy)-2-hydroxybenzoyl]amino}-2-{[(1-naphthylamino)carbonyl]amino}propanoic acid,

(2S)-3-{[4-(2-{[amino(imino)methyl]amino}ethoxy)-2-hydroxybenzoyl]amino}-2-{[(benzylamino)carbonyl]amino}propanoic acid,

(2S)-3-{[4-(2-{[amino(imino)methyl]amino}ethoxy)-2-hydroxybenzoyl]amino}-2-({[(2-phenylethyl)amino]carbonyl}amino)propanoic acid,

(2S)-3-{[4-(2-{[amino(imino)methyl]amino}ethoxy)-2-hydroxybenzoyl]amino}-2-{[(octylamino)carbonyl]amino}propanoic acid,

(2S)-3-{[4-(2-{[amino(imino)methyl]amino}ethoxy)-2-hydroxybenzoyl]amino}-2-{[(4-methoxyanilino)carbonyl]amino}propanoic acid,

(2S)-3-{[4-(2-{[amino(imino)methyl]amino}ethoxy)-2-hydroxybenzoyl]amino}-2-[(anilinocarbonyl)amino]propanoic acid,

(2S)-3-{[4-(2-{[amino(imino)methyl]amino}ethoxy)-2-hydroxybenzoyl]amino}-2-(isobutyrylamino)propanoic acid,

(2S)-3-{[4-(2-{[amino(imino)methyl]amino}ethoxy)-2-hydroxybenzoyl]amino}-2-(butyrylamino)propanoic acid,

2S)-3-{[4-(2-{[amino(imino)methyl]amino}ethoxy)-2-hydroxybenzoyl]amino}-2-(hexanoylamino)propanoic acid,

(2S)-3-{[4-(2-{[amino(imino)methyl]amino}ethoxy)-2-hydroxybenzoyl]amino}-2-(pentanoylamino)propanoic acid,

(2S)-3-{[4-(2-{[amino(imino)methyl]amino}ethoxy)-2-hydroxybenzoyl]amino}-2-[(3,3-dimethylbutanoyl)amino]propanoic acid,

(2S)-3-{[4-(2-{[amino(imino)methyl]amino}ethoxy)-2-hydroxybenzoyl]amino}-2-{[(2,2,3,3-tetramethylcyclopropyl)carbonyl]amino}propanoic acid,

(2S)-2-{[2-(1-adamantyl)acetyl]amino}-3-{[4-(2-{[amino(imino)methyl]-amino}ethoxy)-2-hydroxybenzoyl]amino}propanoic acid,

(2S)-3-{[4-(2-{[amino(imino)methyl]amino}ethoxy)-2-hydroxybenzoyl]amino}-2-(pent-4-ynoylamino)propanoic acid,

(2S)-3-{[4-(2-{[amino(imino)methyl]amino}ethoxy)-2-hydroxybenzoyl]amino}-2-[(cyclohexylcarbonyl)amino]propanoic acid,

(2S)-3-{[4-(2-{[amino(imino)methyl]amino}ethoxy)-2-hydroxybenzoyl]amino}-2[(2-phenylacetyl)amino]propanoic acid,

(2S)-3-{[4-(2-{[amino(imino)methyl]amino}ethoxy)-2-hydroxybenzoyl]amino}-2[(2-phenylpropanoyl)amino]propanoic acid,

(2S)-3-{[4-(2-{[amino(imino)methyl]amino}ethoxy)-2-hydroxybenzoyl]amino}-2[(2-cyclohexylacetyl)amino]propanoic acid,

(2S)-3-{[4-(2-{[amino(imino)methyl]amino}ethoxy)-2-hydroxybenzoyl]amino}-2-{[(E)-3-phenylprop-2-enoyl]amino}propanoic acid,

(2S)-3-{[4-(2-{[amino(imino)methyl]amino}ethoxy)-2-hydroxybenzoyl]amino}-2-[(2-chlorobenzoyl)amino]propanoic acid,

(2S)-3-{[4-(2-{[amino(imino)methyl]amino}ethoxy)-2-hydroxybenzoyl]amino}-2[(2-methylbenzoyl)amino]propanoic acid,

(2S)-3-{[4-(2-{[amino(imino)methyl]amino}ethoxy)-2-hydroxybenzoyl]amino}-2-[(2-methoxybenzoyl)amino]propanoic acid,

(2S)-3-{[4-(2-{[amino(imino)methyl]amino}ethoxy)-2-hydroxybenzoyl]amino}-2[(4-chlorobenzoyl)amino]propanoic acid,

(2S)-3-{[4-(2-{[amino(imino)methyl]amino}ethoxy)-2-hydroxybenzoyl]amino}-2[(4-methylbenzoyl)amino]propanoic acid,

(2S)-3-{[4-(2-{[amino(imino)methyl]amino}ethoxy)-2-hydroxybenzoyl]amino}-2[(4-methoxybenzoyl)amino]propanoic acid,

(2S)-3-{[4-(2-{[amino(imino)methyl]amino}ethoxy)-2-hydroxybenzoyl]amino}-2-[(pyridin-3-ylcarbonyl)amino]propanoic acid,

(2S)-3-{[4-(2-{[amino(imino)methyl]amino}ethoxy)-2-hydroxybenzoyl]amino}-2-(isonicotinoylamino)propanoic acid,

(2S)-3-{[4-(2-{[amino(imino)methyl]amino}ethoxy)-2-hydroxybenzoyl]amino}-2-[(2,5-dimethyl-3-furoyl)amino]propanoic acid,

(2S)-3-{[4-(2-{[amino(imino)methyl]amino}ethoxy)-2-hydroxybenzoyl]amino}-2-[(2-bromobenzoyl)amino]propanoic acid,

(2S)-3-{[4-(2-{[amino(imino)methyl]amino}ethoxy)-2-hydroxybenzoyl]amino}-2[(4-bromobenzoyl)amino]propanoic acid,

(2S)-3-{[4-(2-{[amino(imino)methyl]amino}ethoxy)-2-hydroxybenzoyl]amino}-2-[(2,3-dimethylbenzoyl)amino]propanoic acid,

(2S)-3-{[4-(2-{[amino(imino)methyl]amino}ethoxy)-2-hydroxybenzoyl]amino}-2-[(3-chlorobenzoyl)amino]propanoic acid,

(2S)-3-{[4-(2-{[amino(imino)methyl]amino}ethoxy)-2-hydroxybenzoyl]amino}-2-(benzoylamino)propanoic acid,

(2S)-3-{[4-(2-{[amino(imino)methyl]amino}ethoxy)-2-hydroxybenzoyl]amino}-2-[(4-ethylbenzoyl)amino]propanoic acid,

(2S)-3-{[4-(2-{[amino(imino)methyl]amino}ethoxy)-2-hydroxybenzoyl]amino}-2[(4-butoxybenzoyl)amino]propanoic acid,

(2S)-3-{[4-(2-{[(benzylamino)carbonyl]amino}ethoxy)-2-hydroxybenzoyl]amino}-2-}[(benzyloxy)carbonyl]amino}propanoic acid,

(2S)-3-{[4-(2-{[(benzylamino)carbonyl]amino}ethoxy)-2-hydroxybenzoyl]amino}-2-[(methoxycarbonyl)amino]propanoic acid,

(2S)-3-{[4-(2-{[(benzylamino)carbonyl]amino}ethoxy)-2-hydroxybenzoyl]amino}-2-[(ethoxycarbonyl)amino]propanoic acid,

(2S)-3-{[4-(2-{[(benzylamino)carbonyl]amino}ethoxy)-2-hydroxybenzoyl]amino}-2-[(propoxycarbonyl)amino]propanoic acid,

(2S)-3-{[4-(2-{[(benzylamino)carbonyl]amino}ethoxy)-2-hydroxybenzoyl]amino}-2-[(isopropoxycarbonyl)amino]propanoic acid,

(2S)-2-{[(allyloxy)carbonyl]amino}-3-{[4-(2-{[(benzylamino)carbonyl]amino}-ethoxy)-2-hydroxybenzoyl]amino}propanoic acid,

(2S)-3-{[4-(2-{[(benzylamino)carbonyl]amino}ethoxy)-2-hydroxybenzoyl]amino}-2-{[(but-3-enyloxy)carbonyl]amino}propanoic acid,

(2S)-3-{[4-(2-{[(benzylamino)carbonyl]amino}ethoxy)-2-hydroxybenzoyl]amino}-2-{[(prop-2-ynyloxy)carbonyl]amino}propanoic acid,

(2S)-3-{[4-(2-{[(benzylamino)carbonyl]amino}ethoxy)-2-hydroxybenzoyl]amino}-2-}[(hexyloxy)carbonyl]amino}propanoic acid,

(2S)-3-{[4-(2-{[(benzylamino)carbonyl]amino}ethoxy)-2-hydroxybenzoyl]amino}-2-{[(octyloxy)carbonyl]amino}propanoic acid,

(2S)-3-{[4-(2-{[(benzylamino)carbonyl]amino}ethoxy)-2-hydroxybenzoyl]amino}-2-{[(neopentyloxy)carbonyl]amino}propanoic acid,

(2S)-3-{[4-(2-{[(benzylamino)carbonyl]amino}ethoxy)-2-hydroxybenzoyl]amino}-2-{[(2,2,2-trichloroethoxy)carbonyl]amino}propanoic acid,

(2S)-3-{[4-(2-{[(benzylamino)carbonyl]amino}ethoxy)-2-hydroxybenzoyl]amino}-2-[(butoxycarbonyl)amino]propanoic acid,

(2S)-3-{[4-(2-{[(benzylamino)carbonyl]amino}ethoxy)-2-hydroxybenzoyl]amino}-2-[(isobutoxycarbonyl)amino]propanoic acid,

(2S)-2-{[(benzyloxy)carbonyl]amino}-3-({2-hydroxy-4-[2-({[(pyridin-3-ylmethyl)amino]carbonyl}amino)ethoxy]benzoyl}amino)propanoic acid,

(2S)-3-({2-hydroxy-4-[2-({[(pyridin-3-ylmethyl)amino]carbonyl}amino)ethoxy]-benzoyl}amino)-2-[(methoxycarbonyl)amino]propanoic acid,

(2S)-2-[(ethoxycarbonyl)amino]-3-({2-hydroxy-4-[2-({[(pyridin-3-ylmethyl)amino]carbonyl}amino)ethoxy]benzoyl}amino)propanoic acid,

(2S)-3-({2-hydroxy-4-[2-({[(pyridin-3-ylmethyl)amino]carbonyl}amino)-ethoxy]benzoyl}amino)-2-[(propoxycarbonyl)amino]propanoic acid,

(2S)-3-({2-hydroxy-4-[2-({[(pyridin-3-ylmethyl)amino]carbonyl}amino)-ethoxy]benzoyl}amino)-2-[(isopropoxycarbonyl)amino]propanoic acid,

(2S)-2-{[(allyloxy)carbonyl]amino}-3-({2-hydroxy-4-[2-({[(pyridin-3-ylmethyl)amino]carbonyl}amino)ethoxy]benzoyl}amino)propanoic acid,

(2S)-2-}[(but-3-enyloxy)carbonyl]amino}-3-({2-hydroxy-4-[2-({[(pyridin-3-ylmethyl)amino]carbonyl}amino)ethoxy]benzoyl}amino)propanoic acid,

(2S)-3-({2-hydroxy-4-[2-({[(pyridin-3-ylmethyl)amino]carbonyl}amino)-ethoxy]benzoyl}amino)-2-{[(prop-2-ynyloxy)carbonyl]amino}propanoic acid,

(2S)-2-{[(hexyloxy)carbonyl]amino}-3-({2-hydroxy-4-[2-({[(pyridin-3-ylmethyl)amino]carbonyl}amino)ethoxy]benzoyl}amino)propanoic acid,

(2S)-3-({2-hydroxy-4-[2-({[(pyridin-3-ylmethyl)amino]carbonyl}amino)-ethoxy]benzoyl}amino)-2-{[(octyloxy)carbonyl]amino}propanoic acid,

(2S)-3-({2-hydroxy-4-[2-({[(pyridin-3-ylmethyl)amino]carbonyl}amino)-ethoxy]benzoyl}amino)-2-{[(neopentyloxy)carbonyl]amino}propanoic acid,

(2S)-3-({2-hydroxy-4-[2-({[(pyridin-3-ylmethyl)amino]carbonyl}amino)ethoxy]-benzoyl}amino)-2-{[(2,2,2-trichloroethoxy)carbonyl]amino}propanoic acid,

(2S)-2-[(butoxycarbonyl)amino]-3-({2-hydroxy-4-[2-({[(pyridin-3-ylmethyl)amino]-carbonyl}amino)ethoxy]benzoyl}amino)propanoic acid,

(2S)-3-({2-hydroxy-4-[2-({[(pyridin-3-ylmethyl)amino]carbonyl}amino)-ethoxy]benzoyl}amino)-2-[(isobutoxycarbonyl)amino]propanoic acid,

(2S)-2-{[(benzyloxy)carbonyl]amino}-3-({2-hydroxy-4-[2-({[(pyridin-4-ylmethyl)amino]carbonyl}amino)ethoxy]benzoyl}amino)propanoic acid,

(2S)-3-({2-hydroxy-4-[2-({[(pyridin-4-ylmethyl)amino]carbonyl}amino)-ethoxy]benzoyl}amino)-2-[(methoxycarbonyl)amino]propanoic acid,

(2S)-2-[(ethoxycarbonyl)amino]-3-({2-hydroxy-4-[2-({[(pyridin-4-ylmethyl)amino]carbonyl}amino)ethoxy]benzoyl}amino)propanoic acid,

(2S)-3-({2-hydroxy-4-[2-({[(pyridin-4-ylmethyl)amino]carbonyl}amino)-ethoxy]benzoyl}amino)-2-[(propoxycarbonyl)amino]propanoic acid,

(2S)-3-({2-hydroxy-4-[2-({[(pyridin-4-ylmethyl)amino]carbonyl}amino)-ethoxy]benzoyl}amino)-2-[(isopropoxycarbonyl)amino]propanoic acid,

(2S)-2-{[(allyloxy)carbonyl]amino}-3-({2-hydroxy-4-[2-({[(pyridin-4-ylmethyl)amino]carbonyl}amino)ethoxy]benzoyl}amino)propanoic acid,

(2S)-2-{[(but-3-enyloxy)carbonyl]amino}-3-({2-hydroxy-4-[2-({[(pyridin-4-ylmethyl)amino]carbonyl}amino)ethoxy]benzoyl}amino)propanoic acid,

(2S)-3-({2-hydroxy-4-[2-({[(pyridin-4-ylmethyl)amino]carbonyl}amino)-ethoxy]benzoyl}amino)-2-{[(prop-2-ynyloxy)carbonyl]amino}propanoic acid,

(2S)-2-{[(hexyloxy)carbonyl]amino}-3-({2-hydroxy-4-[2-({[(pyridin-4-ylmethyl)amino]carbonyl}amino)ethoxy]benzoyl}amino)propanoic acid,

(2S)-3-({2-hydroxy-4-[2-({[(pyridin-4-ylmethyl)amino]carbonyl}amino)-ethoxy]benzoyl}amino)-2-{[(octyloxy)carbonyl]amino}propanoic acid,

(2S)-3-({2-hydroxy-4-[2-({[(pyridin-4-ylmethyl)amino]carbonyl}amino)-ethoxy]benzoyl}amino)-2-{[(neopentyloxy)carbonyl]amino}propanoic acid,

(2S)-3-({2-hydroxy-4-[2-({[(pyridin-4-ylmethyl)amino]carbonyl}amino)-ethoxy]benzoyl}amino)-2-{[(2,2,2-trichloroethoxy)carbonyl]amino}propanoic acid,

(2S)-2-[(butoxycarbonyl)amino]-3-({2-hydroxy-4-[2-({[(pyridin-4-ylmethyl)amino]carbonyl}amino)ethoxy]benzoyl}amino)propanoic acid,

(2S)-3-({2-hydroxy-4-[2-({[(pyridin-4-ylmethyl)amino]carbonyl}amino)-ethoxy]benzoyl}amino)-2-[(isobutoxycarbonyl)amino]propanoic acid,

(2S)-2-{[(benzyloxy)carbonyl]amino}-3-({2-hydroxy-4-[2-({[(4-methylbenzyl)-amino]carbonyl}amino)ethoxy]benzoyl}amino)propanoic acid,

(2S)-2-{[(benzyloxy)carbonyl]amino}-3-({2-hydroxy-4-[2-({[(4-methylbenzyl)-amino]carbonyl}amino)ethoxy]benzoyl}amino)propanoic acid,

(2S)-2-{[(benzyloxy)carbonyl]amino}-3-({4-[2-({[(4-chlorobenzyl)amino]carbonyl}-amino)ethoxy]-2-hydroxybenzoyl}amino)propanoic acid,

(2S)-2-{[(benzyloxy)carbonyl]amino}-3-[(4-{2-[({[4-(dimethylamino)benzyl]amino}-carbonyl)amino]ethoxy -2-hydroxybenzoyl)amino]propanoic acid,

(2S)-3-[(4-{2-[({[4-(aminosulfonyl)benzyl]amino}carbonyl)amino]ethoxy}-2-hydroxybenzoyl)amino]-2-{[(benzyloxy)carbonyl]amino}propanoic acid,

(2S)-2-{[(benzyloxy)carbonyl]amino}-3-[(2-hydroxy-4-{2-[({[4-(trifluoromethoxy)-benzyl]amino}carbonyl)amino]ethoxy}benzoyl)amino]propanoic acid,

(2S)-2-{[(benzyloxy)carbonyl]amino}-3-({4-[2-(}[(2-chlorobenzyl)amino]carbonyl}-amino)ethoxy]-2-hydroxybenzoyl}amino)propanoic acid,

(2S)-2-{[(benzyloxy)carbonyl]amino}-3-({2-hydroxy-4-[2-({[(2-methylbenzyl)-amino]carbonyl}amino)ethoxy]benzoyl}amino)propanoic acid,

(2S)-2-{[(benzyloxy)carbonyl]amino}-3-({4-[2-({[(2-bromobenzyl)amino]-carbonyl}amino)ethoxy]-2-hydroxybenzoyl}amino)propanoic acid,

(2S)-2-{[(benzyloxy)carbonyl]amino}-3-({4-[2-({[(2,4-dichlorobenzyl)amino]-carbonyl}amino)ethoxy]-2-hydroxybenzoyl}amino)propanoic acid,

(2S)-3-({4-[2-({[(2-aminobenzyl)amino]carbonyl}amino)ethoxy]-2-hydroxybenzoyl}-amino)-2-{[(benzyloxy)carbonyl]amino}propanoic acid,

(2S)-2-{[(benzyloxy)carbonyl]amino}-3-({2-hydroxy-4-[2-({[(pyridin-2-ylmethyl)-amino]carbonyl}amino)ethoxy]benzoyl}amino)propanoic acid,

(2S)-2-Benzenesulfonylamino-3-(2-hydroxy-4-[3-(1,4,5,6-tetrahydropyrimidin-2-ylamino)-propoxy]-benzoylamino)-propionic acid,

(2S)-2-Benzenesulfonylamino-3-{2-hydroxy-4-[2-(1,4,5,6-tetrahydropyrimidin-2-ylamino)-ethoxy]-benzoylamino}-propionic acid tert-butyl ester,

(2S)-2-Benzenesulfonylamino-3-{2-hydroxy-5-[4-(pyrimidin-2-ylamino)-butoxy]-benzoylamino}-propionic acid,

3-{2-Hydroxy-5-[3-(1,4,5,6-tetrahydropyrimidin-2-ylamino)-propoxyl-benzoylamino)-3-phenyl-propionic acid ethyl ester,

(2S)-2-Benzenesulfonylamino-3-{2-hydroxy-4-[2-(1,4,5,6-tetrahydropyrimidin-2-ylamino)-ethoxy]-benzoylamino -propionic acid 2-(2-tert-butoxy-carbonylamino-ethoxy)-ethyl ester,

(2S)-2-Benzenesulfonylamino-3-{2-hydroxy-5-[4-(1,4,5,6-tetrahydropyrimidin-2-ylamino)-butoxy]-benzoylamino}-propionic acid ethyl ester,

(2S)-2-Benzenesulfonylamino-3-{2-hydroxy-4-[3-(pyrimidin-2-ylamino)-propoxy]-benzoylamino}-propionic acid,

3-2-Hydroxy-5-[3-(pyrimidin-2-ylamino)-propoxy]-benzoylamino}-3-phenyl-propionic acid,

(2S)-2-{Adamantan-1-yloxycarbonylamino)-3-{2-hydroxy-4-[2-(1,4,5,6-tetrahydropyrimidin-2-ylamino)-ethoxy]-benzoylamino)-propionic acid,

(2S)-2-Benzenesulfonylamino-3-(2-hydroxy-4-[3-(1,4,5,6-tetrahydropyrimidin-2-ylamino)-propoxy]-benzoylamino)-propionic acid ethyl ester,

3-{2-Hydroxy-5-[3-(pyrimidin-2-ylamino)-propoxy)-benzoylamino)-3-phenyl-propionic acid ethyl ester,

(2S)-2-(Adamantan-1-ylmethoxycarbonylamino)-3-{2-hydroxy-4-[2-(1,4,5,6-tetrahydropyrimidin-2-ylamino)-ethoxy]-benzoylamino}-propionic acid,

(2S)-2-Benzenesulfonylamino-3-{2-hydroxy-4-[2-(1,4,5,6-tetrahydropyrimidin-2-ylamino)-ethoxy]-benzoylamino}-propionic acid isopropyl ester,

(2S)-2-tert-Butoxycarbonylamino-3-{2-hydroxy-4-[2-(1,4,5,6-tetrahydropyrimidin-2-ylamino)-ethoxy]-benzoylamino}-propionic acid,

(2S)-2-Benzenesulfonylamino-3-{2-hydroxy-5-[4-(1,4,5,6-tetrahydropyrimidin-2-ylamino)-butoxy]-benzoylamino}-propionic acid,

2(S)-Benzenesulfonylamino-3-[2-hydroxy-4-(2-pyrimidin-2-ylamino)ethoxy]-benzoylamino]propionic acid ethyl ester,

2(S)-Benzenesulfonylamino-3-[2-hydroxy-4-(2-pyrimidin-2-ylamino)ethoxy]-benzoylamino]propionic acid,

2(S)-Benzenesulfonylamino-3-[2-hydroxy-4-[2-(3,4,5,6-tetrahydro-pyrimidin-2-ylamino)ethoxy]benzoylamino]propionic acid hydrochloride,

2(S)-Benzenesulfonylamino-3-[2-hydroxy-4-(2-pyrimidin-2-ylamino)ethoxy]-benzoylamino]propionic acid ethyl ester hydrochloride,

2(S)-Benzyloxycarbonylamino-3-[2-hydroxy-4-[2-(3,4,5,6-tetrahydropyrimidin-2-ylamino)ethoxy]benzoylamino]propionic acid ethyl ester hydrochloride,

2(S)-Benzyloxycarbonylamino-3-[2-hydroxy-4-[2-(3,4,5,6-tetrahydropyrimidin-2-ylamino)ethoxy]benzoylamino]propionic acid hydrochloride,

3-[4-(2-Guanidinoethoxy)-2-hydroxy-benzoylamino]-3-phenylpropanoic acid ethyl ester hydrochloride,

3-[4-(2-Guanidinoethoxy)-2-hydroxy-benzoylamino]-3-phenylpropanoic acid hydrochloride,

3-[2-hydroxy-4-[2-(pyrimidin-2-ylamino)-ethoxy]benzoylamino]-3-pyridin-3-yl-propanoic acid ethyl ester,

3-[2-hydroxy-4-[2-(pyrimidin-2-ylamino)-ethoxy]benzoylamino]-3-pyridin-3-yl-propanoic acid,

3-[2-hydroxy-4-[2-(3,4,5,6-tetrahydropyrimidin-2-ylamino)ethoxy]benzoyl-amino]-3-pyridin-3-yl-propanoic acid ethyl ester dihydro-chloride,

3-[2-hydroxy-4-[2-(3,4,5,6-tetrahydropyrimidin-2-ylamino)ethoxy]benzoyl-amino]-3-pyridin-3-yl-propanoic acid,

3-[4-(2-Guanidino-ethoxy)-2-hydroxybenzoyl-amino]-3-pyridin-3-yl-propanoic acid ethyl ester dihydrochloride,

3-[4-(2-Guanidino-ethoxy)-2-hydroxybenzoyl-amino]-3-pyridin-3-yl-propanoic acid,

3-[2-hydroxy-4-[2-(pyrimidin-2-ylamino)-ethoxy]benzoyl-amino]-3-phenyl-propanoic acid ethyl ester,

3-[2-hydroxy-4-[2-(pyrimidin-2-ylamino)-ethoxy]benzoyl-amino]-3-phenyl-propanoic acid hydrochloride,

3-[2-hydroxy-4-[2-(3,4,5,6-tetrahydropyrimidin-2-ylamino)ethoxy]-benzoylamino]-3-phenyl-propanoic acid ethyl ester hydrochloride,

3-[2-hydroxy-4-[2-(3,4,5,6-tetrahydropyrimidin-2-ylamino)ethoxy]-benzoylamino]-3-phenyl-propanoic acid,

3-[2-hydroxy-5-[3-(pyrimidin-2-ylamino)-propoxy]-benzoylamino]-3-phenyl-propanoic acid ethyl ester,

3-[2-hydroxy-5-[3-(pyrimidin-2-ylamino)-propoxy]-benzoylamino]-3-phenyl-propanoic acid,

3-[2-hydroxy-5-[3-(3,4,5,6-tetrahydropyrimidin-2-ylamino)propoxy]-benzoylamino]-3-phenyl-propanoic acid ethyl ester hydrochloride,

3-[2-hydroxy-5-[3-(3,4,5,6-tetrahydropyrimidin-2-ylamino)propoxy]-benzoylamino]-3-phenyl-propanoic,

2(S)-Benzyloxycarbonylamino-3-[2-hydroxy-4-[2-(pyrimidin-2-ylamino)ethoxy]-benzoylamino]propionic acid ethyl ester hydro-chloride,

2(S)-Benzyloxycarbonylamino-3-[2-hydroxy-4-[2-(pyrimidin-2-ylamino)ethoxy]-benzoylamino]propionic acid methyl ester,

2(S)-Benzyloxycarbonylamino-3-[2-hydroxy-4-[2-(pyrimidin-2-ylamino)ethoxy]-benzoylamino]propionic acid,

2(S)-Benzenesulfonylamino-3-[2-hydroxy-4-(2-methyl-pyrimidin-2-ylamino)-ethoxy]benzoylamino]propionic acid, and

2-Amino-3-[2-hydroxy-4-[2-(3,4,5,6-tetra-hydropyrimidin-2-ylamino)ethoxy]-benzoylamino]propionic acid dihydrochloride, or pharmaceutical salts thereof.

It is understood that the definition of compounds of Formula (I) include racemates, (racemic mixtures) and individual enantiomers or diastereomers. All asymmetric forms, individual isomers and combinations thereof are within the scope of the present invention.

Optically active isomers may be prepared, for example by resolving the racemic mixtures. The resolution can be carried out by methods known to those skilled in the art such as in the presence of resolving agent, by chiral chromatography, or combinations thereof.

Compounds of Formula I are useful in methods of selectively inhibiting or antagonizing an integrin receptor such as aᵥb₃. More specifically, methods of the present invention include but are not limited to methods of inhibiting cancer (tumor metastasis, tumorgenesis/tumor growth), angiogenesis (as in cancer, diabetic retinopathy, rheumatoid arthritis), restenosis (following balloon angioplasty or stent implantation), inflammation (as in rheumatoid arthritis, psoriasis), bone diseases (osteopenia induced by bone matastases, immobilization and glucocortocoid treatment, periodontal disease, hyperparathyroidism and rheumatoid arthritis), and viral infection by administration of a therapeutically effective amount of a compound of Formula I, or a pharmaceutically acceptable salt thereof.

The compounds of this invention are prepared in accordance with the solid phase combinatorial library synthesis methods or solution phase synthesis methods.

Generally, to prepare the compounds via combinatorial methodology, the starting acykesorcinol ester is condensed with an alkylene chain bearing a terminal primary amino group which is suitably blocked/protected. Methods for this condensation include, but are not limited to selective alkylation of one (the non-H-bonded hydroxyl group) of the resorcinol hydroxy groups, using standard procedures such as the Gabriel synthesis (Angew Chem. Int. Ed. Engl. 7, 1968, 919-930 (1968) or Mitsunobu reaction (Synthesis, 1981,1-28). After conventional deprotection of the N-terminus and the acid, the amine 2-2 was protected as fluorenylmethoxy carbamate (Fmoc) 2-6. On the other hand, in the case of G=pyrimidine 2-3, the primary amine 2-2 was activated with trimethylsilyl chloride in the presence of 2-bromopyrimidine in refluxing (anhydrous) 1,4-dioxane. The carboxylic acid 2-3 was activated as pentafluorophenyl ester 2-5. The carboxylic acid 2-3 was also hydrogenated under catalytic hydrogenation conditions to obtain the tetrahydropyrimidine derivative 2-4.

Orthogonally protected 2,3-diamino propionic acid 1-1 was used for carboxylic acid terminus and was immobilized on polymer support with linkers like but not limited to Wang. The 2-amino group of the 2,3-diaminopropionic acid was Fmoc protected, while the 3-amino group was dde (4,4-dimethyl-2,6-dioxocyclohex-1-ylideneethyl) protected. The 2-amino group was deprotected and further derivatized to 1-4, 1-5, 1-6 and 1-7 using various acylating agents including but not limitted to chloroformates, isocyanates, sulfonyl chlorides, carboxylic acids/chlorides. The 3-amino group was deprotected to give 1-8, 1-9, 1-10 and 1-11 and coupled with the resorcinol acid derivatives 2-4, 2-5 or 2-6.

N-terminus derivatives such as dihydroimidazole 5-3, azepine 5-4, guanidine 6-3, and ureas 6-4 were prepared from common primary amine intermediate 4-2.

Schemes 1-6, below, demonstrate the solid phase synthesis practice of this invention as it relates to the examples specified. Detailed synthetic procedures for representative compounds of this invention follow. Throughout the Examples data for LC (@254 nM) were obtained under the following conditions. HP 1100, 23oC, 10 μL injected; Column: YMC-ODS-A 4.6×50 5 g; Gradient A: 0.05% TFA/Water, B: 0.05% TFA/Acetonitrile Time 0 & 1 min: 98% A &2% B; 7 min: 10% A & 90% B; 8 min: 10% A & 90% B; 8.9 min: 98% A & 2%B; Post time 1 min; Flow rate 2.5 mL min.; Detection: 215 and 254 nm, DAD.

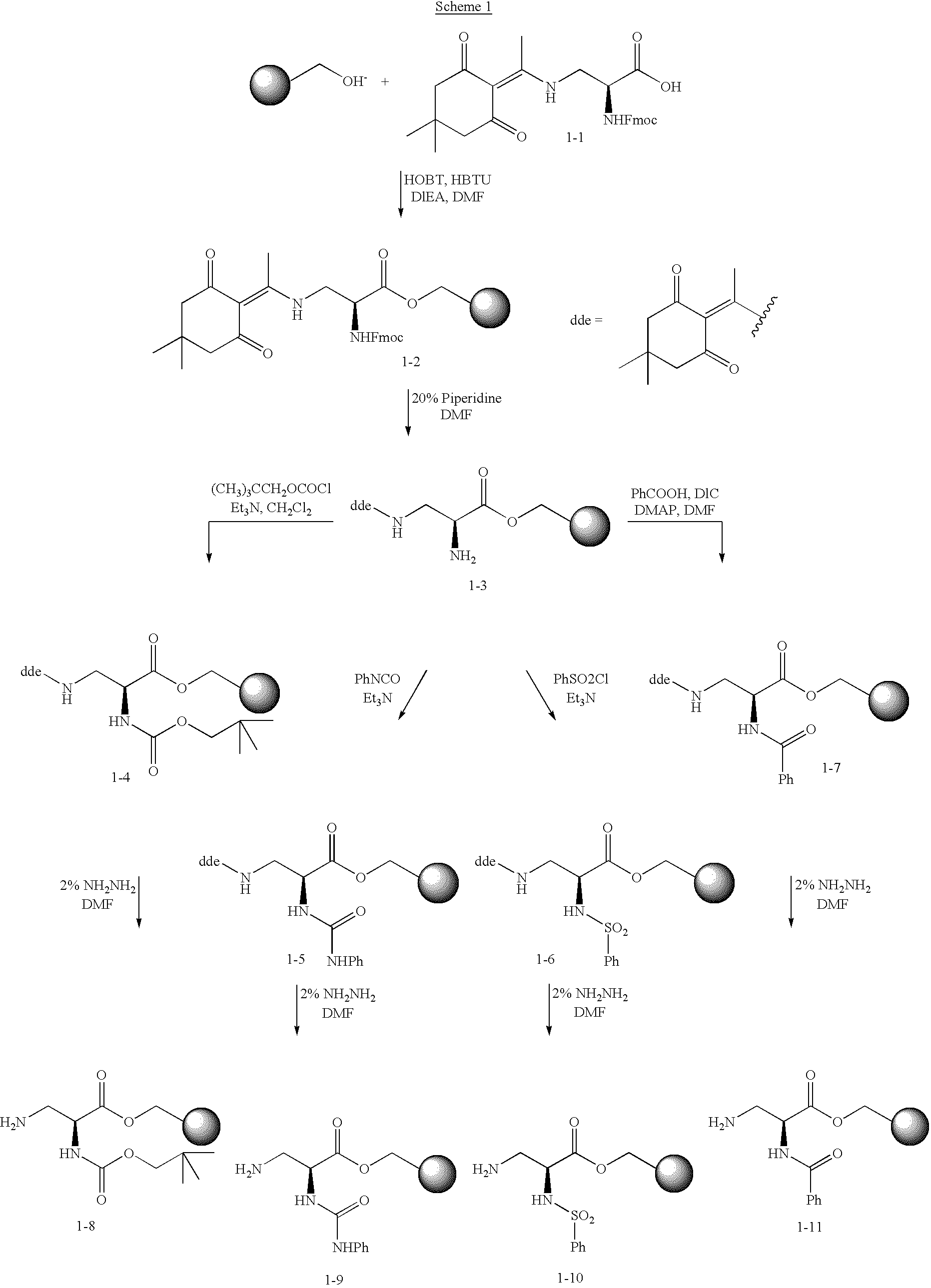

### 2(S)-(Fluorenylmethyloxycarbonyl)amino-3-(4,4-dimethyl-2.6dioxocyclohex-1-ylideneethyl)aminopropionic Acid on Wang Resin (1-2)

Wang resin (Wang, S. J. Am. Chem. Soc. 1973, 95, 1328-1333) (Advanced ChemTech 200-400 mesh, 1% crosslinked; loading: 0.92 mmol/g; 5g, 4.6 mmol) was swollen in N,N-dimethylformamide (DMF) (20 mL). A, solution of N-a-fmoc-N-b-1-(4,4-dimethyl-2,6-dioxocyclohex-1-ylidene)ethyl-L-diaminopropionic acid 1-1 (Fmoc-Dpr(Dde)-OH) (Nova Biochem) (4.513 g; 9.2 mmol) in DMF (30 mL) was treated with N-hydroxybenzotriazole (HOBT) (1.242 g; 9.2 mmol), 2-(1H-benzotriazole-1-yl)-1,1,3,3-tetramethyluronium hexafluorophosphate (HBTU) (3.487 g; 9.2 mmol) and N,N-diisopropylethylamine (DIEA) (3.2 mL; 18.4 mmol) and added to the resin. The mixture was shaken at room temperature for 8 h. The mixture was filtered and the resin was washed with DMF (3×40 mL), methanol (MeOH) (3×40 mL) and dichloromethane (DCM) (3×40 mL). The resin was dried in vacuo to give 6.956 g. Resin Loading: 0.8 mmol/g.

### 2-Amino-3-(4,4-dimethyl-2,6-dioxocylohex-1-ylideneethyl)aminopropionic Acid on Wang Resin (1-3)

The resin 1-2 (6.956 g) in DMF was treated with 20% piperidine in DMF (40 mL) for 10 min and filtered. Another 40 mL portion of 20% piperidine in DMF was added to the resin and shaken at room temperature for 20 min. The resin was filtered and washed with DMF (3×40 mL), MeOH (3×40 mL) and DCM (3×40 mL). The resin (1-3) was dried in vacuo.

### 2(S)-(2,2-Dimethyl-propoxycarbonylamino)-3-(4,4-dimethyl-2.6-dioxocylohex-1-ylideneethyl)amino-propionic acid on Wang Resin (1-4)

The resin 1-3 (925 mg; 0.75 mmol) was swollen in dichloromethane and treated with diisopropylethylamine (969 mg; 1.3 mL; 7.5 mmol) followed by neopentyl chloroformate (451.8 mg; 3 mmol). The reaction mixture was shaken at room temperature using orbital shaker (Thermolyne RotoMix Type 50800) for 18 h. The mixture were filtered and the resin was washed with dichloromethane (4×4 mL), methanol (4×mL) and dichloromethane (2×4 mL). The resins was dried under vacuum. A sample of the was removed and subjected to Kaiser Ninhydrin test. If the test showed the presence of free amine (resin turned blue) the coupling described above was repeated.

A sample of the resin was removed and subjected to cleavage with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. The product was characterized by HPLC: 4.28 min (82% @ 220 nm); MS: 383 (M+H)⁺.

The above reaction conditions were applied for synthesis of urea 1-5 and sulfonamide 1-6, using phenyl isocyanate and phenyl sulfonyl chloride, respectively, in the place of neopentyl chloroformate.

A number of chloroformates, isocyanates and sulfonyl chlorides were used in the above reaction.

2(S)-benzoylamino-3-(4,4-dimethyl-2,6-dioxocylohex-1-ylideneethyl)amino-propionic acid on Wang Resin (1-7)

The resin 1-3 (925 mg; 0.75 mmol) was washed with DMF to swell the resin. A solution of benzoic acid (183 mg; 1.5 mmole) in DMF was mixed with diisopropylcarbodiimide (192 mg; 0.25 mmole), hydroxybenzotriazole (228 mg; 1.5 mmole) and dimethylaminopyridine (18 mg; 1.5 mmole) and the mixture was added to the resin. The reaction mixture was shaken at room temperature for 16 h. The mixture was filtered and the resin was washed with dimethylformamide (4×4 mL), methanol (4×mL) and dichloromethane (4×4 mL). The resulting resin 1-7 was dried under vacuum. A sample of the resin was removed and subjected to Kaiser Ninhydrin test. If the test showed the presence of free amine (resin turned blue) the coupling described above was repeated.

Alternately, carboxylic acids were used in the above reaction in the place of benzoic acid.

### 3-Amino-2(S)-(2,2-dimethyl-propoxycarbonylamino)-propionic acid on Wang Resin (1-8

The resin 1-4 was shaken with a solution of 2% hydrazine in dimethylformamide (3 mL) for 5 min. at room temperature. The reaction mixture was filtered and an additional 3 mL of a solution of 2% hydrazine in dimethylformamide was added and the reaction mixture was shaken at room temperature for 5 min. The mixture was filtered and the resin was washed with dimethylformamide (4×4 mL), methanol (4×mL) and dichloromethane (4×4 mL). The resin was dried under vacuum. A sample of the resin was removed and subjected to Kaiser Ninhydrin test for the presence of free amine (resin turns blue).

A sample of the resin was removed and subjected to cleavage with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. The product was characterized by HPLC: 4.686 min (78% @ 220 nm); MS m

z 219 (M+H)⁺. Resin bound compounds 1-5, 1-6 and 1-7 were subjected to similar deprotection condition to afford the free amines 1-9, 1-10 and 1-11 respectively.

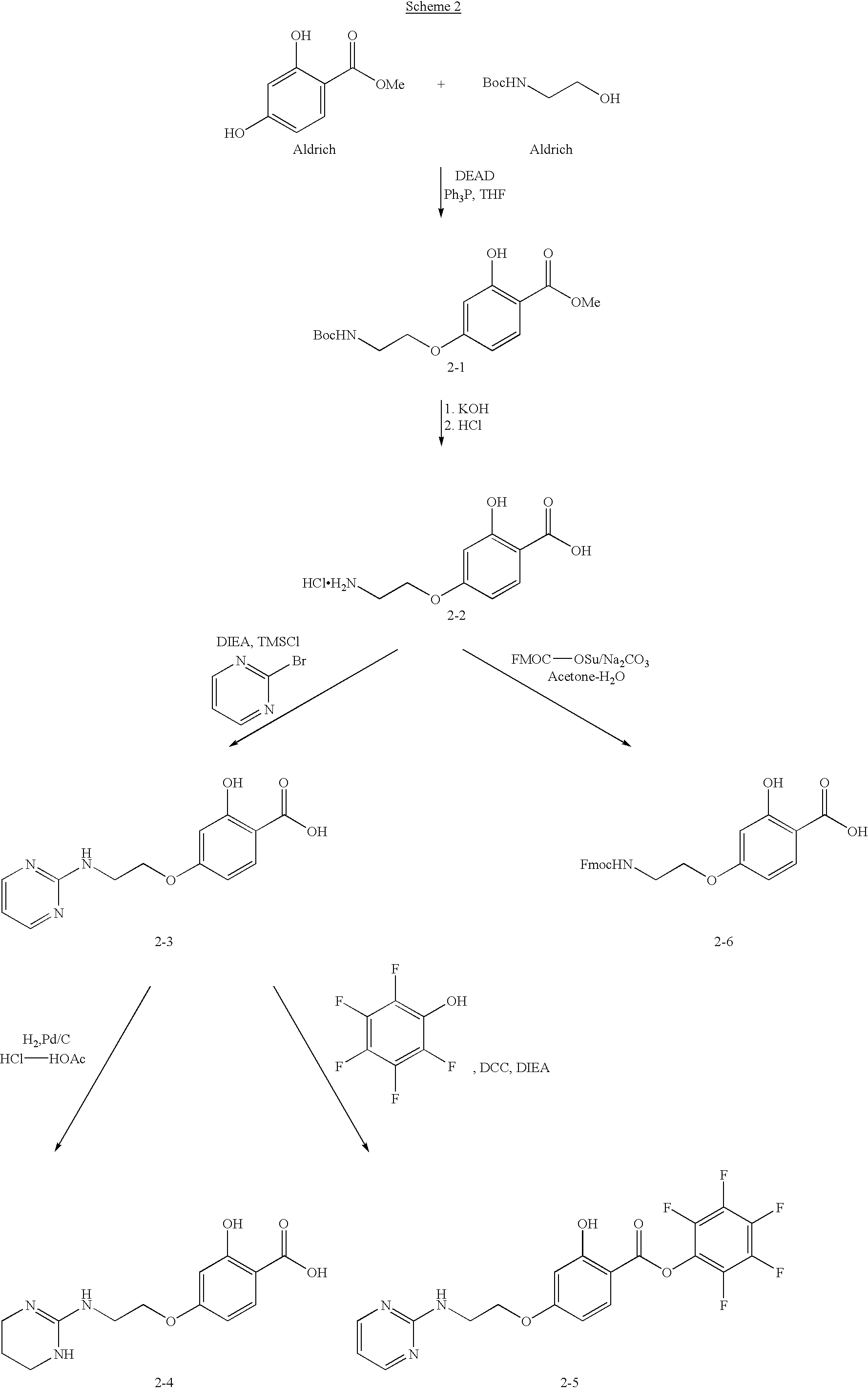

### Methyl 4-[2-N-(t-butoxycarbonyl)ethoxy]-2-hydroxy benzoate (2-1)

Methyl 2,4-dihydroxy benzoate (14.5 g, Aldrich), 2-(N-t-butoxycarbonyl)ethanol (13.9 g, Aldrich) and triphenyl phosphine (22.6 g, Aldrich) were combined in 350 mL of THF and cooled in ice under N₂ atmosphere. Diethyl diazodicarboxylate (DEAD, 15 g, Aldrich) was added, the ice bath removed and the reaction mixture allowed to stir at ambient temperature for 15 h. The solvent was removed on a rotary evaporator and the residue chromatographed on silica gel (300 g, Merck silica 60), elution with CH₂Cl₂ to give 18 g of methyl 4-[2-N-(t-butoxycarbonyl)ethoxy]-2-hydroxy benzoate, as a viscous oil. NMR (300 MHz, CDCl₃) δ 11.0 (s, 1H), 9.5 (d, J=8Hz, 1H), 6.4 (m, 2H), 5.0 (broad, 1H), 4.0 (t, J=5 Hz, 2H), 3.91 (s, 3H), 3.54 (m, 2H), 1.45 (s, 9H), MS (+ESI) m/z 334 (M+Na)⁺.

### 4-(2-Aminoethoxy)-2-hydroxybenzoic acid, hydrochloride (2-2)

Ester 2-1 (7.2 g) was treated with 5 eq. KOH (dissolved in minimum amount of water and equal volume of 1, 4-dioxane) at room temperature until TLC indicated complete absence of starting material (3-12 h). The reaction mixture was acidified (pH=6) with the addition of 1N HCl solution and extracted with ethyl acetate. The extract was washed with saturated aqueous brine solution, dried over MgSO₄, filtered and concentrated on the rotary evaporator. The crude product (5.34 g) was recrystallized from ether, then dissolved in 1, 4-dioxane and treated with an excess of anhydrous HCl (4M in dioxane, Aldrich). The mixture was allowed to stand at ambient temperature for 24 h. Volatile materials were removed in vacuo on the rotary evaporator to give 2-2 as a hydroscopic off-white solid. NMR (400 MHz, DMSO-d6) δ 13.6 (broad, 1H), 11.6 (broad, 1H), 8.3 (broad, 3H), 7.7 (d, J=9 Hz, 2H), 6.53 (m, 2H), 4.23 (t, J=5 Hz, 2H), 3.2 (s, broad, 2H).

### 2-Hydroxy-4-[2-(pyrimidine-2ylamino)ethoxy]benzoic acid (2-3)

A mixture of compound 2-2 (20 g), diissopropylethylamine (DIPEA, 74 mL), trimethylsilylchloride (TMSCl, 21.6 mL) and 2-bromopyrimidine (Lancaster, 13.5 g) were combined in 350 mL 1, 4-dioxane at room temperature, then brought to reflux under N₂ atmosphere. After 2 days, an additional 12 mL trimethylsilyl chloride was added, and the mixture continued at reflux for an additional 2 days (until TLC showed no stating material remained). The reaction mixture was cooled to ambient temperature, concentrated to dryness in vacuo on a rotary evaporator and the residue suspended in water. The heterogeneous mixture was refluxed briefly, allowed to cool to room temperature, the product collected on a vacuum filter and air dried to give 15.3 g of 2-3, as a tan powder. NMR (400 MHz, DMSO-d₆) δ 12 (very broad, 2H) 8.3 (d, J=5 Hz, 2H) 7.7 (d, J=9 Hz, 1H), 7.28 (t, J=6 Hz, 1H), 6.57 (t, J=5 Hz, 1H), 6.49 (m, 2H), 4.13 (t, J=6 Hz, 2H), 3.62 (q, 2H); MS (+ESI) m/z 276 (M+H)⁺; IR (KBr) ν (cm⁻¹) 3275, 3000, 1660, 1625.

### 2-Hydroxy-4-[2-(3,4,5,6-tetrahydropyrimidin-2ylamino)ethoxy]-benzoic acid (2-4).

Compound 2-3 (2 g) was combined with 10% Pd/C (0.5 g), acetic acid (100 mL) and concentrated hydrochloric acid (0.7 mL). The mixture was stirred at room temperature under an atmosphere of H₂ (balloon) for 2 days. Celite was added and the mixture stirred for 0.5 h, then filtered through a pad of celite with the aid of isopropanol. Volatile materials were removed on the rotary evaporator and the residue warmed with heptane (0.5 h, 100° C.) followed by concentration in vacuo to give 2-4 as a tan foam. NMR (400 MHz, DMSO-d₆) δ 12.9 (broad, 2H), 8.25 (s, broad, 2H), 7.85 (t, J=6 Hz, 1H), 7.66 (d, J=9 Hz, 1H), 6.48-6.41 (m, 2H), 4.07 (t, J=5 Hz, 2H), 3.56-3.50 (m, 2H), 3.22 (m, 2H, overlapping with H₂O peak), 1.79 (m, 2H); IR (KBr) n (cm⁻¹) 3450 (broad); MS (+ESI) In/z 280 (M +H)⁺.

### 2,3,4,5,6-Pentafluorophenyl 2-hydroxy-4-[2-(pyrimidine-2-ylamino)ethoxy]-benzoate (2-5)

Acid 2-3 (1.18 g; 4.3 mmol) in dioxane (40 mL) was treated with DIEA (1.5 mL; 8.6 mmol) and cooled to 0° C. Pentafluorophenol (3.16 g; 17.2 mmol) was added followed by dicyclohexyl carbodiimide. The reaction mixture was allowed to warm to room temperature and stirred for additional 16 h. The solid precipitated was filtered off and the mother liquor was concentrated to dryness and the residue was purified using silica column chromatography, eluted with 50% ethyl acetate in hexane to give 1.01 g of 2-5 as a white solid. NMR (300 MHz, CDCl3) δ 10.0 (s, 1H), 8.3 (d, J=5 Hz, 2H) 8.0 (d, J=9 Hz, 1H), 6.57 (t, J=5 Hz, 1H), 6.49 (in, 2H), 5.5 (t, J=3 Hz, 1H), 4.2 (t, J=6 Hz, 2H), 3.9 (q, 2H).

### 4-[(2-fluorenylmethyloxycarbonylamino)ethoxy]-2-hydroxybenzoic acid (2-6)

The Amino acid 2-2 (1.864 g; 8 mmol) was dissolved in 1:1 acetone-water (50 mL) containing sodium carbonate (1.696 g; 16 mmol). To the solution was added Fmoc-Osu (2.696 g; 8 mmol) in acetone (25 mL) dropwise at room temperature. The solution was stirred at room temperature for 18 h. The reaction mixture was concentrated and the residue was dissolved in water and extracted with ether (2×50 mL). The aqueous layer was cooled in an ice bath and acidified with 6N HCl to pH 3. The solid obtained was filtered and washed with water and dried under vacuo (3.22 g). NMR (300 MHz, DMSO-d6) δ 7.9 (d, 2H), 7.65-7.75 (m, 2H), 7.55 (t, 2H), 7.4 (t, 2H), 7.3 (t, 2H), 6.5 (m, 2H), 4.35 (d, 2H), 4.25 (t, 1H), 4.05 (t, 2H), 3.4 (t, 2H).

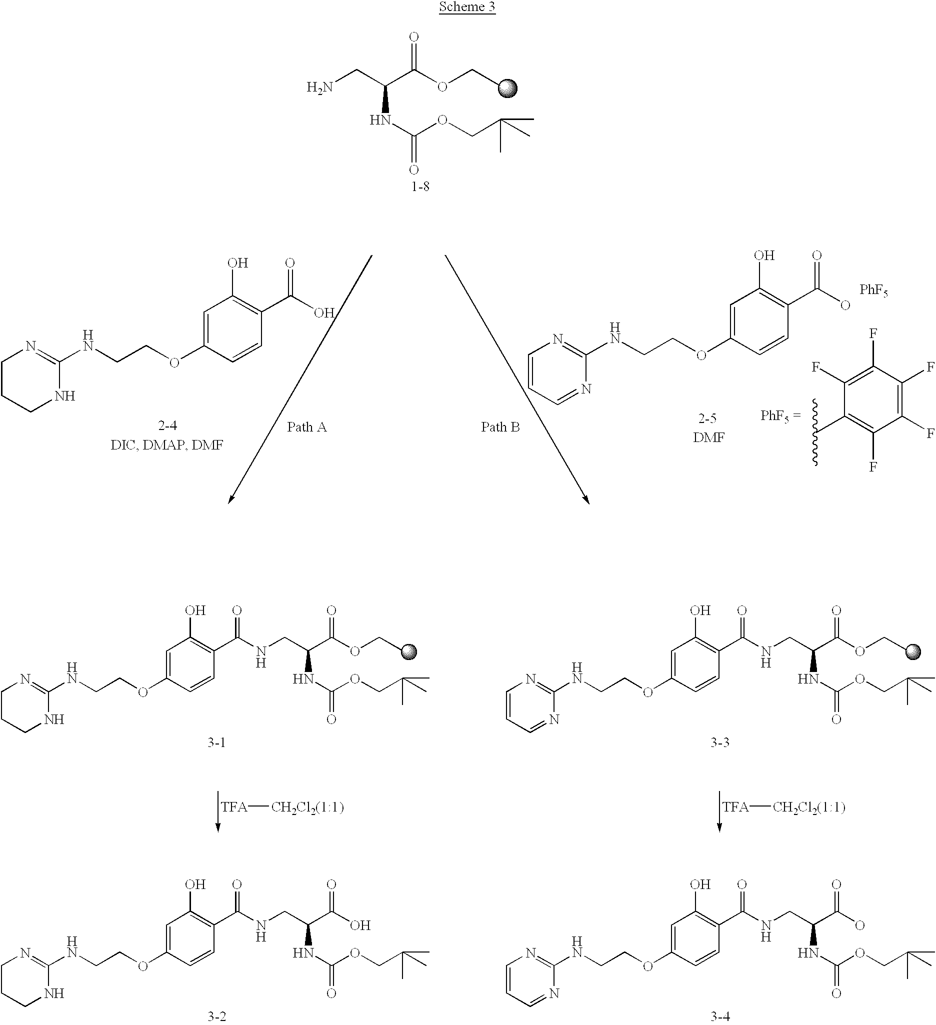

### EXAMPLE 1

### (2S)-3-({2-hydroxy-4-[2-(1,4,5,6-tetrahydropyrimidin-2-ylamino)ethoxy]benzoyl}amino)-2-{[(neopentyloxy)carbonyl]amino}propanoic acid (3-2)

### (2S)-3-(}2-hydroxy-4-[2-(1,4,5,6-tetrahydropyrimidin-2-ylamino)ethoxy]benzoyl}-amino)-2-{[(neopentyloxy)carbonyl]amino}propanoic acid on Wang Resin (3-1)

The resin 1-8 (100 mg) was washed with DMF to swell the resin. A solution of 2-hydroxy-4-[2-(3,4,5,6-tetrahydropyrimidin-2-ylamino)ethoxy]-benzoic acid 2-4 (70 mg; 0.25 mmole) in DMF was mixed with diisopropylcarbodiimide (32 mg; 0.25 mmole), hydroxybenzotriazole (38 mg; 0.25 mmole) and dimethylaminopyridine (3 mg; 0.025 mmole) and the mixture was added to the resin. The reaction mixture was shaken at room temperature for 16 h. The mixture was filtered and the resin was washed with dimethylformamide (4×4 mL), methanol (4×mL) and dichloromethane (4×4 mL). The resulting resin was dried under vacuum. A sample of the resin was removed and subjected to Kaiser Ninhydrin test. If the test showed the presence of free amine (resin turned blue) the coupling described above was repeated.

The resin 3-1 was treated with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. This crude product 3-2 was purified via preparative HPLC. NMR (400 MHz, MeOH-d4) δ 7.7 (d, J=7 Hz, 1H), 6.5 (m, 2H), 4.45 (q, 1H), 4.1 (t, 2H), 3.8-3.65 (m, 4H), 3.55 (t, 2H), 3.35 (t, 4H), 2.0 (m, 2H), 0.9 (s, 9H).

HR-MS FAB m/z for C₂₂H₃₃N₅O₇ calcd. 480.2458 (M⁺+1), obsd. 480.2431.

The following compounds were synthesized as described in the above Scheme 3 (Path A), using various resin bound carbamates in the place of (1-8). These compounds were characterized using LC and MS as shown in Table 1.

### EXAMPLE 2

(2S)-3-({2-hydroxy-4-[2-(1,4,5,6-tetrahydropyrimidin-2-ylamino)ethoxy]benzoyl}-amino)-2-[(methoxycarbonyl)amino]propanoic acid.

### EXAMPLE 3

(2S)-2-[(ethoxycarbonyl)amino]-3-({2-hydroxy-4-[2-(1,4,5,6-tetrahydropyrimidin-2ylamino)ethoxy]benzoyl}amino)propanoic acid.

### EXAMPLE 4

(2S)-3-({2-hydroxy-4-[2-(1,4,5,6-tetrahydropyrimidin-2-ylamino)ethoxy]benzoyl}-amino)-2-[(propoxycarbonyl)amino]propanoic acid.

### EXAMPLE 5

(2S)-3-({2-hydroxy-4-[2-(1,4,5,6-tetrahydropyrimidin-2-ylamino)ethoxy]benzoyl}-amino)-2-[(isopropoxycarbonyl)amino]propanoic acid.

### EXAMPLE 6

(2S)-2-{[(allyloxy)carbonyl]amino}-3-({2-hydroxy-4-[2-(1,4,5,6-tetrahydropyrimidin-2-ylamino)ethoxy]benzoyl}amino)propanoic acid.

### EXAMPLE 7

(2S)-2-{[(but-3-enyloxy)carbonyl]amino}-3-({2-hydroxy-4-[2-(1,4,5,6-tetrahydropyrimidin-2-ylamino)ethoxy]benzoyl}amino)propanoic acid.

### EXAMPLE 8

(2S)-2-{[(hexyloxy)carbonyl]amino}-3-({2-hydroxy-4-[2-(1,4,5,6-tetrahydropyrimidin-2-ylamino)ethoxy]benzoyl}amino)propanoic acid.

### EXAMPLE 9

(2S)-3-({2-hydroxy-4-[2-(1,4,5,6-tetrahydropyrimidin-2-ylamino)ethoxy]benzoyl}-amino)-2-{[(octyloxy)carbonyl]amino}propanoic acid.

### EXAMPLE 10

(2S)-3-({2-hydroxy-4-[2-(1,4,5,6-tetrahydropyrimidin-2-ylamino)ethoxy]-benzoyl}amino)-2-{[(2,2,2-trichloroethoxy)carbonyl]amino}propanoic acid.

### EXAMPLE 11

(2S)-3-({2-hydroxy-4-[2-(1,4,5,6-tetrahydropyrimidin-2-ylamino)ethoxy]-benzoyl}amino)-2-[(butoxycarbonyl)amino]propanoic acid.

### EXAMPLE 12

(2S)-3-({2-hydroxy-4-[2-(1,4,5,6-tetrahydropyrimidin-2-ylamino)ethoxy]benzoyl}-amino)-2-[(isobutoxycarbonyl)amino]propanoic acid.

### EXAMPLE 13

(2S)-3-({2-hydroxy-4-[2-(1,4,5,6-tetrahydropyrimidin-2-ylamino)ethoxy]benzoyl}-amino)-2-{[(prop-2-ynyloxy)carbonyl]amino}propanoic acid.

### EXAMPLE 14

(2S)-2-{[(benzyloxy)carbonyl]amino}-3-({2-hydroxy-4-[2-(1,4,5,6-tetrahydropyrimidin-2-ylamino)ethoxy]benzoyl}amino)propanoic acid.

TABLE 1

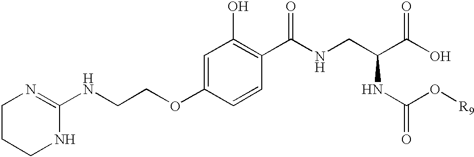

| | | LC @ 254 | | | | LC @ 254 | |
|---|---|---|---|---|---|---|---|
| Ex. | R9 | nm | (M + H)⁺ | Ex. | R9 | nm | (M + H)⁺ |
| 2 | Methyl | 2.92 min | 424 | 8 | n-Hexyl | 4.12 min | 494 |
| 3 | Ethyl | 3.09 min | 438 | 9 | n-Octyl | 4.62 min | 522 |
| 4 | n-Propyl | 3.30 min | 452 | 1 | (CH3)3CCH2 | 3.77 min | 480 |
| 5 | i-Propyl | 3.28 min | 452 | 10 | (CCl3)3CCH2 | 3.81 min | 542 |
| 6 | Allyl | 3.21 min | 450 | 11 | n-Butyl | 3.60 min | 466 |
| 7 | Homoallyl | 3.46 min | 463 | 12 | i-Butyl | 3.58 min | 466 |
| 13 | Propargyl | 3.18 min | 448 | 14 | Benzyl | 3.74 min | 500 |

The following compounds were synthesized as described in the above Scheme 3, (Path A) using various resin linked ureas 1-9 in the place of carbamate (1-8). These compounds were characterized using LC and MS as shown in Table 2.

### EXAMPLE 15

(2S)-2-{[(butylamino)carbonyl]amino}-3-({2-hydroxy-4-[2-(1,4,5,6-tetrahydropyrimidin-2-ylamino)ethoxy]benzoyl}amino)propanoic acid.

### EXAMPLE16

### (2S)-2-{[(hexylamino)carbonyl]amino}-3-({2-hydroxy-4-[2-(1,4,5,6-tetrahydropyrimidin-2-ylamino)ethoxy]benzoyl}amino)propanoic acid.

### EXAMPLE 17

(2S)-3-({2-hydroxy-4-[2-(1,4,5,6-tetrahydropyrimidin-2-ylamino)ethoxy]benzoyl}-amino)-2-{[(octylamino)carbonyl]amino}propanoic acid.

### EXAMPLE 18

(2S)-2-{[(allylamino)carbonyl]amino}-3-({2-hydroxy-4-[2-(1,4,5,6-tetrahydropyrimidin-2-ylamino)ethoxy]benzoyl}amino)propanoic acid.

### EXAMPLE 19

(2S)-2-{[(1-adamantylamino)carbonyl]amino}-3-({2-hydroxy-4-[2-(1,4,5,6-tetrahydropyrimidin-2-ylamino)ethoxy]benzoyl}amino)propanoic acid.

### EXAMPLE 20

(2S)-2-[(anilinocarbonyl)amino]-3-({2-hydroxy-4-[2-(1,4,5,6-tetrahydropyrimidin-2-ylamino)ethoxy]benzoyl}amino)propanoic acid.

### EXAMPLE 21

(2S)-2-{[(cyclohexylamino)carbonyl]amino}-3-({2-hydroxy-4-[2-(1,4,5,6-tetrahydropyrimidin-2-ylamino)ethoxy]benzoyl}amino)propanoic acid.

### EXAMPLE 22

(2S)-2-{[(benzylamino)carbonyl]amino}-3-({2-hydroxy-4-[2-(1,4,5,6-tetrahyrdopyrimidin-2-ylamino)ethoxy]benzoyl}amino)propanoic acid.

### EXAMPLE 23

(2S)-3-({2-hydroxy-4-[2-(1,4,5,6-tetrahydropyrimidin-2-ylamino)ethoxy]benzoyl}-amino)-2-[(4-toluidinocarbonyl)amino]propanoic acid.

### EXAMPLE 24

(2S)-3-({2-hydroxy-4-[2-(1,4,5,6-tetrahydropyrimidin-2-ylamino)ethoxy]benzoyl}-amino)-2-[(2-toluidinocarbonyl)amino]propanoic acid.

### EXAMPLE 25

(2S)-3-({2-hydroxy-4-[2-(1,4,5,6-tetrahydropyrimidin-2-ylamino)ethoxy]benzoyl}-amino)-2-{[(2-methoxyanilino)carbonyl]amino}propanoic acid.

### EXAMPLE 26

(2S)-3-(2-hydroxy-4-[2-(1,4,5,6-tetrahydropyrimidin-2-ylamino)ethoxy]benzoyl}-amino)-2-{[(4-methoxyanilino)carbonyl]amino}propanoic acid.

### EXAMPLE 27

(2S)-2-{[(2-chloroanilino)carbonyl]amino}-3-({2-hydroxy-4-[2-(1,4,5,6-tetrahydropyrimidin-2-ylamino)ethoxy]benzoyl}amino)propanoic acid.

### EXAMPLE 28

(2S)-2-{[(2-bromoanilino)carbonyl]amino}-3-({2-hydroxy-4-[2-(1,4,5,6-tetrahydropyrimidin-2-ylamino)ethoxy]benzoyl}amino)propanoic acid.

### EXAMPLE 29

(2S)-2-{[([1,1′-biphenyl]-2-ylamino)carbonyl]amino}-3-({2-hydroxy-4-[2-(1,4,5,6-tetrahydropyrimidin-2-ylamino)ethoxy]benzoyl}amino)propanoic acid.

### EXAMPLE 30

(2S)-2-{[(4-chloroanilino)carbonyl]amino}-3-({2-hydroxy-4-[2-(1,4,5,6-tetrahydropyrimidin-2-ylamino)ethoxy]benzoyl}amino)propanoic acid.

### EXAMPLE 31

(2S)-3-({2-hydroxy-4-[2-(1,4,5,6-tetrahydropyrimidin-2-ylamino)ethoxy]benzoyl}-amino)-2-{[(1-naphthylamino)carbonyl]amino}propanoic acid.

### EXAMPLE 32

(2S)-3-({2-hydroxy-4-[2-(1,4,5,6-tetrahydropyrimidin-2-ylamino)ethoxy]benzoyl}-amino)-2-({[(2-phenylethyl)amino]carbonyl}amino)propanoic acid.

TABLE 2

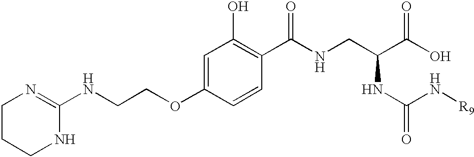

| | | | | | | LC @ 254 | |
|---|---|---|---|---|---|---|---|
| Ex. | R9 | LC @ 254 nm | (M + H)⁺ | Ex. | R9 | nm | (M + H)⁺ |
| 15 | 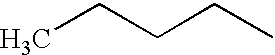 | 3.30 min | 465 | 24 | 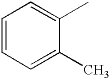 | 3.45 min | 499 |
| 16 | 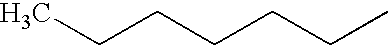 | 3.77 min | 493 | 25 | 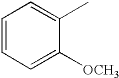 | 3.50 min | 515 |
| 17 | 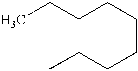 | 4.31 min | 521 | 26 | 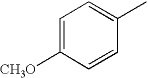 | 3.39 min | 515 |
| 18 | 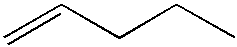 | 3.02 min | 449 | 27 | 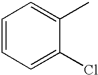 | 3.60 min | 521 |
| 19 | 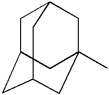 | 4.00 min | 543 | 28 | 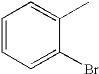 | 3.60 min | 565 |
| 20 | 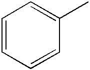 | 3.42 min | 485 | 29 | 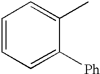 | 3.95 min | 561 |
| 21 | 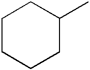 | 3.45 min | 491 | 30 | 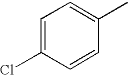 | 3.79 min | 521 |
| 22 | 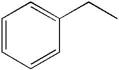 | 3.43 min | 499 | 31 | 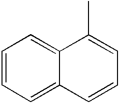 | 3.67 min | 535 |
| 23 | 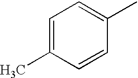 | 3.62 min | 499 | 32 | 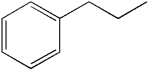 | 3.56 min | 513 |

The following compounds were synthesized as described in the above Scheme 3, (Path A) using various resin linked amides 1-11 in the place of carbamate (1-8). These compounds were characterized using LC and MS as shown in Table 3.

### EXAMPLE 33

(2S)-3-({2-hydroxy-4-[2-(1,4,5,6-tetrahydropyrimidin-2-ylamino)ethoxy]benzoyl}-amino)-2-(isobutyrylamino)propanoic acid.

### EXAMPLE 34

(2S)-2-(hexanoylamino)-3-({2-hydroxy-4-[2-(1,4,5,6-tetrahydropyrimidin-2-ylamino)ethoxy]benzoyl}amino)propanoic acid.

### EXAMPLE 35

(2S)-3-({2-hydroxy-4-[2-(1,4,5,6-tetrahydropyrimidin-2-ylamino)ethoxy]benzoyl}-amino)-2-(pentanoylamino)propanoic acid.

### EXAMPLE 36

(2S)-2-[(3,3-dimethylbutanoyl)amino]-3-({2-hydroxy-4-[2-(1,4,5,6-tetrahydropyrimidin-2-ylamino)ethoxy]benzoyl}amino)propanoic acid.

### EXAMPLE 37

(2S)-2-[(cyclohexylcarbonyl)amino]-3-({2-hydroxy-4-[2-(1,4,5,6-tetrahydropyrimidin-2-ylamino)ethoxy]benzoyl}amino)propanoic acid.

### EXAMPLE 38

(2S)-3-({2-hydroxy-4-[2-(1,4,5,6-tetrahydropyrimidin-2-ylamino)ethoxy]benzoyl}-amino)-2-[(2-phenylacetyl)amino]propanoic acid.

### EXAMPLE 39

(2S)-3-({2-hydroxy-4-[2-(1,4,5,6-tetrahydropyrimidin-2-ylamino)ethoxy]benzoyl}-amino)-2-[(3-phenylpropanoyl)amino]propanoic acid.

### EXAMPLE 40

(2S)-2-[(2-cyclohexylacetyl)amino]-3-({2-hydroxy-4-[2-(1,4,5,6-tetrahydropyrimidin-2-ylamino)ethoxy]benzoyl}amino)propanoic acid.

### EXAMPLE 41

(2S)-3-({2-hydroxy-4-[2-(1,4,5,6-tetrahydropyrimidin-2-ylamino)ethoxy]benzoyl}-amino)-2-{[(E)-3-phenylprop-2-enoyl]amino}propanoic acid.

### EXAMPLE 42

(2S)-2-[(2-chlorobenzoyl)amino]-3-({2-hydroxy-4-[2-(1,4,5,6-tetrahydropyrimidin-2-ylamino)ethoxy]benzoyl}amino)propanoic acid.

### EXAMPLE 43

(2S)-3-({2-hydroxy-4-[2-(1,4,5,6-tetrahydropyrimidin-2 -ylamino)ethoxy]benzoyl}amino)-2-[(2-methylbenzoyl)amino]propanoic acid.

### EXAMPLE 44

(2S)-3-({2-hydroxy-4-[2-(1,4,5,6-tetrahydropyrimidin-2-ylamino)ethoxy]benzoyl}-amino)-2-[(2-methoxybenzoyl)amino]propanoic acid.

### EXAMPLE 45

(2S)-2-[(4-chlorobenzoyl)amino]-3-({2-hydroxy-4-[2-(1,4,5,6-tetrahydropyrimidin-2-ylamino)ethoxy]benzoyl}amino)propanoic acid.

### EXAMPLE 46

(2S)-3-({2-hydroxy-4-[2-(1,4,5,6-tetrahydropyrimidin-2-ylamino)ethoxy]benzoyl}-amino)-2-[(4-methylbenzoyl)amino]propanoic acid.

### EXAMPLE 47

(2S)-3-({2-hydroxy-4-[2-(1,4,5,6-tetrahydropyrimidin-2-ylamino)ethoxy]benzoyl}-amino)-2-[(4-methoxybenzoyl)amino]propanoic acid.

### EXAMPLE 48

(2S)-2-[(2,5-dimethyl-3-furoyl)amino]-3-({2-hydroxy-4-[2-(1,4,5,6-tetrahydropyrimidin-2-ylamino)ethoxy]benzoyl}amino)propanoic acid.

### EXAMPLE 49

(2S)-2-[(2-bromobenzoyl)amino]-3-({2-hydroxy-4-[2-(1,4,5,6-tetrahydropyrimidin-2-ylamino)ethoxy]benzoyl}amino)propanoic acid.

### EXAMPLE 50

(2S)-2-[(4-bromobenzoyl)amino]-3-({2-hydroxy-4-[2-(1,4,5,6-tetrahydropyrimidin-2-ylamino)ethoxy]benzoyl}amino)propanoic acid.

### EXAMPLE 51

(2S)-2-[(2,3-dimethylbenzoyl)amino]-3-({2-hydroxy-4-[2-(1,4,5,6-tetrahydropyrimidin-2-ylamino)ethoxy]benzoyl}amino)propanoic acid.

### EXAMPLE 52

(2S)-2-[(3-chlorobenzoyl)amino]-3-({2-hydroxy-4-[2-(1,4,5,6-tetrahydropyrimidin-2-ylamino)ethoxy]benzoyl}amino)propanoic acid.

TABLE 3

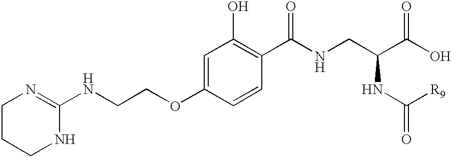

| Ex. | R9 | LC @ 254 nm | (M + H)⁺ | Ex. | R9 | LC @ 254 nm | (M + H)⁺ |
|---|---|---|---|---|---|---|---|
| 33 | 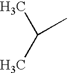 | 3.03 min | 436 | 43 | 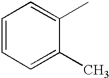 | 3.42 min | 484 |
| 34 | 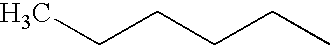 | 3.46 min | 464 | 44 | 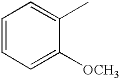 | 3.48 min | 500 |
| 35 | 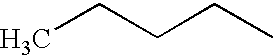 | 3.24 min | 450 | 45 | 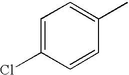 | 3.70 min | 504 |
| 36 | 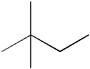 | 3.37 min | 464 | 46 | 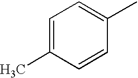 | 3.57 min | 484 |
| 37 | 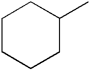 | 3.49 min | 476 | 47 | 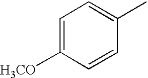 | 3.44 min | 500 |
| 38 | 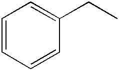 | 3.35 min | 484 | 48 | 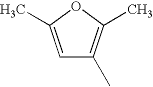 | 3.59 min | 488 |
| 39 | 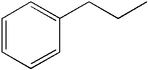 | 3.54 min | 498 | 49 | 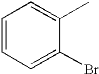 | 3.39 min | 548 |
| 40 | 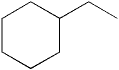 | 3.62 min | 490 | 50 | 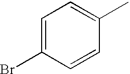 | 3.76 min | 548 |
| 41 | 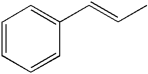 | 3.66 min | 496 | 51 | 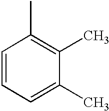 | 3.58 min | 498 |
| 42 | 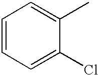 | 3.36 min | 504 | 52 | 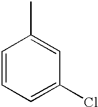 | 3.70 min | 504 |

### EXAMPLE 53

### (2S)-3-({2-hydroxy-4-[2-(pyrimidin-2-ylamino)ethoxy]benzoyl}-amino)-2-{[(neopentyloxy)carbonyl]amino}propanoic acid (3-4)

### (2S)-3-({2-hydroxy-4-[2-(pyrimidin-2-ylamino)ethoxy]benzoyl}amino)-2-{[(neopentyloxy)carbonyl]amino}propanoic acid on Wang Resin (3-3)

The resin 1-8 (100 mg) was washed with DMF to swell the resin and was treated with a solution of 2,3,4,5,6-pentafluorophenyl 2-hydroxy-4-[2-(pyrimidine-2-ylamino)ethoxy]-benzoate 2-5 (110 mg; 0.25 mmole) in DMF. The reaction mixture was shaken at room temperature for 16 h. The mixture was filtered and the resin was washed with dimethylformamide (4×4 mL), methanol (4×mL) and dichloromethane (4×4 mL). The resulting resin 3-3 was dried under vacuum. A sample of the resin was removed and subjected to Kaiser Ninhydrin test. If the test showed the presence of free amine (resin turned blue) the coupling described above was repeated.

The resin 3-3 was treated with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. This crude product 3-4 was purified via preparative HPLC. 3.907 min (78% @ 220 nm); MS m/z 476 (M+H)⁺.

The following compounds were synthesized as described in the above Scheme 3 (Path B), using various resin bound carbamates in the place of (1-8). These compounds were characterized using LC and MS as shown in Table 4.

### EXAMPLE 54

(2S)-3-({2-hydroxy-4-[2-(pyrimidin-2-ylamino)ethoxy]benzoyl}amino)-2-[(phenoxycarbonyl)amino]propanoic acid.

### EXAMPLE 55

(2S)-2-{[(benzyloxy)carbonyl]amino}-3-({2-hydroxy-4-[2-(pyrimidin-2-ylamino)ethoxy]benzoyl}amino)propanoic acid.

### EXAMPLE 56

(2S)-3-({2-hydroxy-4-[2-(pyrimidin-2-ylamino)ethoxy]benzoyl}amino)-2-[(isobutoxycarbonyl)amino]propanoic acid.

### EXAMPLE 57

(2S)-3-({2-hydroxy-4-[2-(pyrimidin-2-ylamino)ethoxy]benzoyl}amino)-2-{[(4-methoxyphenoxy)carbonyl]amino}propanoic acid.

### EXAMPLE 58

(2S)-3-({2-hydroxy-4-[2-(pyrimidin-2-ylamino)ethoxy]benzoyl}amino)-2-{[(octyloxy)carbonyl]amino}propanoic acid.

### EXAMPLE 59

(2S)-2-[(butoxycarbonyl)amino]-3-({2-hydroxy-4-[2-(pyrimidin-2-ylamino)ethoxy]benzoyl}amino)propanoic acid.

### EXAMPLE 60

(2S)-3-({2-hydroxy-4-[2-(pyrimidin-2-ylamino)ethoxy]benzoyl}amino)-2-{[(2,2,2-trichloroethoxy)carbonyl]amino}propanoic acid.

### EXAMPLE 61

(2S)-3-({2-hydroxy-4-[2-(pyrimidin-2-ylamino)ethoxy]benzoyl}amino)-2-({[(4-nitrobenzyl)oxy]carbonyl}amino)propanoic acid.

### EXAMPLE 62

(2S)-2-{[(hexyloxy)carbonyl]amino}-3-({2-hydroxy-4-[2-(pyrimidin-2-ylamino)-ethoxy]benzoyl}amino)propanoic acid.

### EXAMPLE 63

(2S)-3-({2-hydroxy-4-[2-(pyrimidin-2-ylamino)ethoxy]benzoyl}amino)-2-{[(prop-2-ynyloxy)carbonyl]amino}propanoic acid.

### EXAMPLE 64

(2S)-3-({2-hydroxy-4-[2-(pyrimidin-2-ylamino)ethoxy]benzoyl}amino)-2-{[(4-methylphenoxy)carbonyl]amino}propanoic acid.

### EXAMPLE 65

(2S)-3-({2-hydroxy-4-[2-(pyrimidin-2-ylamino)ethoxy]benzoyl}amino)-2-[(methoxycarbonyl)amino]propanoic acid.

### EXAMPLE 66

(2S)-2-[(ethoxycarbonyl)amino]-3-({2-hydroxy-4-[2-(pyrimidin-2-ylamino)-ethoxy]benzoyl}amino)propanoic acid.

### EXAMPLE 67

(2S)-3-({2-hydroxy-4-[2-(pyrimidin-2-ylamino)ethoxy]benzoyl}amino)-2-[(propoxycarbonyl)amino]propanoic acid.

### EXAMPLE 68

(2S)-3-({2-hydroxy-4-[2-(pyrimidin-2-ylamino)ethoxy]benzoyl}amino)-2-[(isopropoxycarbonyl)amino]propanoic acid.

### EXAMPLE 69

(2S)-2-{[(allyloxy)carbonyl]amino}-3-({2-hydroxy-4-[2-(pyrimidin-2-ylamino)ethoxy]benzoyl}amino)propanoic acid.

### EXAMPLE 70

(2S)-2-{[(but-3-enyloxy)carbonyl]amino}-3-({2-hydroxy-4-[2-(pyrimidin-2-ylamino)ethoxy]benzoyl}amino)propanoic acid.

TABLE 4

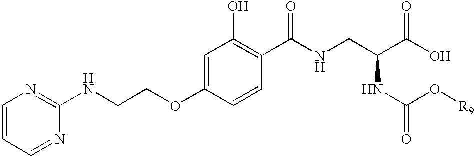

| Ex. | R9 | LC @ 254 nm | (M + H)⁺ | Ex. | R9 | LC @ 254 nm | (M + H)⁺ |
|---|---|---|---|---|---|---|---|
| 54 | Phenyl | 3.77 min | 481 | 62 | n-Hexyl | 4.26 min | 490 |
| 55 | Benzyl | 3.88 min | 495 | 63 | Propargyl | 3.30 min | 444 |
| 56 | i-Butyl | 3.73 min | 461 | 64 | p-Me-Phenyl | 3.94 v | 496 |
| 57 | p-OMe-phenyl | 3.75 min | 511 | 65 | Methyl | 3.06 v | 420 |
| 58 | Octyl | 4.79 min | 517 | 66 | Ethyl | 3.26 min | 434 |
| 59 | n-Butyl | 3.77 min | 462 | 67 | n-Propyl | 3.48 v | 448 |
| 60 | CCl3CH2 | 3.94 min | 538 | 68 | i-Propyl | 3.46 v | 448 |
| 53 | neopentyl | 3.90 min | 476 | 69 | Allyl | 3.40 min | 446 |
| 61 | p-NO2-Benzyl | 3.80 min | 541 | 70 | Homoallyl | 3.58 min | 460 |

The following compounds were synthesized as described in the above Scheme 3 (Path sing various resin bound ureas 1-9 in the place of (1-8). These compounds were characterized using LC and MS as shown in Table 5.

### EXAMPLE 71

(2S)-2-[(anilinocarbonyl)amino]-3-({2-hydroxy-4-[2-(pyrimidin-2-ylamino)ethoxy]-benzoyl}amino)propanoic acid

### EXAMPLE 72

(2S)-2-{[(tert-butylamino)carbonyl]amino}-3-({2-hydroxy-4-[2-(pyrimidin-2-ylamino)ethoxy]benzoyl}amino)propanoic acid.

### EXAMPLE 73

(2S)-2-{[(butylamino)carbonyl]amino}-3-({2-hydroxy-4-[2-(pyrimidin-2-ylamino)ethoxy]benzoyl}amino)propanoic acid.

### EXAMPLE 74

(2S)-3-({2-hydroxy-4-[2-(pyrimidin-2-ylamino)ethoxy]benzoyl3 amino)-2-{[(4-methoxyanilino)carbonyl]amino}propanoic acid

### EXAMPLE 75

(2S)-2-{[(2-ethylanilino)carbonyl]amino}-3-({2-hydroxy-4-[2-(pyrimidin-2-ylamino)ethoxy]benzoyl}amino)propanoic acid.

### EXAMPLE 76

(2S)-2-1 [(allylamino)carbonyl]amino}-3-({2-hydroxy-4-[2-(pyrimidin-2-ylamino)-ethoxy]benzoyl}amino)propanoic acid

### EXAMPLE 77

(2S)-2-{[(2,4-dichloroanilino)carbonyl]amino}-3-({2-hydroxy-4-[2-(pyrimidin-2-ylamino)ethoxy]benzoyl}amino)propanoic acid.

### EXAMPLE 78

(2S)-3-({2-hydroxy-4-[2-(pyrimidin-2-ylamino)ethoxy]benzoyl}amino)-2-[(2-toluidinocarbonyl)amino]propanoic acid.

### EXAMPLE 79

(2S)-3-({2-hydroxy-4-[2-(pyrimidin-2-ylamino)ethoxy]benzoyl}amino)-2-{[(2-methoxyanilino)carbonyl]amino}propanoic acid.

### EXAMPLE 80

(2S)-2-{[(2-chloroanilino)carbonyl]amino}-3-({2-hydroxy-4-[2-(pyrimidin-2-ylamino)ethoxy]benzoyl}amino)propanoic acid.

### EXAMPLE 81

(2S)-2-{[(2-bromoanilino)carbonyl]amino}-3-({2-hydroxy-4-[2-(pyrimidin-2-ylamino)ethoxy]benzoyl}amino)propanoic acid.

### EXAMPLE 82

(2S)-2-{[([1,1′-biphenyl]-2-ylamino)carbonyl]amino}-3-({2-hydroxy-4-[2-(pyrimidin-2-ylamino)ethoxy]benzoyl}amino)propanoic acid.

### EXAMPLE 83

(2S)-3-({2-hydroxy-4-[2-(pyrimidin-2-ylamino)ethoxy]benzoyl}amino)-2-[(4-toluidinocarbonyl)amino]propanoic acid.

### EXAMPLE 84

(2S)-3-({2-hydroxy-4-[2-(pyrimidin-2-ylamino)ethoxy]benzoyl}amino)-2-({[4-(trifluoromethyl)anilino]carbonyl}amino)propanoic acid.

### EXAMPLE 85

(2S)-3-({2-hydroxy-4-[2-(pyrimidin-2-ylamino)ethoxy]benzoyl}amino)-2-({[4-(trifluoromethoxy)anilino]carbonyl}amino)propanoic acid.

### EXAMPLE 86

(2S)-2-{[(4-chloroanilino)carbonyl]amino}-3-({2-hydroxy-4-[2-(pyrimidin-2-ylamino)ethoxy]benzoyl}amino)propanoic acid.

### EXAMPLE 87

(2S)-2-{[(4-fluoroanilino)carbonyl]amino}-3-({2-hydroxy-4-[2-(pyrimidin-2-ylamino)ethoxy]benzoyl}amino)propanoic acid.

### EXAMPLE 88

(2S)-2-{[(4-acetylanilino)carbonyl]amino}-3-({2-hydroxy-4-[2-(pyrimidin-2-ylamino)ethoxy]benzoyl}amino)propanoic acid.

### EXAMPLE 89

(2S)-2-({[4-(ethoxycarbonyl)anilino]carbonyl}amino)-3-({2-hydroxy-4-[2-(pyrimidin-2-ylamino)ethoxy]benzoyl}amino)propanoic acid.

### EXAMPLE 90

(2S)-2-{[(cyclohexylamino)carbonyl]amino}-3-({2-hydroxy-4-[2-(pyrimidin-2-ylamino)ethoxy]benzoyl}amino)propanoic acid.

### EXAMPLE 91

(2S)-3-({2-hydroxy-4-[2-(pyrimidin-2-ylamino)ethoxy]benzoyl}amino)-2-{[1-naphthylamino)carbonyl]amino}propanoic acid.

### EXAMPLE 92

(2S)-2-{[(benzylamino)carbonyl]amino}-3-({2-hydroxy-4-[2-(pyrimidin-2-ylamino)ethoxy]benzoyl}amino)propanoic acid.

### EXAMPLE 93

(2S)-3-({2-hydroxy-4-[2-(pyrimidin-2-ylamino)ethoxy]benzoyl}amino)-2-({[(2phenylethyl)amino]carbonyl}amino)propanoic acid

### EXAMPLE 94

(2S)-3-({2-hydroxy-4-[2-(pyrimidin-2-ylamino)ethoxy]benzoyl}amino)-2-{[(octylamino)carbonyl]amino}propanoic acid

TABLE 5

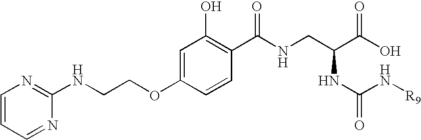

| Ex. | R9 | LC @ 254 nm | (M + H)⁺ |
|---|---|---|---|
| 71 | 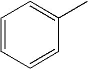 | 3.70 min | 480 |
| 72 | 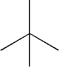 | 3.45 min | 460 |
| 73 | 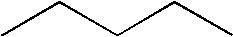 | 3.50 min | 460 |
| 74 | 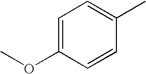 | 3.56 min | 510 |
| 75 | 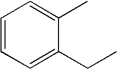 | 3.81 min | 508 |
| 76 | 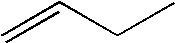 | 3.13 min | 444 |
| 77 | 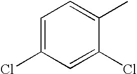 | 4.19 min | 549 |
| 78 | 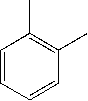 | 3.63 min | 495 |
| 79 | 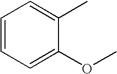 | 3.68 min | 511 |
| 80 | 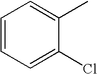 | 3.78 min | 515 |
| 81 | 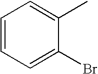 | 3.80 min | 559 |
| 82 | 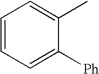 | 4.13 min | 557 |
| 83 | 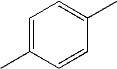 | 3.79 min | 495 |
| 84 | 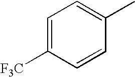 | 4.22 min | 549 |
| 85 | 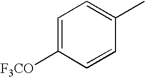 | 4.27 min | 565 |
| 86 | 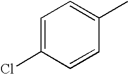 | 3.96 min | 515 |
| 87 | 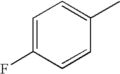 | 3.68 min | 499 |
| 88 | 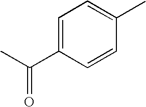 | 3.50 min | 523 |
| 89 | 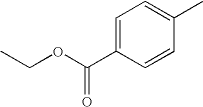 | 3.92 min | 553 |
| 90 | 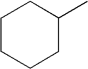 | 3.66 min | 487 |
| 91 | 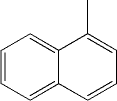 | 3.86 min | 487 |
| 92 | 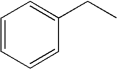 | 3.55 min | 495 |
| 93 | 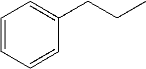 | 3.70 min | 509 |
| 94 | 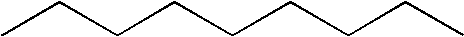 | 4.56 min | 517 |

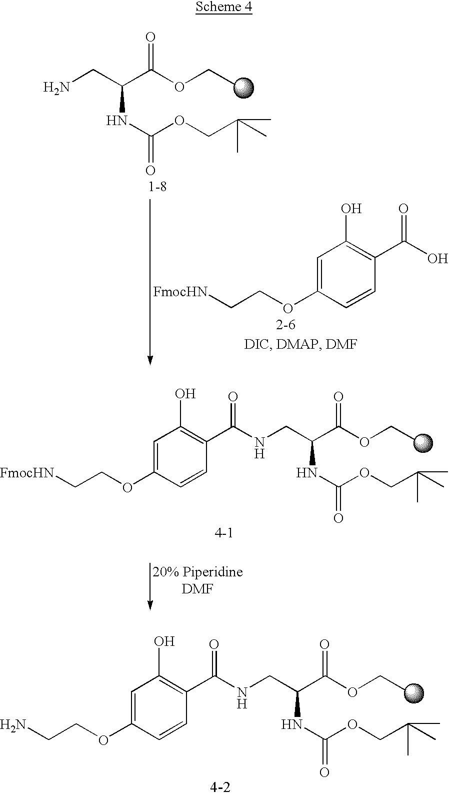

### 2(S)-(2,2-dimethyl-propoxycarbonylamino)-3-{[2-Hydroxy-4-(2-fluorenylmethyloxy-carbonylamino)ethoxy]benzoylamino}-propionic acid on Wang Resin (4-1)

The resin 1-8 was washed with DMF to swell the resin. A solution of 4-[(2-fluorenylmethyloxycarbonylamino)ethoxy]-2-hydroxybenzoic acid (2-6) (628.5 mg; 1.5 mmole) in DMF was mixed with diisopropylcarbodiimide (189 mg; 1.5 mmole), hydroxybenzotriazole (202.5 mg; 1.5 mmole) and dimethylaminopyridine (18.33 mg; 0.15 mmole) and the mixture was added to the resin. The reaction mixture was shaken at room temperature for 16 h. The mixture was filtered and the resin was washed with dimethylformamide (4×4 mL), methanol (4×mL) and dichloromethane (4×4 mL). The resin was dried under vacuum. A sample of the resin was removed and subjected to Kaiser Ninhydrin test. If the test showed the presence of free amine (resin turned blue) the coupling described above was repeated.

### 2(S)-(2,2-dimethyl-propoxycarbonylamino)-3-[2-Hydroxy-4-(2-aminoethoxy)benzoyl-amino-propionic acid on Wang Resin (4-2)

The resin 4-1 was shaken with a solution of 20% piperidine in DMF (5 mL) for 10 min and filtered. Another 5 mL portion of 20% piperidine in DMF was added and shaken at room temperature for 20 min. The resin was filtered and washed with DMF (3×40 mL), MeOH (3×40 mL) and DCM (3×40 mL). The resin were dried under vacuum.

A sample of the resin was removed and subjected to cleavage with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. The product was characterized by HPLC: 3.35 min (70% @ 220 nm); MS m/z 398 (M+H)⁺.

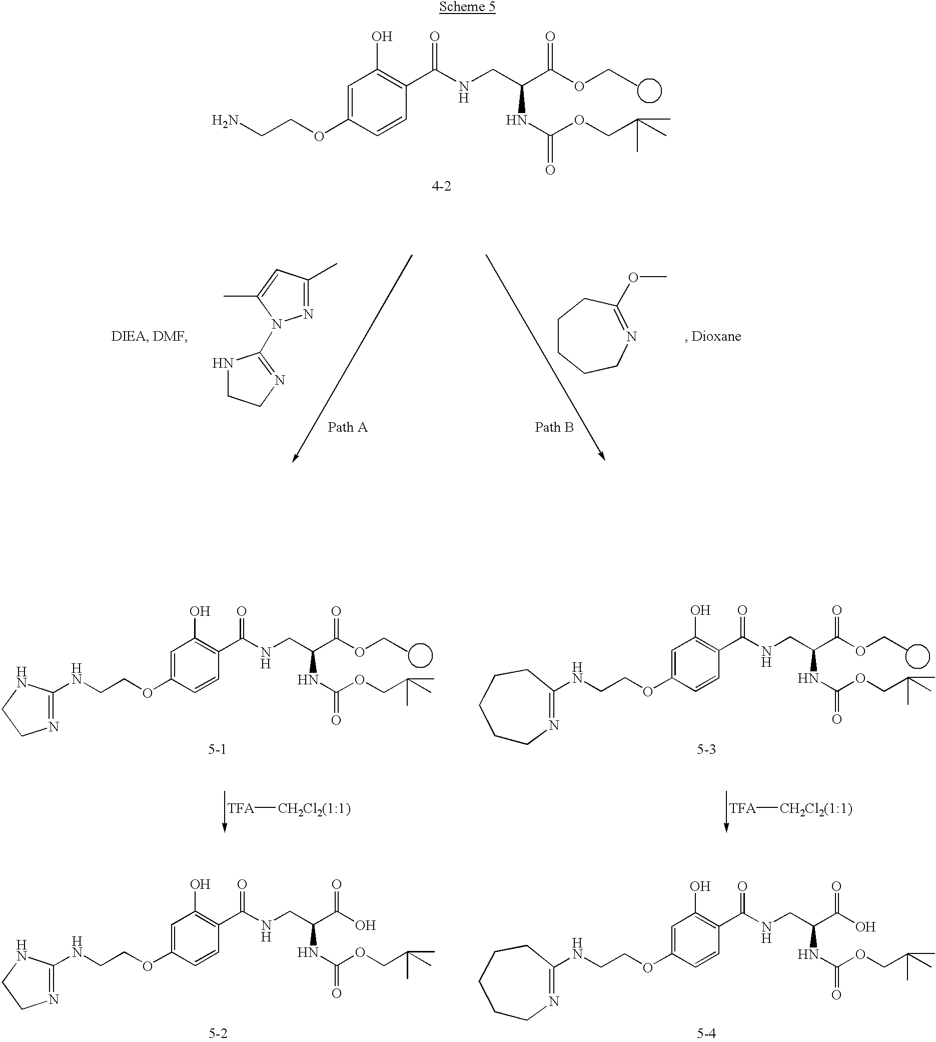

### EXAMPLE 95

### (2S)-3-(14-[2-(4,5-dihydro-1H-imidazol-2-ylamino)ethoxy]-2-hydroxybenzoyl}amino)-2-{[(neopentyloxy)carbonyl]amino}propanoic acid (5-2)

### (2S)-3-({4-[2-(4,5-dihydro-1H-imidazol-2-ylamino)ethoxy]-2-hydroxybenzoyl}-amino)-2-{[(neopentyloxy)carbonyl]amino}propanoic acid on Wang Resin (5-1)

The resin 4.2 (100 mg; 0.1 mmole) was swollen in DMF. To the resin was added a solution of 2-(3,5-dimethylpyrazolyl)-4,5-dihydroimdazole hydrobromide (123 mg; 0.5 mmole) in DMF (1.5 mL) followed by diisopropylamine (0.15 mL; 1 mmole). The reaction vessel was shaken at 60° C. for 18 h. The mixture was filtered and the resin was washed with dimethylformamide (4×4 mL), methanol (4×mL) and dichloromethane (4×4 mL). The resin was dried under vacuum. A sample of the resin was removed and subjected to Kaiser Ninhydrin test. If the test showed the presence of free amine (resin turned blue) the coupling described above was repeated.

The resin 5-1 was cleaved by treatment with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. This crude product 5-2 was purified via preparative HPLC. NMR (300 MHz, MeOH-d4) 67 7.7 (d, J=7 Hz, 1H), 6.5 (m, 2H), 4.5 (q, 1H), 4.2 (t, 2H), 3.85 (m, 1H), 3.75-3.8 (m, 7H), 3.5 (t, 2), 0.9 (s, 9H). HR-MS FAB m/z for C₂₁H₃₁N₅O₇calcd. 466.2302 (M⁺+1), obsd. 466.2289.

The following compounds were synthesized as described in the above Scheme 5 (Path A), using various resin bound carbamates in the place of 4-2. These compounds were characterized using LC and MS as shown in Table 6.

### EXAMPLE 96

(2S)-2-{[(benzyloxy)carbonyl]amino}-3-({4-[2-(4,5-dihydro-1H-imidazol-2-ylamino)ethoxy]-2-hydroxybenzoyl}amino)propanoic acid.

### EXAMPLE 97

(2S)-3-({4-[2-(4,5-dihydro-1H-imidazol-2-ylamino)ethoxy]-2-hydroxybenzoyl}-amino)-2-[(methoxycarbonyl)amino]propanoic acid.

### EXAMPLE 98

(2S)-3-({4-[2-(4,5-dihydro-1H-imidazol-2-ylamino)ethoxy]-2-hydroxybenzoyl}-amino)-2-[(ethoxycarbonyl)amino]propanoic acid.

### EXAMPLE 99

(2S)-3-({4-[2-(4,5-dihydro-1H-imidazol-2-ylamino)ethoxy]-2-hydroxybenzoyl}-amino)-2-[(propoxycarbonyl)amino]propanoic acid.

### EXAMPLE 100

(2S)-3-({4-[2-(4,5-dihydro-1H-imidazol-2-ylamino)ethoxy]-2-hydroxybenzoyl}-amino)-2-[(isopropoxycarbonyl)amino]propanoic acid.

### EXAMPLE 101

(2S)-2-{[(allyloxy)carbonyl]amino}-3-({4-[2-(4,5-dihydro-1H-imidazol-2-ylamino)-ethoxy]-2-hydroxybenzoyl}amino)propanoic acid.

### EXAMPLE 102

(2S)-2-{[(but-3-enyloxy)carbonyl]amino}-3-({4-[2-(4,5-dihydro-1H-imidazol-2-ylamino)ethoxy]-2-hydroxybenzoyl}amino)propanoic acid.

### EXAMPLE 103

(2S)-3-({4-[2-(4,5-dihydro-1H-imidazol-2-ylamino)ethoxy]-2-hydroxybenzoyl}-amino)-2-{[(prop-2-ynyloxy)carbonyl]amino}propanoic acid.

### EXAMPLE 104

(2S)-3-({4-[2-(4,5-dihydro-1H-imidazol-2-ylamino)ethoxy]-2-hydroxybenzoyl}-amino)-2-{[(hexyloxy)carbonyl]amino}propanoic acid.

### EXAMPLE 105

(2S)-3-({4-[2-(4,5-dihydro-1H-imidazol-2-ylamino)ethoxy]-2-hydroxybenzoyl}-amino)-2-{[(octyloxy)carbonyl]amino}propanoic acid.

### EXAMPLE 106

(2S)-2-[(butoxycarbonyl)amino]-3-({4-[2-(4,5-dihydro-1H-imidazol-2-ylamino)ethoxy]-2-hydroxybenzoyl}amino)propanoic acid.

### EXAMPLE 107

(2S)-3-({4-[2-(4,5-dihydro-1H-imidazol-2-ylamino)ethoxy]-2-hydroxybenzoyl}-amino)-2-[(isobutoxycarbonyl)amino]propanoic acid.

TABLE 6

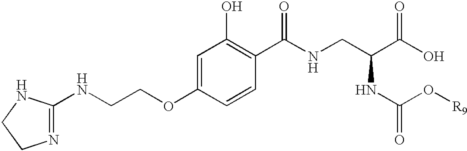

| Ex. | R9 | LC @ 254 nm | (M + H)⁺ | Ex. | R9 | LC @ 254 nm | (M + H)⁺ |
|---|---|---|---|---|---|---|---|
| 97 | Methyl | 2.82 min | 410 | 104 | n-Hexyl | 3.97 min | 480 |
| 98 | Ethyl | 2.99 min | 424 | 105 | n-Octyl | 4.49 min | 508 |
| 99 | n-Propyl | 3.21 min | 438 | 95 | (CH3)3CCH2 | 3.63 min | 466 |
| 100 | i-Propyl | 3.17 min | 438 | 106 | n-Butyl | 3.46 min | 452 |
| 101 | Allyl | 3.13 min | 436 | 107 | i-Butyl | 3.44 min | 452 |
| 102 | Homoallyl | 3.31 min | 450 | 96 | Benzyl | 3.60 min | 486 |
| 103 | Propargyl | 3.01 min | 434 | | | | |

The following compounds were synthesized as described in the above Scheme 5 (Path A), using various resin bound ureas in the place of 4-2. These compounds were characterized using LC and MS as shown in Table 7.

### EXAMPLE 108

(2S)-2-{[(butylamino)carbonyl]amino}-3-({4-[2-(4,5-dihydro-1H-imidazol-2-ylamino)ethoxy]-2-hydroxybenzoyl}amino)propanoic acid.

### EXAMPLE 109

(2S)-3-({4-[2-(4,5-dihydro-1H-imidazol-2-ylamino)ethoxy]-2-hydroxybenzoyl}-amino)-2-{[(hexylamino)carbonyl]amino}propanoic acid.

### EXAMPLE 110

(2S)-3-({4-[2-(4,5-dihydro-1H-imidazol-2-ylamino)ethoxy]-2-hydroxybenzoyl}-amino)-2-{[(octylamino)carbonyl]amino}propanoic acid.

### EXAMPLE 111

(2S)-2-{[(allylamino)carbonyl]amino}-3-({4-[2-(4,5-dihydro-1H-imidazol-2-ylamino)ethoxy]-2-hydroxybenzoyl}amino)propanoic acid.

### EXAMPLE 112

(2S)-2-{[(cyclohexylamino)carbonyl]amino}-3-({4-[2-(4,5-dihydro-1H-imidazol-2-ylamino)ethoxy]-2-hydroxybenzoyl}amino)propanoic acid.

### EXAMPLE 113

(2S)-2-{[(benzylamino)carbonyl]amino}-3-({⁴-[2-(4,5-dihydro-1H-imidazol-2-ylamino)ethoxy]-2-hydroxybenzoyl}amino)propanoic acid.

### EXAMPLE 114

3-({4-[2-(2,5-dihydro-1H-imidazol-4-ylamino)ethoxy]-2-hydroxybenzoyl)amino-N-({{(1S,2R)-phenylcyclopropyl]amino)carbonyl alanine.

### EXAMPLE 115

(2S)-3-({4-[2-(4,5-dihydro-1H-imidazol-2-ylamino)ethoxy]-2-hydroxybenzoyl}-amino)-2-{[(2-methoxyanilino)carbonyl]amino}propanoic acid.

### EXAMPLE 116

(2S)-2-{[([1,1′-biphenyl]-2-ylamino)carbonyl]amino}-3-({4-[2-(4,5-dihydro-1H-imidazol-2-ylamino)ethoxy]-2-hydroxybenzoyl}amino)propanoic acid.

### EXAMPLE 117

(2S)-3-({4-[2-(4,5-dihydro-1H-imidazol-2-ylamino)ethoxy]-2-hydroxybenzoyl}-amino)-2-({[(2-phenylethyl)amino]carbonyl}amino)propanoic acid.

TABLE 7

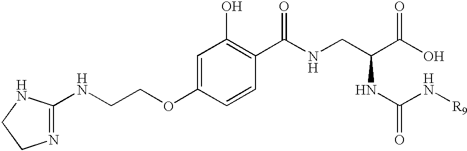

| Ex. | R9 | LC @ 254 nm | (M + H)⁺ | Ex. | R9 | LC @ 254 nm | (M + H)⁺ |
|---|---|---|---|---|---|---|---|
| 108 | 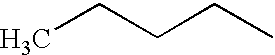 | 3.20 min | 451 | 113 | 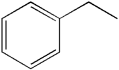 | 3.26 min | 485 |
| 109 | 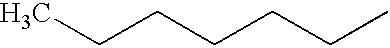 | 3.69 min | 479 | 114 | 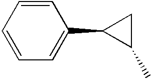 | 3.57 min | 511 |
| 110 | 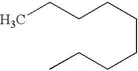 | 4.24 min | 507 | 115 | 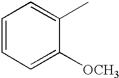 | 3.42 min | 501 |
| 111 | 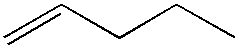 | 2.84 min | 435 | 116 | 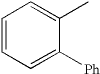 | 3.89 min | 547 |
| 112 | 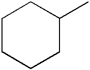 | 3.37 min | 477 | 117 | 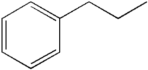 | 3.47 min | 499 |

The following compounds were synthesized as described in the above Scheme 5 (Path A), using various resin bound amides in the place of 4-2. These compounds were characterized using LC and MS as shown in Table 8.

### EXAMPLE 118

(2S)-3-({4-[2-(4,5-dihydro-1H-imidazol-2-ylamino)ethoxy]-2-hydroxybenzoyl}-amino)-2-(isobutyrylamino)propanoic acid.

### EXAMPLE 119

(2S)-2-(butyrylamino)-3-({4-[2-(4,5-dihydro-1H-imidazol-2-ylamino)ethoxy]-2-hydroxybenzoyl}amino)propanoic acid.

### EXAMPLE 120

(2S)-3-({4-[2-(4,5-dihydro-1H-imidazol-2-ylamino)ethoxy]-2-hydroxybenzoyl}-amino)-2-(hexanoylamino)propanoic acid.

### EXAMPLE 121

(2S)-3-({4-[2-(4,5-dihydro-1H-imidazol-2-ylamino)ethoxy]-2-hydroxybenzoyl}-amino)-2-(pentanoylamino)propanoic acid.

### EXAMPLE 122

(2S)-3-({4-[2-(4,5-dihydro-1H-imidazol-2-ylamino)ethoxy]-2-hydroxybenzoyl}-amino)-2-[(3,3-dimethylbutanoyl)amino]propanoic acid.

### EXAMPLE 123

(2S)-3-({4-[2-(4,5-dihydro-1H-imidazol-2-ylamino)ethoxy]-2-hydroxybenzoyl}-amino)-2-{[(2,2,3,3-tetramethylcyclopropyl)carbonyl]amino}propanoic acid.

### EXAMPLE 124

(2S)-2-{[2-(1-adamantyl)acetyl]amino}-3-({4-[2-(4,5-dihydro-1H-imidazol-2-ylamino)ethoxy]-2-hydroxybenzoyl}amino)propanoic acid.

### EXAMPLE 125

(2S)-3-({4-[2-(4,5-dihydro-1H-imidazol-2-ylamino)ethoxy]-2-hydroxybenzoyl}-amino)-2-(pent-4-ynoylamino)propanoic acid.

### EXAMPLE 126

(2S)-2-[(cyclohexylcarbonyl)amino]-3-({4-[2-(4,5-dihydro-1H-imidazol-2-ylamino)ethoxy]-2-hydroxybenzoyl}amino)propanoic acid.

### EXAMPLE 127

(2S)-3-({4-[2-(4,5-dihydro-1H-imidazol-2-ylamino)ethoxy]-2-hydroxybenzoyl}-amino)-2-[(2-phenylacetyl)amino]propanoic acid.

### EXAMPLE 128

(2S)-3-({4-[2-(4,5-dihydro-1H-imidazol-2-ylamino)ethoxy]-2-hydroxybenzoyl}-amino)-2-[(3-phenylpropanoyl)amino]propanoic acid.

### EXAMPLE 129

(2S)-2-[(2-cyclohexylacetyl)amino]-3-({4-[2-(4,5-dihydro-1H-imidazol-2-ylamino)ethoxy]-2-hydroxybenzoyl}amino)propanoic acid.

### EXAMPLE 130

(2S)-3-({4-[2-(4,5-dihydro-1H-imidazol-2-ylamino)ethoxy]-2-hydroxybenzoyl}-amino)-2-{[(E)-3-phenylprop-2-enoyl]amino}propanoic acid.

### EXAMPLE 131

(2S)-2-[(2-chlorobenzoyl)amino]-3-({4-[2-(4,5-dihydro-1H-imidazol-2-ylamino)ethoxy]-2-hydroxybenzoyl}amino)propanoic acid.

### EXAMPLE 132

(2S)-3-({4-[2-(4,5-dihydro-1H-imidazol-2-ylamino)ethoxy]-2-hydroxybenzoyl}amino)-2-[(2-methylbenzoyl)amino]propanoic acid.

### EXAMPLE 133

(2S)-3-({4-[2-(4,5-dihydro-1H-imidazol-2-ylamino)ethoxy]-2-hydroxybenzoyl}-amino)-2-[(2-methoxybenzoyl)amino]propanoic acid.

### EXAMPLE 134

(2S)-2-[(4-chlorobenzoyl)amino]-3-({4-[2-(4,5-dihydro-1H-imidazol-2-ylamino) -ethoxy]-2-hydroxybenzoyl}amino)propanoic acid.

### EXAMPLE 135

(2S)-3-({4-[2-(4,5-dihydro-1H-imidazol-2-ylamino)ethoxy]-2-hydroxybenzoyl}-amino)-2-[(4-methylbenzoyl)amino]propanoic acid.

### EXAMPLE 136

(2S)-3-({4-[2-(4,5-dihydro-1H-imidazol-2-ylamino)ethoxy]-2-hydroxybenzoyl}-amino)-2-[(4-methoxybenzoyl)amino]propanoic acid.

### EXAMPLE 137

(2S)-3-({4-[2-(4,5-dihydro-1H-imidazol-2-ylamino)ethoxy]-2-hydroxybenzoyl}-amino)-2-[(2,5-dimethyl-3-furoyl)amino]propanoic acid.

### EXAMPLE 138

(2S)-2-[(2-bromobenzoyl)amino]-3-({4-[2-(4,5-dihydro-1H-imidazol-2-ylamino-ethoxy]-2-hydroxybenzoyl}amino)propanoic acid.

### EXAMPLE 139

(2S)-2-[(4-bromobenzoyl)amino]-3-({4-[2-(4,5-dihydro-1H-imidazol-2-ylamino)ethoxy]-2-hydroxybenzoyl}amino)propanoic acid.

### EXAMPLE 140

(2S)-3-({4-[2-(4,5-dihydro-1H-imidazol-2-ylamino)ethoxy]-2-hydroxybenzoyl}-amino)-2-[(2,3-dimethylbenzoyl)amino]propanoic acid.

### EXAMPLE 141

(2S)-2-[(3-chlorobenzoyl)amino]-3-({4-[2-(4,5-dihydro-1H-imidazol-2-ylamino)-ethoxy]-2-hydroxybenzoyl}amino)propanoic acid.

TABLE 8

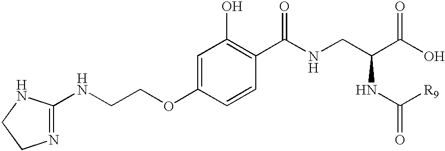

| Ex. | R9 | LC @ 254 nm | (M + H)⁺ | Ex. | R9 | LC @ 254 nm | (M + H)⁺ |
|---|---|---|---|---|---|---|---|
| 118 | 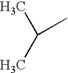 | 2.90 min | 422 (M + H) | 130 | 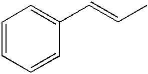 | 3.57 min | 482 (M + H) |
| 119 | 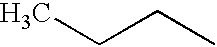 | 2.90 min | 422 (M + H) | 131 | 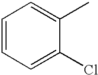 | 3.24 min | 490 (M + H) |
| 120 | 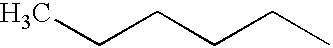 | 3.34 min | 450 (M + H) | 132 | 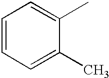 | 3.30 min | 470 (M + H) |
| 121 | 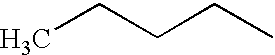 | 3.10 min | 436 (M + H) | 133 | 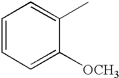 | 3.38 min | 486 (M + H) |
| 122 | 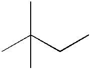 | 3.26 min | 450 (M + H) | 134 | 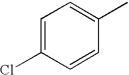 | 3.59 min | 490 (M + H) |
| 123 | 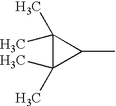 | 3.71 min | 476 (M + H) | 135 | 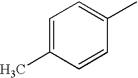 | 3.46 min | 470 (M + H) |
| 124 | 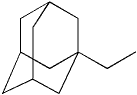 | 3.90 min | 528 (M + H) | 136 | 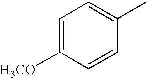 | 3.34 min | 486 (M + H) |
| 125 | 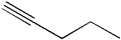 | 2.86 min | 432 (M + H) | 137 | 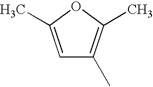 | 3.45 min | 474 (M + H) |
| 126 | 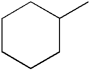 | 3.36 min | 462 (M + H) | 138 | 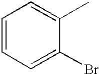 | 3.16 min | 534 (M + H) |
| 127 | 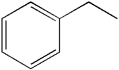 | 3.22 min | 470 (M + H) | 139 | 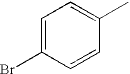 | 3.28 min | 534 (M + H) |
| 128 | 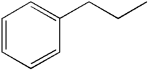 | 3.42 min | 484 (M + H) | 140 | 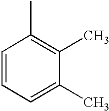 | 3.65 min | 484 (M + H) |
| 129 | 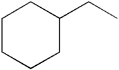 | 3.50 min | 476 (M + H) | 141 | 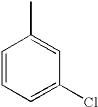 | 3.56 min | 490 (M + H) |

### EXAMPLE 142

### (2S)-3-({2-hydroxy-4-[2-(3,4,5,6-tetrahydro-2H-azepin-7-ylamino)ethoxy]benzoyl}amino)-2-{[(neopentyloxy)carbonyl]amino}propanoic acid (5-4)

### (2S)-3-({2-hydroxy-4-[2-(3,4,5,6-tetrahydro-2H-azepin-7-ylamino)ethoxy]benzoyl}-amino)-2-{[(neopentyloxy)carbonyl]amino}propanoic acid on Wang Resin (5-3)

The resin 4-2 (100 mg: 0.1 mmole) was swollen in dioxane and treated with a solution of 1-aza-2-methoxy-1-cycloheptene (127 mg; 1 mmole) in dioxane (1.5 mL). The reaction mixture was shaken at room temperature for 18 h. The mixture was filtered and the resin was washed with dioxane (4×4 mL), methanol (4×mL) and dichloromethane (4×4 mL). The resin was dried under vacuum. A sample of the resin was removed and subjected to Kaiser Ninhydrin test. If the test showed the presence of free amine (resin turned blue) the coupling described above was repeated.

The resin 5-3 was cleaved by tratment with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. This crude product 5-4 was purified via preparative HPLC. NMR (300 MHz, DMSO-d6) δ 12.8 (s, 1H), 9.55 (t, 1H), 9.25 (t, 1H), 8.8 (t, 1H), 7.8 (d, J=9 Hz, 1H), 7.7 (d, J=8 Hz, 1H), 7.3 (m, 5H), 6.5 (m, 2H), 5.0 (s, 2H), 4.3 (q, 1H), 4.2 (t, 2H), 3.8 (m, 3H), 3.6 (m, 1H), 3.5 (m, 2H), 2.7 (m, 2H), 1.7 (m, 2H), 1.6 (m, 4H).

The following compounds were synthesized as described in the above Scheme 5 (Path B), using various resin bound carbamates in the place of 4-2. These compounds were characterized using LC and MS as shown in Table 9.

### EXAMPLE 143

(2S)-2-{[(benzyloxy)carbonyl]amino}-3-({2-hydroxy-4-[2-(3,4,5,6-tetrahydro-2-azepin-7-ylamino)ethoxy]benzoyl}amino)propanoic acid.

### EXAMPLE 144

(2S)-3-({2-hydroxy-4-[2-(3,4,5,6-tetrahydro-2H-azepin-7-ylamino)ethoxy]benzoyl}-amino)-2-[(methoxycarbonyl)amino]propanoic acid.

### EXAMPLE 145

(2S)-2-[(ethoxycarbonyl)amino]-3-({2-hydroxy-4-[2-(3,4,5,6-tetrahydro-2H-azepin-7-ylamino)ethoxy]benzoyl}amino)propanoic acid.

### EXAMPLE 146

(2S)-3-({2-hydroxy-4-[2-(3,4,5,6-tetrahydro-2H-azepin-7-ylamino)ethoxy]benzoyl}amino)-2-[(propoxycarbonyl)amino]propanoic acid.

### EXAMPLE 147

(2S)-3-({2-hydroxy-4-[2-(3,4,5,6-tetrahydro-2H-azepin-7-ylamino)ethoxy]benzoyl}-amino)-2-[(isopropoxycarbonyl)amino]propanoic acid.

### EXAMPLE 148

(2S)-2-{[(allyloxy)carbonyl]amino}-3-({2-hydroxy-4-[2-(3,4,5,6-tetrahydro-2H-azepin-7-ylamino)ethoxy]benzoyl}amino)propanoic acid.

### EXAMPLE 149

(2S)-2-{[(but-3-enyloxy)carbonyl]amino}-3-({2-hydroxy-4-[2-(3,4,5,6-tetrahydro-2H-azepin-7-ylamino)ethoxy]benzoyl}amino)propanoic acid.

### EXAMPLE 150

(2S)-3-({2-hydroxy-4-[2-(3,4,5,6-tetrahydro-2H-azepin-7-ylamino)ethoxy]benzoyl }-amino)-2-{[(prop-2-ynyloxy)carbonyl]amino}propanoic acid.

### EXAMPLE 151

(2S)-2-{[(hexyloxy)carbonyl]amino}-3-({2-hydroxy-4-[2-(3,4,5,6-tetrahydro-2H-azepin-7-ylamino)ethoxy]benzoyl}amino)propanoic acid.

### EXAMPLE 152

(2S)-3-({2-hydroxy-4-[2-(3,4,5,6-tetrahydro-2H-azepin-7-ylamino)ethoxy]benzoyl}-amino)-2-{[(octyloxy)carbonyl]amino}propanoic acid.

### EXAMPLE 153

(2S)-3-({2-hydroxy-4-[2-(3,4,5,6-tetrahydro-2H-azepin-7-ylamino)ethoxy]benzoyl}-amino)-2-{[(2,2,2-trichloroethoxy)carbonyl]amino}propanoic acid.

### EXAMPLE 154

(2S)-2-[(butoxycarbonyl)amino]-3-({2-hydroxy-4-[2-(3,4,5,6-tetrahydro-2H-azepin-7-ylamino)ethoxy]benzoyl}amino)propanoic acid.

### EXAMPLE 155

(2S)-3-({2-hydroxy-4-[2-(3,4,5,6-tetrahydro-2H-azepin-7-ylamino)ethoxy]-yl}amino)-2-[(isobutoxycarbonyl)amino]propanoic acid.

TABLE 9

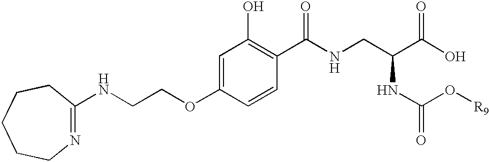

| Ex. | R9 | LC @ 254 nm | (M + H)⁺ | Ex. | R9 | LC @ 254 nm | (M + H)⁺ |
|---|---|---|---|---|---|---|---|
| 144 | Methyl | 3.08 min | 437 | 151 | n-Hexyl | 3.19 min | 507 |
| 145 | Ethyl | 3.25 min | 451 | 152 | n-Octyl | 4.67 min | 535 |
| 146 | n-Propyl | 3.46 min | 465 | 142 | (CH3)3CH2 | 3.85 min | 493 |
| 147 | i-Propyl | 3.38 min | 465 | 153 | (CCl3)3CCH2 | 3.89 min | 553 |
| 148 | Allyl | 3.37 min | 463 | 154 | n-Butyl | 3.70 min | 479 |
| 149 | Homoallyl | 3.55 min | 477 | 155 | i-Butyl | 3.67 min | 479 |
| 150 | Propargyl | 3.27 min | 461 | 143 | Benzyl | 3.83 min | 513 |

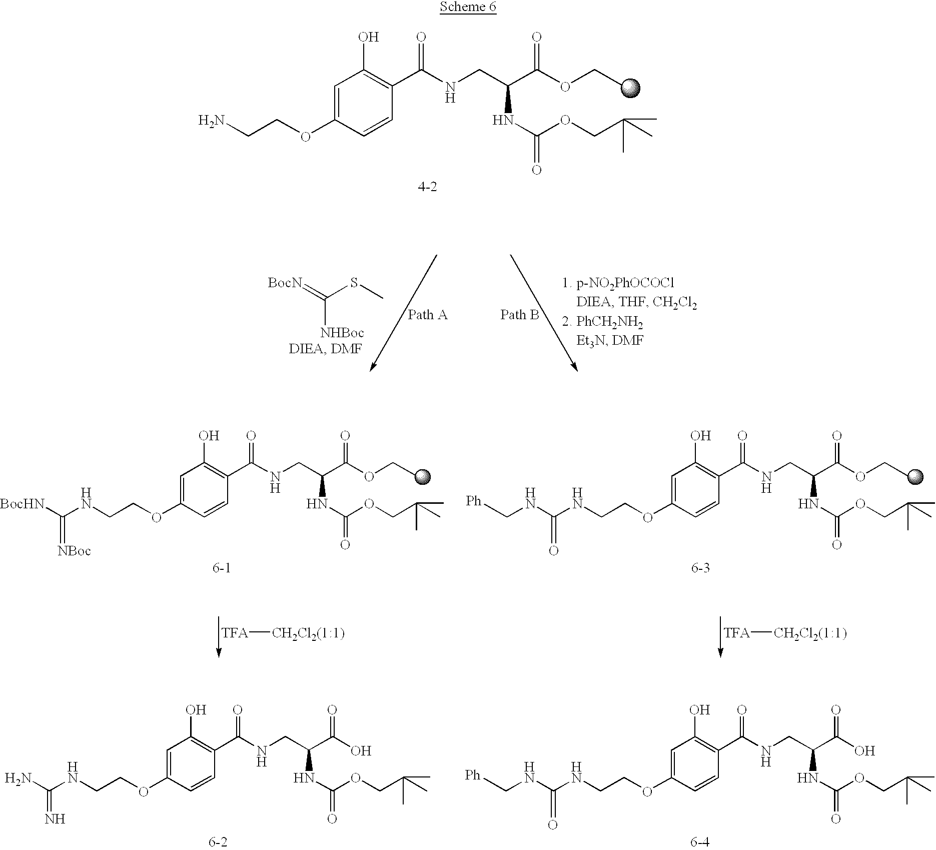

### EXAMPLE 156

### (2S)-3-{[4-(2-{[amino(imino)methyl]amino}ethoxy)-2-hydroxy-benzoyl]amino}-2-{[(neopentyloxy)carbonyl]amino}propanoic acid (6-2)

### (2S)-3-{[4-(2-{[amino(imino)methyl]amino}ethoxy)-2-hydroxybenzoyl]amino}-2-{[(neopentyloxy)carbonyl]amino}propanoic acid on Wane Resin (6-1)

The resin 4-2 (100 mg; 0.1 mmole) was swollen in DMF and treated with a solution of 1,3-bis(tert-butoxycarbonyl)-2-methyl-2-thiopseudourea (145 mg; (0.5 mmole) in DMF (1.5 mL) followed by diisopropylamine (0.15 mL; 1 mmole). The reaction mixture was shaken at room temperature for 18 h. The mixture was filtered and the resin was washed with dimethylformamide (4×4 mL), methanol (4×mL) and dichloromethane (4×4mL). The resin was dried under vacuum. A sample of the resin was removed and subjected to Kaiser Ninhydrin test. If the test showed the presence of free amine (resin turned blue) the coupling described above was repeated.

The resin 6-1 was cleaved by tratment with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. This crude product 6-2 was purified via preparative HPLC. NMR (300 MHz, MeOH-d4) δ 7.7 (d, J=7 Hz, 1H), 6.5 (m, 2H), 4.5 (q, 1H), 4.2 (m, 2H), 3.85 (m, 1H), 3.8 (m, 2H), 3.75 (m, 1H), 3.7 (m, 2H), 0.9 (s, 9H).

HR-MS FAB m/z for C₁₉H₂₉N₅O₇calcd. 440.2145 (M⁺+1), obsd. 440.2154.

The following compounds were synthesized as described in the above Scheme 6 (Path A), using various resin bound carbamates in the place of 4-2. These compounds were characterized using LC and MS as shown in Table 10.

### EXAMPLE 157

(2S)-3-{[4-(2-{[amino(imino)methyl]amino}ethoxy)-2-hydroxybenzoyl]amino}-2-{[(benzyloxy)carbonyl]amino}propanoic acid.

### EXAMPLE 158

(2S)-3-{[4-(2-{[amino(imino)methyl]amino)ethoxy)-2-hydroxybenzoyl]amino}-2-[(methoxycarbonyl)amino]propanoic acid.

### EXAMPLE 159

(2S)-3-{[4-(2-{[amino(imino)methyl]amino)ethoxy)-2-hydroxybenzoyl]amino}-2-[(ethoxycarbonyl)amino]propanoic acid.

### EXAMPLE 160

(2S)-3-{[4-(2-{[amino(imino)methyl]amino}ethoxy)-2-hydroxybenzoyl]amino}-2-[(propoxycarbonyl)amino]propanoic acid.

### EXAMPLE 161

(2S)-3-{[4-(2-{[amino(imino)methyl]amino}ethoxy)-2-hydroxybenzoyl]amino}-2-[(isopropoxycarbonyl)amino]propanoic acid.

### EXAMPLE 162

(2S)-2-{[(allyloxy)carbonyl]amino}-3-{[4-(2-{[amino(imino)methyl]amino}ethoxy-2-hydroxybenzoyl]amino}propanoic acid.

### EXAMPLE 163

(2S)-3-{[4-(2-{[amino(imino)methyl]amino}ethoxy)-2-hydroxybenzoyl]amino}-2-{[(but-3-enyloxy)carbonyl]amino}propanoic acid.

### EXAMPLE 164

(2S)-3-{[4-(2-{[amino(imino)methyl]amino}ethoxy)-2-hydroxybenzoyl]amino}-2-[(butoxycarbonyl)amino]propanoic acid.

### EXAMPLE 165

(2S)-3-{[4-(2-{[amino(imino)methyl]amino}ethoxy)-2-hydroxybenzoyl]amino}-2-{[(2,2,2-trichloroethoxy)carbonyl]amino}propanoic acid.

### EXAMPLE 166

(2S)-3-{[4-(2-{[amino(imino)methyl]amino}ethoxy)-2-hydroxybenzoyl]amino}-2-{[(hexyloxy)carbonyl]amino}propanoic acid.

### EXAMPLE 167

(2S)-3-{[4-(2-{[amino(imino)methyl]amino}ethoxy)-2-hydroxybenzoyl]amino}-2-{[(prop-2-ynyloxy)carbonyl]amino}propanoic acid.

### EXAMPLE 168

(2S)-3-{[4-(2-{[amino(imino)methyl]amino}ethoxy)-2-hydroxybenzoyl]amino}-2-{[([1,1′-biphenyl]-2-ylmethoxy)carbonyl]amino}propanoic acid.

### EXAMPLE 169

(2S)-3-{[4-(2-{[amino(imino)methyl]amino}ethoxy)-2-hydroxybenzoyl]amino}-2-({[(4-bromobenzyl)oxy]carbonyl}amino)propanoic acid.

### EXAMPLE 170

(2S)-3-{[4-(2-{[amino(imino)methyl]amino}ethoxy)-2-hydroxybenzoyl]amino}-2-({[(4-fluorobenzyl)oxy]carbonyl}amino)propanoic acid.

### EXAMPLE 171

(2S)-3-{[4-(2-{[amino(imino)methyl]amino}ethoxy)-2-hydroxybenzoyl]amino}-2-({[(2-bromobenzyl)oxy]carbonyl}amino)propanoic acid.

### EXAMPLE 172

(2S)-3-{[4-(2-{[amino(imino)methyl]amino}ethoxy)-2-hydroxybenzoyl]amino}-2-[({[4-(trifluoromethyl)benzyl]oxy}carbonyl)amino]propanoic acid.

TABLE 10

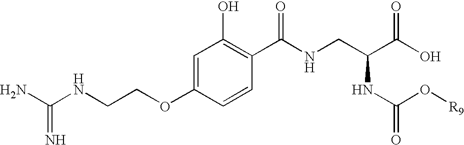

| Ex. | R9 | LC @ 254 nm | (M + H)⁺ | Ex. | R9 | LC @ 254 nm | (M + H)⁺ |
|---|---|---|---|---|---|---|---|
| 158 | Methyl | 2.75 min | 384 | 165 | (CCl3)3CCH2 | 3.60 min | 502 |
| 159 | Ethyl | 2.93 min | 397 | 164 | n-Butyl | 3.39 min | 426 |
| 160 | n-Propyl | 3.15 min | 412 | 157 | Benzyl | 3.53 min | 460 |
| 161 | i-Propyl | 3.11 min | 412 | 168 | 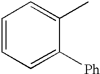 | 4.20 min | 536 |
| 162 | Allyl | 3.05 min | 410 | 169 | 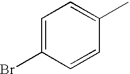 | 3.85 min | 539 |
| 163 | Homoallyl | 3.25 min | 424 | 170 | 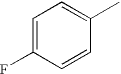 | 3.60 min | 478 |
| 167 | Propargyl | 2.95 min | 408 | 171 | 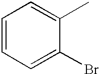 | 3.81 min | 539 |
| 166 | n-Hexyl | 3.91 min | 4454 | 172 | 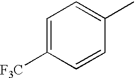 | 3.97 min | 528 |
| 156 | (CH3)3CCH2 | 3.57 min | 440 | | | | |

The following compounds were synthesized as described in the above Scheme 6 (Path A), using various resin bound ureas in the place of 4-2. These compounds were characterized using LC and MS as shown in Table 11.

### EXAMPLE 173

(2S)-3-{[4-(2-{[amino(imino)methyl]amino}ethoxy)-2-hydroxybenzoyl]amino}-2-[(2-toluidinocarbonyl)amino]propanoic acid.

### EXAMPLE 174

(2S)-3-{[4-(2-{[amino(imino)methyl]amino}ethoxy)-2-hydroxybenzoyl]amino}-2-{[(2-methoxyanilino)carbonyl]amino}propanoic acid.

### EXAMPLE 175

(2S)-3-{[4-(2-{[amino(imino)methyl]amino}ethoxy)-2-hydroxybenzoyl]amino}-2-{[(2-chloroanilino)carbonyl]amino}propanoic acid.

### EXAMPLE 176

(2S)-3-{[4-(2-{[amino(imino)methyl]amino}ethoxy)-2-hydroxybenzoyl]amino}-2-{[(2-bromoanilino)carbonyl]amino}propanoic acid.

### EXAMPLE 177

(2S)-3-{[4-(2-{[amino(imino)methyl]amino}ethoxy)-2-hydroxybenzoyl]amino}-2-[([1,1′-biphenyl]-2-ylamino)carbonyl]amino}propanoic acid.

### EXAMPLE 178

(2S)-3-{[4-(2-{[amino(imino)methyl]amino}ethoxy)-2-hydroxybenzoyl]amino}-2-[(4-toluidinocarbonyl)amino]propanoic acid.

### EXAMPLE 179

(2S)-3-{[4-(2-{[amino(imino)methyl]amino}ethoxy)-2-hydroxybenzoyl]amino}-2-({[4-(trifluoromethoxy)anilino]carbonyl}amino)propanoic acid.

### EXAMPLE 180

(2S)-3-{[4-(2-{[amino(imino)methyl]amino}ethoxy)-2-hydroxybenzoyl]amino}-2-{[(4-chloroanilino)carbonyl]amino}propanoic acid.

### EXAMPLE 181

(2S)-3-{[4-(2-{[amino(imino)methyl]amino}ethoxy)-2-hydroxybenzoyl]amino}-2-{[(4-fluoroanilino)carbonyl]amino}propanoic acid

### EXAMPLE 182

(2S)-2-{[(4-acetylanilino)carbonyl]amino}-3-{[4-(2-{[amino(imino)methyl]-amino}ethoxy)-2-hydroxybenzoyl]amino}propanoic acid.

### EXAMPLE 183

(2S)-3-{[4-(2-{[amino(imino)methyl]amino}ethoxy)-2-hydroxybenzoyl]amino}-2-{[(cyclohexylamino)carbonyl]amino}propanoic acid.

### EXAMPLE 184

(2S)-3-{[4-(2-{[amino(imino)methyl]amino}ethoxy)-2-hydroxybenzoyl]amino}-2-{[(1-naphthylamino)carbonyl]amino}propanoic acid.

### EXAMPLE 185

(2S)-3-{[4-(2-{[amino(imino)methyl]amino}ethoxy)-2-hydroxybenzoyl]amino}-2-{[(benzylamino)carbonyl]amino}propanoic acid.

### EXAMPLE 186

(2S)-3-{[4-(2-{[amino(imino)methyl]amino}ethoxy)-2-hydroxybenzoyl]amino}-2-([(2-phenylethyl)amino]carbonyl}amino)propanoic acid.

### EXAMPLE 187

(2S)-3-{[4-(2-{[amino(imino)methyl]amino}ethoxy)-2-hydroxybenzoyl]amino}-2-{[(octylamino)carbonyl]amino}propanoic acid.

### EXAMPLE 188

(2S)-3-{[4-(2-{[amino(imino)methyl]amino}ethoxy)-2-hydroxybenzoyl]amino}-2-{[(4-methoxyanilino)carbonyl]amino)}propanoic acid.

### EXAMPLE 189

(2S)-3-{[4-(2-{[amino(imino)methyl]amino}ethoxy)-2-hydroxybenzoyl]amino}-2-[(anilinocarbonyl)amino]propanoic acid.

TABLE 11

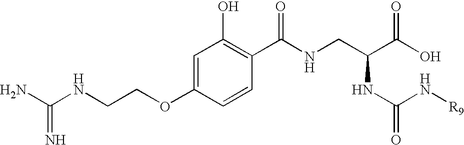

| Ex. | R1 | LC @ 254 nm | (M + H)⁺ | Ex. | R1 | LC @ 254 nm | (M + H)⁺ |
|---|---|---|---|---|---|---|---|
| 187 | 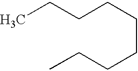 | 4.18 min | 481 | 182 | 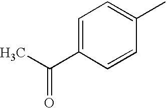 | 3.26 min | 487 |
| 189 | 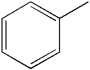 | 3.29 min | 445 | 175 | 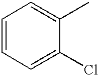 | 3.46 min | 481 |
| 183 | 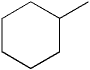 | 3.32 min | 451 | 176 | 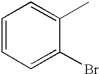 | 3.48 min | 525 |
| 185 | 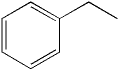 | 3.20 min | 459 | 177 | 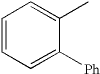 | 3.81 min | 521 |
| 173 | 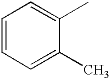 | 3.32 min | 459 | 181 | 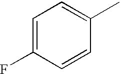 | 3.39 min | 463 |
| 178 | 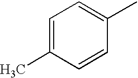 | 3.50 min | 459 | 180 | 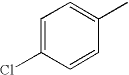 | 3.68 min | 481 |
| 188 | 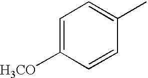 | 3.27 min | 475 | 184 | 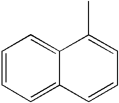 | 3.57 min | 495 |
| 174 | 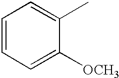 | 3.36 min | 475 | 186 | 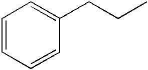 | 3.37 min | 473 |
| | | | | 179 | 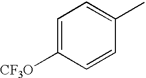 | 3.92 min | 529 |

The following compounds were synthesized as described in the above Scheme 6 (Path A), using various resin bound amides in the place of 4-2. These compounds were characterized using LC and MS as shown in Table 12.

### EXAMPLE 190

(2S)-3-{[4-(2-{[amino(imino)methyl]amino}ethoxy)-2-hydroxybenzoyl]amino}-2-(isobutyrylamino)propanoic acid.

### EXAMPLE 191

(2S)-3-{[4-(2-{[amino(imino)methyl]amino}ethoxy)-2-hydroxybenzoyl]amino}-2-(butyrylamino)propanoic acid.

### EXAMPLE 192

(2S)-3-{[4-(2-{[amino(imino)methyl]amino}ethoxy)-2-hydroxybenzoyl]amino}-2-(hexanoylamino)propanoic acid.

### EXAMPLE 193

(2S)-3-{[4-(2-{[amino(imino)methyl]amino}ethoxy)-2-hydroxybenzoyl]amino}-2-(pentanoylamino)propanoic acid.

### EXAMPLE 194

(2S)-3-{[4-(2-{[amino(imino)methyl]amino}ethoxy)-2-hydroxybenzoyl]amino}-2-[(3,3-dimethylbutanoyl)propanoic acid.

### EXAMPLE 195

(2S)-3-{[4-(2-{[amino(imino)methyl]amino}ethoxy)-2-hydroxybenzoyl]amino}-2-{[(2,2,3,3-tetramethylcyclopropyl)carbonyl]amino}propanoic acid.

### EXAMPLE 196

(2S)-2-{[2-(1-adamantyl)acetyl]amino}-3-{[4-(2-{[amino(imino)methyl]amino}-ethoxy)-2-hydroxybenzoyl]amino}propanoic acid.

### EXAMPLE 197

(2S)-3-{[4-(2-{[amino(imino)methyl]amino}ethoxy)-2-hydroxybenzoyl]amino}-2-(pent-4-ynoylamino)propanoic acid.

### EXAMPLE 198

(2S)-3-{[4-(2-{[amino(imino)methyl]amino}ethoxy)-2-hydroxybenzoyl]amino}-2-[(cyclohexylcarbonyl)amino]propanoic acid.

### EXAMPLE 199

(2S)-3-{[4-(2-{[amino(imino)methyl]amino}ethoxy)-2-hydroxybenzoyl]amino}-2-[(2-phenylacetyl)amino]propanoic acid.

### EXAMPLE 200

(2S)-3-{[4-(2-{[amino(imino)methyl]amino}ethoxy)-2-hydroxybenzoyl]amino}-2-[(3-phenylpropanoyl)amino]propanoic acid.

### EXAMPLE 201

(2S)-3-{[4-(2-{[amino(imino)methyl]amino}ethoxy)-2-hydroxybenzoyl]amino}-2-[(2-cyclohexylacetyl)amino]propanoic acid.

### EXAMPLE 202

(2S)-3-{[4-(2-{[amino(imino)methyl]amino}ethoxy)-2-hydroxybenzoyl]amino}-2-{[(E)-3-phenylprop-2-enoyl]amino}propanoic acid.

### EXAMPLE 203

(2S)-3-{[4-(2-{[amino(imino)methyl]amino}ethoxy)-2-hydroxybenzoyl]amino}-2-[(2-chlorobenzoyl)amino]propanoic acid.

### EXAMPLE 204

(2S)-3-{[4-(2-{[amino(imino)methyl]amino}ethoxy)-2-hydroxybenzoyl]amino}-2-[(2-methylbenzoyl)amino]propanoic acid.

### EXAMPLE 205

(2S)-3-{[4-(2-{[amino(imino)methyl]amino}ethoxy)-2-hydroxybenzoyl]amino}-2-[(2-methoxybenzoyl)amino]propanoic acid.

### EXAMPLE 206

(2S)-3-{[4-(2-{[amino(imino)methyl]amino}ethoxy)-2-hydroxybenzoyl]amino}-2-[(4-chlorobenzoyl)amino]propanoic acid.

### EXAMPLE 207

(2S)-3-{[4-(2-{[amino(imino)methyl]amino}ethoxy)-2-hydroxybenzoyl]amino}-2-[(4-methylbenzoyl)amino]propanoic acid.

### EXAMPLE 208

(2S)-3-{[4-(2-{[amino(imino)methyl]amino}ethoxy)-2-hydroxybenzoyl]amino-}-2-[(4-methoxybenzoyl)amino]propanoic acid.

### EXAMPLE 209

(2S)-3-{[4-(2-{[amino(imino)methyl]amino}ethoxy)-2-hydroxybenzoyl]amino}-2-[(pyridin-3-ylcarbonyl)amino]propanoic acid.

### EXAMPLE 210

(2S)-3-{[4-(2-{[amino(imino)methyl]amino}ethoxy)-2-hydroxybenzoyl]amino}-2-(isonicotinoylamino)propanoic acid.

### EXAMPLE 211

(2S)-3-{[4-(2-{[amino(imino)methyl]amino}ethoxy)-2-hydroxybenzoyl]amino}-2-[(2,5-dimethyl-3-furoyl)amino]propanoic acid.

### EXAMPLE 212

(2S)-3-{[4-(2-{[amino(imino)methyl]amino}ethoxy)-2-hydroxybenzoyl]amino-}-2-[(2-bromobenzoyl)amino]propanoic acid.

### EXAMPLE 213

(2S)-3-{[4-(2-{[amino(imino)methyl]amino}ethoxy)-2-hydroxybenzoyl]amino}-2-[(4-bromobenzoyl)amino]propanoic acid.

### EXAMPLE 214

(2S)-3-{[4-(2-{[amino(imino)methyl]amino}ethoxy)-2-hydroxybenzoyl]amino}-2-[(2,3-dimethylbenzoyl)amino]propanoic acid.

### EXAMPLE 215

(2S)-3-{[4-(2-{[amino(imino)methyl]amino}ethoxy)-2-hydroxybenzoyl]amino}-2-[(3-chlorobenzoyl)amino]propanoic acid.

### EXAMPLE 216

(2S)-3-{[4-(2-{[amino(imino)methyl]amino}ethoxy)-2-hydroxybenzoyl]amino}-2-(benzoylamino)propanoic acid.

### EXAMPLE 217

(2S)-3-{[4-(2-{[amino(imino)methyl]amino}ethoxy)-2-hydroxybenzoyl]amino}-2-[(4-ethylbenzoyl)amino]propanoic acid.

### EXAMPLE 218

(2S)-3-{[4-(2-{[amino(imino)methyl]amino}ethoxy)-2-hydroxybenzoyl]amino}-2-[(4-butoxybenzoyl)amino]propanoic acid.

TABLE 12

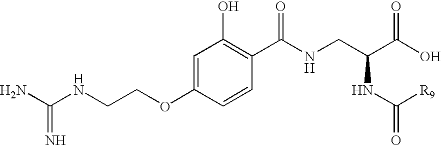

| Ex. | R9 | LC @ 254 nm | (M + H)⁺ |
|---|---|---|---|
| 190 | 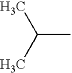 | 2.88 min | 396(M + H) |
| 191 | 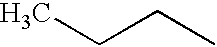 | 2.88 min | 396(M + H) |
| 192 | 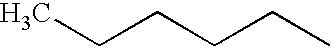 | 3.34 min | 424(M + H) |
| 193 | 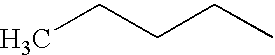 | 3.09 min | 410(M + H) |
| 194 | 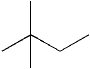 | 3.24 min | 424(M + H) |
| 195 | 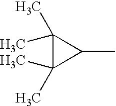 | 3.70 min | 450(M + H) |
| 196 | 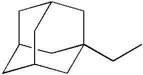 | 3.85 min | 502(M + H) |
| 197 | 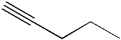 | 2.84 min | 406(M + H) |
| 198 | 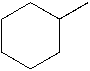 | 3.35 min | 436(M + H) |
| 199 | 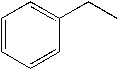 | 3.21 min | 444(M + H) |
| 200 | 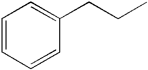 | 3.42 min | 458(M + H) |
| 201 | 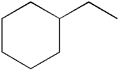 | 3.50 min | 450(M + H) |
| 202 | 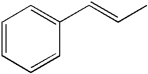 | 3.55 min | 456(M + H) |
| 203 | 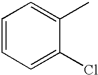 | 3.25 min | 464(M + H) |
| 204 | 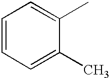 | 3.29 min | 444(M + H) |
| 205 | 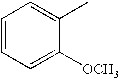 | 3.38 min | 460(M + H) |
| 206 | 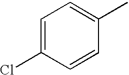 | 3.58 min | 464(M + H) |
| 207 | 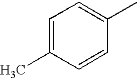 | 3.45 min | 444(M + H) |
| 208 | 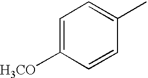 | 3.33 min | 460(M + H) |
| 209 | 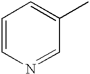 | 2.61 min | 431(M + H) |
| 210 | 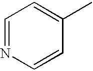 | 2.58 min | 431(M + H) |
| 211 | 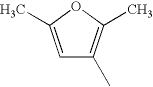 | 3.45 min | 448(M + H) |
| 212 | 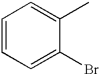 | 3.27 min | 509(M + H) |
| 213 | 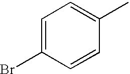 | 3.65 min | 509(M + H) |
| 214 | 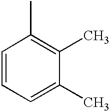 | 3.65 min | 458(M + H) |
| 215 | 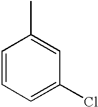 | 3.46 min | 464(M + H) |
| 216 | 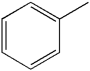 | 3.19 min | 430(M + H) |
| 217 | 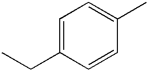 | 3.66 min | 458(M + H) |
| 218 | 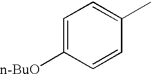 | 4.08 min | 502(M + H) |

### EXAMPLE 219

### (2S)-3-{[4-(2-{[(benzylamino)carbonyl]amino}ethoxy)-2-hydroxy-benzoyl]amino}-2-{[(neopentyloxy)carbonyl]amino}propanoic acid (6-4)

### (2S)-3-{[4-(2-{[benzylamino)carbonyl]amino}ethoxy)-2-hydroxybenzoyl]amino}-2-{[(neopentyloxy)carbonyl]amino}propanoic acid on Wang Resin (6-3)

The resin 4-2 (100 mg; 0.1 mmole) was swollen in 1:1 tetrahydrofuran and dichloromethane. To it was added a solution of 4-nitrophenylchloroformate (50 mg; 0.25 mmole) in 1:1 THF:DCM (1.5 mL) followed by diisopropylamine (0.075 mL; 0.5 mmole). The reaction mixture was shaken at room temperature for 30 min. The mixture was filtered and the resin was washed with THF (4×4 mL) and dichloromethane (4×4 mL) and dried. The resin was suspended in DMF (1.5 mL) and benzyl amine (54 mg; 0.5 mmole) was added followed by triethylamine (101 mg; 1 mmole). The reaction mixture was shaken at room temperature for 2 h. The mixture were filtered and the resin in each vessel was washed with dimethylformamide (4×4 mL), methanol (4×mL) and dichloromethane (4×4 mL). The resin was dried under vacuum.

The resin 6-3 was cleaved by treatment with dichloromethane (0.5 mL) and trifluroacetic acid (0.5 mL) for 30 min at room temperature. The reaction mixture was filtered and the resin was washed with dichloromethane. The filtrate was concentrated and dried in vacuo on a Savant Speed Vac Plus. This crude product 6-4 was purified via preparative HPLC. NMR (300 MHz, MeOH-d4) δ 7.65 (d, J=7 Hz, 1H), 7.25 (m, 5H), 6.5 (m, 2H), 4.4 (q, 1H), 4.3 (s, 2H), 4.15 (t, 2H), 3.85 (m, 1H), 3.75 (m, 3H), 3.5 (t, 2H). 0.9 (s, 9H).

HR-MS FAB m/z for C₂₆H₃₄N₄O₈calcd. 531.2455 (M⁺+1), obsd. 531.2459.

The following compounds were synthesized as described in the above Scheme 6 (Path B), using various amines in the place of benzyl amine. These compounds were characterized using LC and MS as shown in Table 13.

### EXAMPLE 220

(2S)-3-{[4-(2-{[(benzylamino)carbonyl]amino}ethoxy)-2-hydroxybenzoyl]amino}-2-{[(benzyloxy)carbonyl]amino}propanoic acid.

### EXAMPLE 221

(2S)-3-{[4-(2-{[(benzylamino)carbonyl]amino}ethoxy)-2-hydroxybenzoyl]amino}-2-[(methoxycarbonyl)amino]propanoic acid.

### EXAMPLE 222

(2S)-3-{[4-(2-{[(benzylamino)carbonyl]amino}ethoxy)-2-hydroxybenzoyl]amino}-2-[(ethoxycarbonyl)amino]propanoic acid.

### EXAMPLE 223

(2S)-3-{[4-(2-{[(benzylamino)carbonyl]amino}ethoxy)-2-hydroxybenzoyl]amino}-2-[(propoxycarbonyl)amino]propanoic acid.

### EXAMPLE 224

(2S)-3-{[4-(2-{[(benzylamino)carbonyl]amino}ethoxy)-2-hydroxybenzoyl]amino}-2-[(isopropoxycarbonyl)amino]propanoic acid.

### EXAMPLE 225

(2S)-2-{[(allyloxy)carbonyl]amino}-3-{[4-(2-{[(benzylamino)carbonyl]amino}-ethoxy)-2-hydroxybenzoyl]amino}propanoic acid.

### EXAMPLE 226

(2S)-3-{[4-(2-{[(benzylamino)carbonyl]amino}ethoxy)-2-hydroxybenzoyl]amino}-2-{[(but-3-enyloxy)carbonyl]amino}propanoic acid.

### EXAMPLE 227

(2S)-3-{[4-(2-{[(benzylamino)carbonyl]amino}ethoxy)-2-hydroxybenzoyl]amino}-2-{[(prop-2-ynyloxy)carbonyl]amino}propanoic acid.

### EXAMPLE 228

(2S)-3-{[4-(2-{[(benzylamino)carbonyl]amino}ethoxy)-2-hydroxybenzoyl]amino}-2-{[(hexyloxy)carbonyl]amino}propanoic acid.

### EXAMPLE 229

(2S)-3-{[4-(2-{[(benzylamino)carbonyl]amino}ethoxy)-2-hydroxybenzoyl]amino}-2-{[(octyloxy)carbonyl]amino}propanoic acid.

### EXAMPLE 230

(2S)-3-{[4-(2-{[(benzylamino)carbonyl]amino}ethoxy)-2-hydroxybenzoyl]amino}-2-{[(2,2,2-trichloroethoxy)carbonyl]amino}propanoic acid.

### EXAMPLE 231

(2S)-3-{[4-(2-{[(benzylamino)carbonyl]amino}ethoxy)-2-hydroxybenzoyl]amino}-2-[(butoxycarbonyl)amino]propanoic acid.

### EXAMPLE 232

(2S)-3-{[4-(2-{[(benzylamino)carbonyl]amino}ethoxy)-2-hydroxybenzoyl]amino}-2-[(isobutoxycarbonyl)amino]propanoic acid.

### EXAMPLE 233

(2S)-2-{[(benzyloxy)carbonyl]amino}-3-({2-hydroxy-4-[2-({[(pyridin-3-ylmethyl-amino]carbonyl}amino)ethoxy]benzoyl}amino)propanoic acid.

### EXAMPLE 234

(2S)-3-({2-hydroxy-4-[2-({[(pyridin-3-ylmethyl)amino]carbonyl}-amino)ethoxy]-benzoyl}amino)-2-[(methoxycarbonyl)amino]propanoic acid.

### EXAMPLE 235

(2S)-2-[(ethoxycarbonyl)amino]-3-({2-hydroxy-4-[2-({[(pyridin-3-ylmethyl)amino]-carbonyl}amino)ethoxy]benzoyl}amino)propanoic acid.

### EXAMPLE 236

(2S)-3-({2-hydroxy-4-[1-({(pyridin-3-ylmethyl)amino]carbonyl}amino)ethoxy]-benzoyl}amino)-2-[(propoxycarbonyl)amino]propanoic acid.

### EXAMPLE 237

(2S)-3-({2-hydroxy-4-[2-({[(pyridin-3-ylmethyl)amino]carbonyl}-amino)ethoxy]-benzoyl}amino)-2-[(isopropoxycarbonyl)amino]propanoic acid.

### EXAMPLE 238

(2S)-2-{[(allyloxy)carbonyl]amino}-3-({2-hydroxy-4-[2-({[(pyridin-3-ylmethyl)amino]carbonyl}amino)ethoxy]benzoyl}amino)propanoic acid.

### EXAMPLE 239

(2S)-2-{[(but-3-enyloxy)carbonyl]amino}-3-({2-hydroxy-4-[2-({[(pyridin-3-ylmethyl)amino]carbonyl}amino)ethoxy]benzoyl}amino)propanoic acid

### EXAMPLE 240

(2S)-3-({2-hydroxy-4-[2-({[(pyridin-3-ylmethyl)amino]carbonyl}-amino)ethoxy]-benzoyl}amino)-2-{[(prop-2-ynyloxy)carbonyl]amino}propanoic acid.

### EXAMPLE 241

(2S)-2-{[(hexyloxy)carbonyl]amino}-3-({2-hydroxy-4-[2-({[(pyridin-3-ylmethyl)-amino]carbonyl}amino)ethoxy]benzoyl}amino)propanoic acid.

### EXAMPLE 242

(2S)-3-({2-hydroxy-4-[2-({[(pyridin-3-ylmethyl)amino]carbonyl}-amino)ethoxy]-benzoyl}amino)-2-{[(octyloxy)carbonyl]amino}propanoic acid.

### EXAMPLE 243

(2S)-3-({2-hydroxy-4-[2-({[(pyridin-3-ylmethyl)amino]carbonyl}-amino)ethoxy]-benzoyl}amino)-2-{[(neopentyloxy)carbonyl]amino}propanoic acid.

### EXAMPLE 244

(2S)-3-({2-hydroxy-4-[2-({[(pyridin-3-ylmethyl)amino]carbonyl}amino)-ethoxy]benzoyl}amino)-2-{[(2,2,2-trichloroethoxy)carbonyl]amino}propanoic acid.

### EXAMPLE 245

(2S)-2-[(butoxycarbonyl)amino]-3-({2-hydroxy-4-[2-({[(pyridin-3-ylmethyl)amino]carbonyl}amino)ethoxy]benzoyl}amino)propanoic acid.

### EXAMPLE 246

(2S)-3-({2-hydroxy-4-[2-({[(pyridin-3-ylmethyl)amino]carbonyl}-amino)ethoxy]-benzoyl}amino)-2-[(isobutoxycarbonyl)amino]propanoic acid.

### EXAMPLE 247

(2S)-2-{[(benzyloxy)carbonyl]amino}-3-({2-hydroxy-4-[2-({[(pyridin-4-ylmethyl)amino]carbonyl}amino)ethoxy]benzoyl}amino)propanoic acid.

### EXAMPLE 248

(2S)-3-({2-hydroxy-4-[2-({[(pyridin-4-ylmethyl)amino]carbonyl}-amino)-ethoxy]benzoyl}amino)-2-[(methoxycarbonyl)amino]propanoic acid.

### EXAMPLE 249

(2S)-2-[(ethoxycarbonyl)amino]-3-({2-hydroxy-4-[2-({[(pyridin-4-ylmethyl)-amino]carbonyl}amino)ethoxy]benzoyl}amino)propanoic acid.

### EXAMPLE 250

(2S)-3-({2-hydroxy-4-[2-({[(pyridin-4-ylmethyl)amino]carbonyl}-amino)ethoxy]benzoyl}amino)-2-[(propoxycarbonyl)amino]propanoic acid.

### EXAMPLE 251

(2S)-3-({2-hydroxy-4-[2-({[(pyridin-4-ylmethyl)amino]carbonyl}-amino)ethoxy]benzoyl}amino)-2-[(isopropoxycarbonyl)amino]propanoic acid.

### EXAMPLE 252

(2S)-2-{[(allyloxy)carbonyl]amino}-3-({2-hydroxy-4-[2-({[(pyrydin-4-ylmethyl)amino]carbonyl}amino)ethoxy]benzoyl}amino)propanoic acid.

### EXAMPLE 253

(2S)-2-{[(but-3-enyloxy)carbonyl]amino}-3-({2-hydroxy-4-[2-({[(pyridin-4-ylmethyl)amino]carbonyl}amino)ethoxy]benzoyl}amino)propanoic acid.

### EXAMPLE 254

(2S)-3-({2-hydroxy-4-[2-({[(pyridin-4-ylmethyl)amino]carbonyl}-amino)ethoxy]-benzoyl}amino)-2-{[(prop-2-ynyloxy)carbonyl]amino}propanoic acid.

### EXAMPLE 255

(2S)-2-{[(hexyloxy)carbonyl]amino}-3-({2-hydroxy-4-[2-({[(pyridin-4-ylmethyl)amino]carbonyl}amino)ethoxy]benzoyl}amino)propanoic acid.

### EXAMPLE 256

(2S)-3-({2-hydroxy-4-[2-({[(pyridin-4-ylmethyl)amino]carbonyl}-amino)ethoxy]-benzoyl}amino)-2-{[(octyloxy)carbonyl]amino}propanoic acid.

### EXAMPLE 257

(2S)-3-({2-hydroxy-4-[2-({[(pyridin-4-ylmethyl)amino]carbonyl}-amino)ethoxy]-benzoyl}amino)-2-{[(neopentyloxy)carbonyl]amino}propanoic acid.

### EXAMPLE 258

(2S)-3-({2-hydroxy-4-[2-({[(pyridin-4-ylmethyl)amino]carbonyl}amino)-ethoxy]-benzoyl}amino)-2-{[(2,2,2-trichloroethoxy)carbonyl]amino}propanoic acid.

### EXAMPLE 259

(2S)-2-[(butoxycarbonyl)amino]-3-({2-hydroxy-4-[2-({[(pyridin-4-ylmethyl)amino]-carbonyl}amino)ethoxy]benzoyl}amino)propanoic acid.

### EXAMPLE 260

(2S)-3-({2-hydroxy-4-[2-({[(pyridin-4-ylmethyl)amino]carbonyl}-amino)ethoxy]-benzoyl}amino)-2-[(isobutoxycarbonyl)amino]propanoic acid.

### EXAMPLE 261

(2S)-2-{[(benzyloxy)carbonyl]amino}-3-({2-hydroxy-4-[2-({[(4-methylbenzyl)-amino]carbonyl}amino)ethoxy]benzoyl}amino)propanoic acid. LC 4.75 min., M+H 565.

### EXAMPLE 262

(2S)-2-{[(benzyloxy)carbonyl]amino}-3-({2-hydroxy-4-[2-({[(4-methoxybenzyl)-amino]carbonyl}amino)ethoxy]benzoyl}amino)propanoic acid. LC 3.75 min., M+H 581.

### EXAMPLE 263

(2S)-2-{[(benzyloxy)carbonyl]amino}-3-({4-[2-({[(4-chlorobenzyl)amino]-carbonyl}-amino)ethoxy]-2-hydroxybenzoyl}amino)propanoic acid. LC 4.83 min., M+H 5.86.

### EXAMPLE 264

(2S)-2-{[(benzyloxy)carbonyl]amino}-3-[(4-{2-[({[4-(dimethylamino)benzyl]-amino}carbonyl)amino]ethoxy}-2-hydroxybenzoyl)amino]propanoic acid. LC 3.7 min., M+H 594.

### EXAMPLE 265

(2S)-3-[(4-{2-[({[4-(aminosulfonyl)benzyl]amino}carbonyl)amino]ethoxy}-2-hydroxybenzoyl)amino]-2-{[(benzyloxy)carbonyl]amino}propanoic acid. LC 4.08 min., M+H 630.

### EXAMPLE 266

(2S)-2-{[(benzyloxy)carbonyl]amino}-3-[(2-hydroxy-4-{2-[({[4-(trifluoromethoxy)-benzyl]amino}carbonyl)amino]ethoxy}benzoyl)amino]propanoic acid. LC 5.06 min., M+H 635.

### EXAMPLE 267

(2S)-2-{[(benzyloxy)carbonyl]amino}-3-({4-[2-({[(2-chlorobenzyl)amino]carbonyl}-amino)ethoxy]-2-hydroxybenzoyl}amino)propanoic acid. LC 4.8 min., M+H 586.

### EXAMPLE 268

(2S)-2-{[(benzyloxy)carbonyl]amino}-3-({2-hydroxy-4-[2-({[(2-methylbenzyl)amino[-carbonyl}amino)ethoxy]benzoyl}amino)propanoic acid. LC 4.74 min., M+H 565.

### EXAMPLE 269

(2S)-2-{[(benzyloxy)carbonyl]amino}-3-({4-[2-({[(2-bromobenzyl)amino]-carbonyl}-amino)ethoxy]-2-hydroxybenzoyl}amino)propanoic acid. LC 4.85 min., M+H 630.

### EXAMPLE 270

(2S)-2-{[(benzyloxy)carbonyl]amino}-3-({4-[2-({[(2,4-dichlorobenzyl)amino]-carbonyl}amino)ethoxy]-2-hydroxybenzoyl}amino)propanoic acid. LC 5.08 min., M+H 620.

### EXAMPLE 271

(2S)-3-({4-[2-({[(2-aminobenzyl)amino]carbonyl}amino)ethoxy]-2-hydroxybenzoyl}-amino)-2-{[(benzyloxy)carbonyl]amino}propanoic acid. LC 3.81 min., M+H 566.

### EXAMPLE 272

(2S)-2-{[(benzyloxy)carbonyl]amino}-3-({2-hydroxy-4-[2-({[(pyridin-2-ylmethyl)-amino]carbonyl}amino)ethoxy]benzoyl}amino)propanoic acid. LC 3.58 min., M+H 552.

TABLE 13

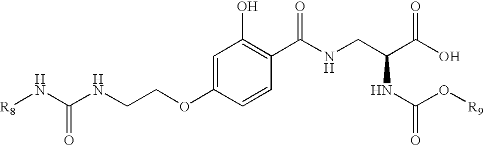

| Ex. | 221-227 | 248-254 | 234-240 |
|---|---|---|---|
| 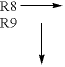 | 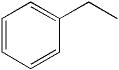 | 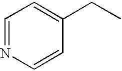 | 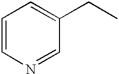 |
| methyl | 475 M + H | 476 M + H | 476 M + H |
| | 3.84 min | 2.84 min | 2.84 min |
| | 221 | 248 | 234 |
| ethyl | 489 M + H | 490 M + H | 490 M + H |
| | 4.00 min | 3.01 min | 2.99 min |
| | 222 | 249 | 235 |
| n-propyl | 503 M + H | 504 M + H | 504 M + H |
| | 4.21 min | 3.20 min | 3.19 min |
| | 223 | 250 | 236 |
| i-propyl | 503 M + H | 504 M + H | 504 M + H |
| | 4.19 min | 3.17 min | 3.17 min |
| | 224 | 251 | 237 |
| allyl | 501 M + H | 502 M + H | 502 M + H |
| | 4.14 min | 3.12 min | 3.12 min |
| | 225 | 252 | 238 |
| homo- | 515 M + H | 516 M + H | 516 M + H |
| allyl | 4.31 min | 3.28 min | 3.29 min |
| | 226 | 253 | 239 |
| propargyl | 499 M + H | 500 M + H | 500 M + H |
| | 4.06 min | 3.01 min | 3.02 min |
| | 227 | 254 | 240 |
| | 219-220 | 247 & | 233 & |
| | 228-232 | 255-260 | 241-246 |
| 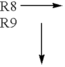 | 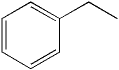 | 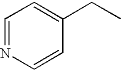 | 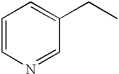 |
| | 219-220 & | | |
| | 228-232 | | |
| hexyl | 545 M + H | 546 M + H | 546 M + H |
| | 4.90 min | 3.90 min | 3.90 min |
| | 228 | 255 | 241 |
| octyl | 573 M + H | 574 M + H | 574 M + H |
| | 5.37 min | 4.38 min | 4.37 min |
| | 229 | 256 | 242 |
| (CH3)3CCH2— | 531 M + H | 532 M + H | 532 M + H |
| | 4.58 min | 3.57 min | 3.58 min |
| | 219 | 257 | 243 |
| (CCl3)3CCH2— | 593 M + H | 594 M + H | 594 M + H |
| | 4.62 min | 3.62 min | 3.62 min |
| | 230 | 258 | 244 |
| n-butyl | 517 M + H | 518 M + H | 518 M + H |
| | 4.43 min | 3.43 min | 3.43 min |
| | 231 | 259 | 245 |
| i-butyl | 517 M + H | 518 M + H | 518 M + H |
| | 4.41 min | 3.40 min | 3.40 min |
| | 232 | 260 | 246 |
| benzyl | 551 M + H | 552 M + H | 552 M + H |
| | 4.59 min | 3.52 min | 3.55 min |
| | 220 | 247 | 233 |

Alternatively, Schemes 7-12 demonstrate the solution phase synthesis practice of this invention with detailed synthetic procedures for representative compounds.

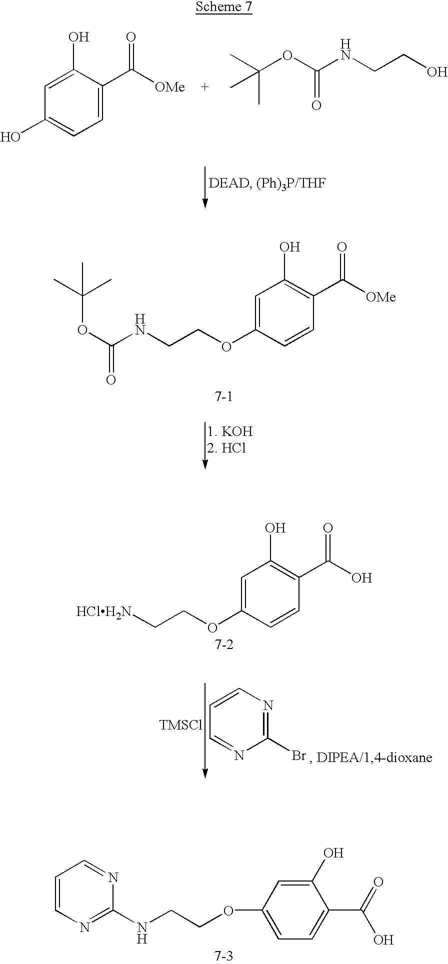

### EXAMPLE 273

### 2(S)-Benzenesulfonylamino-3-[2-hydroxy-4-(2-pyrimidin-2-ylamino)-ethoxy]benzoylamino]propionic acid ethyl ester (8-1)

### Methyl 4-[2-N-(t-butoxycarbonyl)ethoxyl]-2-hydroxy benzoate (7-1)

Methyl 2,4-dihydroxy benzoate (14.5 g, Aldrich), 2-(N-t-butoxycarbonyl)ethanol (13.9 g, Aldrich) and triphenyl phosphine (22.6 g, Aldrich) were combined in 350 mL of THF and cooled in ice under N₂ atmosphere. Diethyl diazodicarboxylate (DEAD, 15 g, Aldrich) was added, the ice bath removed and the reaction mixture allowed to stir at ambient temperature for 15 h. The solvent was removed on a rotary evaporator and the residue chromatographed on silica gel (300 g, Merck silica 60), elution with CH₂Cl₂ to give 18 g of methyl 4-[2-N-(t-butoxycarbonyl)ethoxy]-2-hydroxy benzoate, as a viscous oil. NMR (300 MHz, CDCl₃) δ 11.0 (s, 1 H), 9.5 (d, J=8 Hz, 1H), 6.4 (m, 2H), 5.0 (broad, 1H), 4.0 (t, J=5 Hz, 2H), 3.91 (s, 3H), 3.54 (m, 2H), 1.45 (s, 9H), MS (+ESI) m/z 334 (M+Na)⁺.

### 4-(2-Aminoethoxy)-2-hydroxybenzoic acid, hydrochloride (7-2)

Ester 7-1 (7.2 g) was treated with 5eq. KOH (dissolved in minimum amount of water and equal volume of 1,4-dioxane) at room temperature until TLC indicated complete absence of starting material (3-12 h). The reaction mixture was acidified (pH=6) with the addition of 1N HCl solution and extracted with ethyl acetate. The extract was washed with saturated aqueous brine solution, dried over MgSO₄, filtered and concentrated on the rotary evaporator. The crude product (5.34 g) was recrystallized from ether, then dissolved in 1,4-dioxane and treated with an excess of anhydrous HCl (4M in dioxane, Aldrich). The mixture was allowed to stand at ambient temperature for 24 h. Volatile materials were removed in vacuo on the rotary evaporator to give 7-2 as a hydroscopic off-white solid. NMR (400 MHz, DMSO-d6) δ 13.6 (broad, 1H), 11.6 (broad, 1H), 8.3 (broad, 3H), 7.7 (d, J=9 Hz, 2H), 6.53 (m, 2H), 4.23 (t, J=5 Hz, 2H), 3.2 (s, broad, 2H).

### 2-Hydroxy-4-[2-(pyrimidine-2ylamino)ethoxy]benzoic acid (7-3)

A mixture of compound 7-2 (20 g), diissopropylethylamine (DIPEA, 74 mL), trimethylsilylchloride (TMSCl, 21.6 mL) and 2-bromopyrimidine (Lancaster, 13.5 g) were combined in 350 mL 1,4-dioxane at room temperature, then brought to reflux under N₂ atmosphere. After 2 days, an additional 12 mL trimethylsilyl chloride was added, and the mixture continued at reflux for an additional 2 days (until TLC showed no stating material remained). The reaction mixture was cooled to ambient temperature, concentrated to dryness in vacuo on a rotary evaporator and the residue suspended in water. The heterogeneous mixture was refluxed briefly, allowed to cool to room temperature, the product collected on a vacuum filter and air dried to give 15.3 g of 7-3, as a tan powder. NMR (400 MHz, DMSO-d₆) δ 12 (very broad, 2H) 8.3 (d, J=5 Hz, 2H) 7.7 (d, J=9 Hz, 1H), 7.28 (t, J=6 Hz, 1H), 6.57 (t, J=5 Hz, 1H), 6.49 (m, 2H), 4.13 (t, J=6 Hz, 2H), 3.62 (q, 2H); MS (+ESI) m/z 276 (M+H)⁺; IR (KBr) ν (cm⁻¹) 3275, 3000, 1660, 1625.

A mixture of compound 7-3 (5.51 g), N-hydroxybenzotriazole hydrate(HOBt.H₂O, 4.6 g, Aldrich), N-methyl morpholine (NMM,8.8 mL) and 1-[3-(dimethylamino)-propyl]-3-ethyl carbodimide hydrochloride (DEC, 7.6 g, Aldrich) were stirred at room temperature in 60 mL DMF for ˜0.3 h, followed by the addition of 2(S)-benzenelsulfonylamino-β-alanine ethyl ester (WO9532710, Scheme 2). The mixture was allowed to stir at room temperature for 2 days under N₂ atmosphere. Volatile materials were removed in vacuo on a rotary evaporator, and the residue dissolved in ethanol. Twenty grams of silica gel (silica 60, Merck) were added and the solvent removed. Chromatography on 300 g of silica gel (ethyl acetate elution, gave 8.1 g of the title compound as a pale yellow glass, which upon hardening and pulverizing produced an off-white powder. NMR (400 MHz, DMSO-d₆) δ 12.5 (s, 1H), 8.68 (t, J=6 Hz, 1H), 8.48 (d, J=9 Hz, 1H), 8.27 (d, J=5 Hz, 1H), 7.73 (m, 2H), 7.64 (d, J=9 Hz, 1H), 7.55-7.5 (m, 1H), 7.48-7.44 (m, 2H), 7.27 (t, J=6 Hz, 1H), 6.58 (t, J=5 Hz, 1H), 6.47 (dd, J=6 Hz, 3 Hz, 1H), 6.42 (d, J=2 Hz, 1H), 4.1 (t, J=6 Hz, 2H), 4.05 (q, J=7 Hz, 1H), 3.79 (q, J=7 Hz), 3.62 (q, 2H), 3.54 (m, 1H), 3.34 (m, 1H overlapping with H₂O peak), 0.94 (t, J=7 Hz); MS (+ESI) m/z 530 (M+H)⁺; IR (KBr) ν (cm⁻¹) 3400, 1740, 1650, 1580.

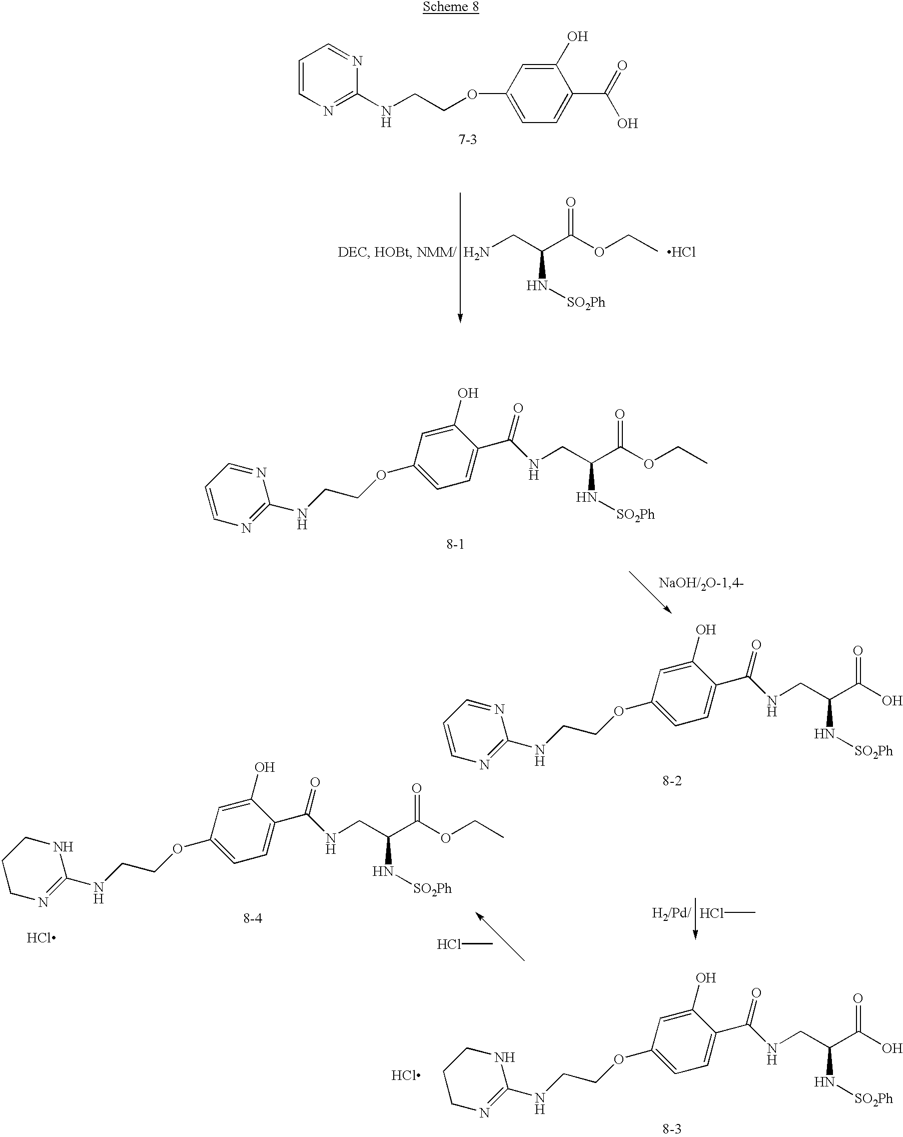

### EXAMPLE 274

### 2(S)-Benzenesulfonylamino-3-[2-hydroxy-4-(2-pyrimidin-2-ylamino)-ethoxy]benzoylamino]propionic acid (8-2).

To a solution of compound 8-1 (8.1 g), dissolved in 75 mL 1,4-dioxane, was added a solution of NaOH (4 g) in 75 mL H₂O and the reaction mixture was stirred at room temperature for 15 h. The mixture was concentrated in vacuo and the residue partitioned between water and dichloromethane. The aqueous phase was acidified with 1N aqueous HCl solution to pH 7, which produced a gum precipitate. This material (7 g) was absorbed onto 15 g of silica gel as in example 1, followed by chromatography on 200 g silica gel. Elution with chloroform (90)-methanol (10)-acetic acid (0.1) gave the title compound as an amber syrup. MS (−ESI) m/z 500 (M−H); [α]_{D}²⁵=6.84 (c. 9.497, methanol).

Analysis for: C₂₂H₂₃N₅O₇S:

| | |
|---|---|
| Calculated: | C, 52.69; H, 4.62; N, 13.96. |
| Found: | C, 52.65; H, 4.43; N, 13.6. |

### EXAMPLE 275

### 2(S)-Benzenesulfonylamino-3-[2-hydroxy-4-[2-(3,4,5,6-tetrahydro-pyrimidin-2-ylamino)ethoxy]benzoylamino]propionic acid hydrochloride (8-3).

A mixture of compound 8-2 (8 g) and 10% Pd/C (1 g) was stirred at room temperature in 1,4-dioxane (125 mL), acetic acid (125 mL), water (50 mL) and concentrated HCl (2 mL) under H₂ atmosphere (balloon) for 2 days. Celite was added to the mixture with stirring for 0.25 h, and the mixture was filtered through a pad of celite with the aid of isopropanol. The filtrate was concentrated on the rotary evaporator and the residue treated sequentially with (1)warm heptane, then concentrated; (2) 1:1 water -1,4-dioxane, then filtered and concentrated, followed by vacuum drying in an abderhalden apparatus (isopropanol, reflux) to give the title compound 8-3 (4.7 g) as a hygroscopic white powder. NMR (400 MHz, DMSO-d₆) δ 12.6 (broad, 2H) 8.77 (broad, 1H), 8.2 (broad, 1H), 8.1 (broad, 2H), 7.72 (m, 3H), 7.47-7.37 (m, 3H), 6.46 (m, 1H), 6.44 (s, broad, 1H), 4.07 (t, J=5 Hz, 2H), 3.93 (broad, 1H), 3.63-3.43 (m, 1H), 3.4-3.25 (m, 2H), 1.9-1.77 (m, 2H); IR (KBr) ν (cm⁻¹) 3360, 1720, 1580; MS (+ESI) m/z 506 (M+H)⁺.

### EXAMPLE 276

### 2(S)-Benzenesulfonylamino-3-[2-hydroxy-4-(2-pyrimidin-2-ylamino)-ethoxy]benzoylamino]propionic acid ethyl ester hydrochloride (8-4)

The above acid (8-3) was dissolved in ethanol (25 mL) and concentrated HCl (1 mL). The mixture was heated to reflux for 15 h, concentrated on a rotary evaporator and filtered through a short plug of silica gel with the aid of ethanol to give the title compound as a hygroscopic tan powder. IR(KBr) ν (cm⁻¹) 1745, 1690; MS (+ESI) m/z 534 (M+H)⁺.

Analysis for C₂₄H₃₁N₅O₇S.HCl.

| | |
|---|---|
| Calculated: | C, 50.57; H, 5.66; N, 12.29. |
| Found: | C, 50.71; H, 5.66; N, 12.53 |

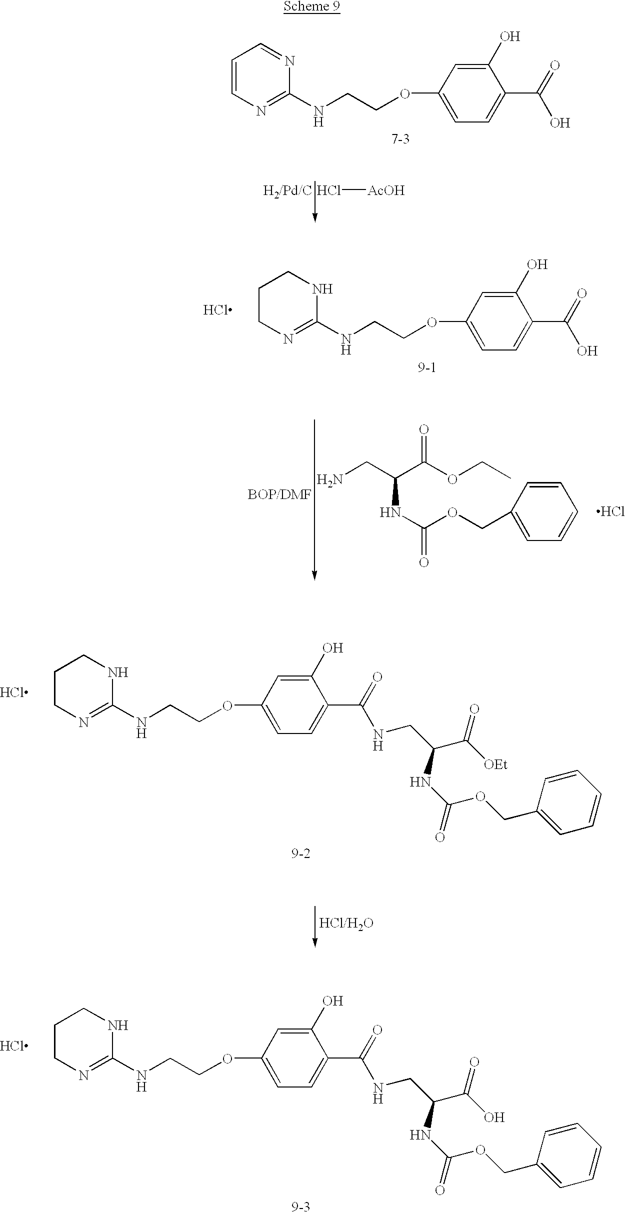

### EXAMPLE 277

### 2(S)-Benzyloxycarbonylamino-3-[2-hydroxy-4-[2-(3,4,5,6-tetra-hydropyrimidin-2-ylamino)ethoxy]benzoylamino]propionic acid ethyl ester hydrochloride (9-2).

### 2-Hydroxy-4-[2-(3,4,5,6-tetrahydropyrimidin-2ylamino)ethoxy]-benzoic acid (9-1).

Compound 7-3 (2 g) was combined with 10% Pd/C (0.5 g), acetic acid (100 mL) and concentrated hydrochloric acid (0.7 mL). The mixture was stirred at room temperature under an atmosphere of H₂ (balloon) for 2 days. Celite was added and the mixture stirred for 0.5 h, then filtered through a pad of celite with the aid of isopropanol. Volatile materials were removed on the rotary evaporator and the residue warmed with heptane (˜0.5 h, 100° C.) followed by concentration in vacuo to give 9-1 as a tan foam. NMR (400 MHz, DMSO-d₆) δ 12.9 (broad, 2H), 8.25 (s, broad, 2H), 7.85 (t, J=6 Hz, 1H), 7.66 (d, J=9 Hz, 1H), 6.48-6.41 (m, 2H), 4.07 (t, J=5 Hz, 2H), 3.56-3.50 (m, 2H), 3.22 (m, 2H, overlapping with H₂O peak), 1.79 (m, 2H); IR (KBr) ν (cm⁻¹) 3450 (broad); MS (+ESI) m/z 280 (M+H)⁺.

Compound 9-1 (1.58 g), 3-amino-2(S)-benzyloxycarbonylaminopropionic acid, ethyl ester hydrochloride (1.51 g; from the amino acid (Fluka) esterified with HCl in ethanol), N-methyl morpholine (NMM, 1.52 g) and benzotriazol-1-yloxytris(dimethylamino) phosphoniumhexafluorophosphate (BOP, 2.21 g) were combined in 40 mL anhydrous DMF. The mixture was stirred for 48 h at room temperature, additional BOP reagent (1 g) was added and the reaction stirred for 15 h. Volatile materials were removed on the rotary evaporator, the residue dissolved in ethanol and absorbed onto 20 g silica gel, and this added to the top of a 200 g silica gel column. Flash chromatography, elution with chloroform-methanol-acetic acid (90:10:1) followed by treatment with an equivalent concentrated aqueous HCl in ethanol and concentration provided the title compound as a hygroscopic tan powder. NMR (400 MHz, DMSO-d₆) δ 12.7 (s, 1H), 8.85 (t, J=6 Hz, 1H), 8.00 (s, broad, 2H), 7.84 (d, J=7.5 Hz, 1H), 7.80 (d, J=9 Hz, 1H), 7.60 (t, J=6 Hz, 1H), 7.32 (m, 5H), 6.49-6.45 (m, 2H), 5.02 (s, 2H), 4.27 (q, 1H), 4.06 (m, 4H), 3.66-3.55 (m, 2H), 3.49 (m, 2H), 3.23 (m, 4H), 1.79 (m, 2H), 1.10 (t, J=7 Hz, 3H); IR (KBr) ν (cm^{31 1}) 3300, 1730, 1650; MS (+ESI) m/z 528 (M+H)⁺.

### EXAMPLE 278

### 2(S)-Benzyloxycarbonylamino-3-[2-hydroxy-4-[2-(3,4,5,6-tetrahydro-pyrimidin-2-ylamino)ethoxy]benzoylamino]propionic acid hydrochloride (9-3).

The above ester 9-2 was hydrolyzed to the title compound 9-3 by refluxing (15-24 h) 1N aqueous HCl solution. When TLC indicated no starting ester remained, the solution was concentrated on the rotary evaporator and the residue treated with warm isopropanol, filtered and concentrated to give 9-3 as a hygroscopic tan powder. MS (+FAB) m/z 500 (M+H)⁺; [α]_{D}²⁵=−7.83 (c. 5.36, MeOH).

Analysis for C₂₄H₂₉N₅O₇.HCl.H₂O;

| | |
|---|---|
| Calculated: | C, 52.03; H, 5.82; N, 12.64. |
| Found: | C, 52.02, H, 5.53; N, 12.00. |

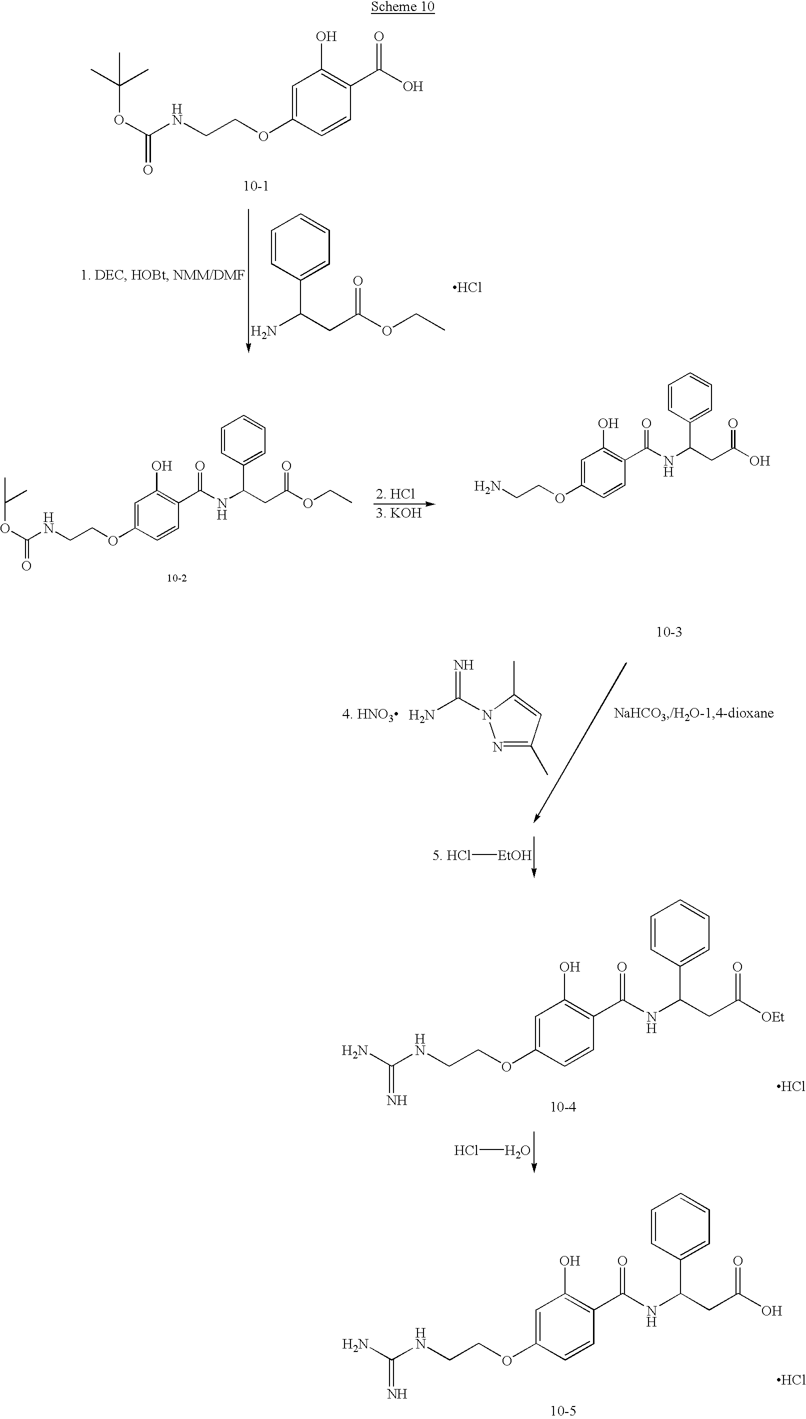

### EXAMPLE 279

### 3-[4-(2-Guanidinoethoxy)-2-hydroxy-benzoylamino]-3-phenylpropanoic acid ethyl ester hydrochloride. (10-4)

Compound 10-1 (1.33 g, scheme 7) and β-phenylalanine ethyl ester hydrochloride (1.03 g; from ethanol—HCl treatment of β-phenylalanine (Aldrich)) were combined in dichloromethane (20 mL) with DEC coupling agent (0.94 g), HOBt (0.75 g) and NMM (0.99 g). The mixture was stirred at room temperature for 15 h. Volatile materials were removed in vacuo on a rotary evaporator and the residue partitioned between ethyl ether and IN aqueous HCl solution. The organic phase was washed with saturated aqueous brine solution, dried over MgSO₄, filtered and concentrated to give 2.8 g of crude coupling product 10-2 (Scheme 10). The N-terminal Boc group was removed by dissolving the product in a minimum amount of absolute ethanol and adding an equal volume of anhydrous HCl in 1,4-dioxane (4M, Aldrich). This mixture was allowed to stand at room temperature for 15 h, concentrated in vacuo on a rotary evaporator, and treated with an excess of 5 eq. of the theoretical amount of KOH (˜1.7 g, ˜85%, Baker) in water (20 mL) at reflux for 24 h. The mixture was cooled to room temperature and acidified with 1N HCl solution to pH 6. 3,5-Dimethylpyrazol-1-carboxamidine nitrate (1 g, Aldrich) and 0.75 g of NaHCO₃ were added and the mixture refluxed for 15 h. An additional 0.2 g carboxamidine were added, and reflux continued for 3 h, when TLC (MeOH:CHCl₃:NH₄OH (2:8:0.1) indicated complete conversion of 10-3 (lower spot) to product 10-4 (upper spot). The reaction mixture was concentrated on the rotary evaporator, and the residue slurried in a mixture of MeOH—CHCl₃—NH₄OH (3:7:0.1). Anhydrous Na₂SO₄ was added and the mixture was stirred at room temperature for 12 h, filtered and concentrated to give 3.4 g of crude guanidino acid 10-5. This material was chromatographed on silica gel (50 g), elution with MeOH—CHCl₃—NH₄OH (2:8:0.1) to give 0.65 g of 10-5, contaminated with inorganic matter (inferred from C,H,N analysis). This material was treated with concentrated HCl (0.5 mL) in absolute ethanol (10 mL) at reflux for 15 h, cooled to room temperature, concentrated, dissolved in EtOH (95:5), dried (MgSO₄), filtered and concentrated to give 0.34 g of the title compound as a hygroscopic tan powder.

NMR (400 MHz, DMSO-d₆) δ 12.78 (s, 1H), 9.15 (d, J=8 Hz, 1H), 7.93 (d, J=9 Hz, 1H), 7.84 (t, J=6 Hz, 1H), 7.37 (d, J=7 Hz, 2H), 7.32 (t, J=7 Hz, 2H), 7.25 (m, 1H), 7.1-7.6 (broad, 3H), 7.11 (s, broad 1H), 6.5 (dd, J=6 Hz, 2.6 Hz, 1H), 6.44 (d, J=2.6 Hz, 1H), 5.48 (q, A portion of an AMX, J_{AM}=4 Hz, 1H), 4.07 (t, J=5 Hz, 2H), 4.0 (m, 2H), 3.53 (m, 2H), 3.03 (q, M portion of an AMX, J_{MX}=16 Hz, 2H), 2.89(q, X portion of an AMX, J_{AX}=6 Hz, 2H), 1.08 (t, J=7 Hz, 3H); (KBr) ν (cm⁻¹) 3350, 3180, 1745, 1690; MS(+FAB) m/z 415 (M+H)⁺.

Analysis for C₂₁H₂₆N₄O₅.HCl.0.5H₂O

| | |
|---|---|
| Calculated: | C, 54.85; H, 6.14; N, 12.18. |
| Found: | C, 54.54; H, 6.04; N, 12.58. |

### EXAMPLE 280

### 3-[4-Guanidinoethoxy)-2-hydroxy-benzoylamino]-3-phenylpropanoic acid, hydrochloride (10-5)

Ester 10-4 was refluxed in 1N HCl for 15 h. The reaction was cooled and concentrated in vacuo to provide the title compound as a hygroscopic tan powder. MS (−FAB) m/z 385 (M−H)⁻; IR (KBr) ν (cm⁻¹) 3350, 3180, 1720, 1590.

Analysis for C₁₉H₂₂N₄O₅.HCl.H₂O

| | |
|---|---|
| Calculated: | C, 51.76; H, 5.72; N, 12.71 |
| Found: | C, 51.76; H, 5.74; N, 12.77. |

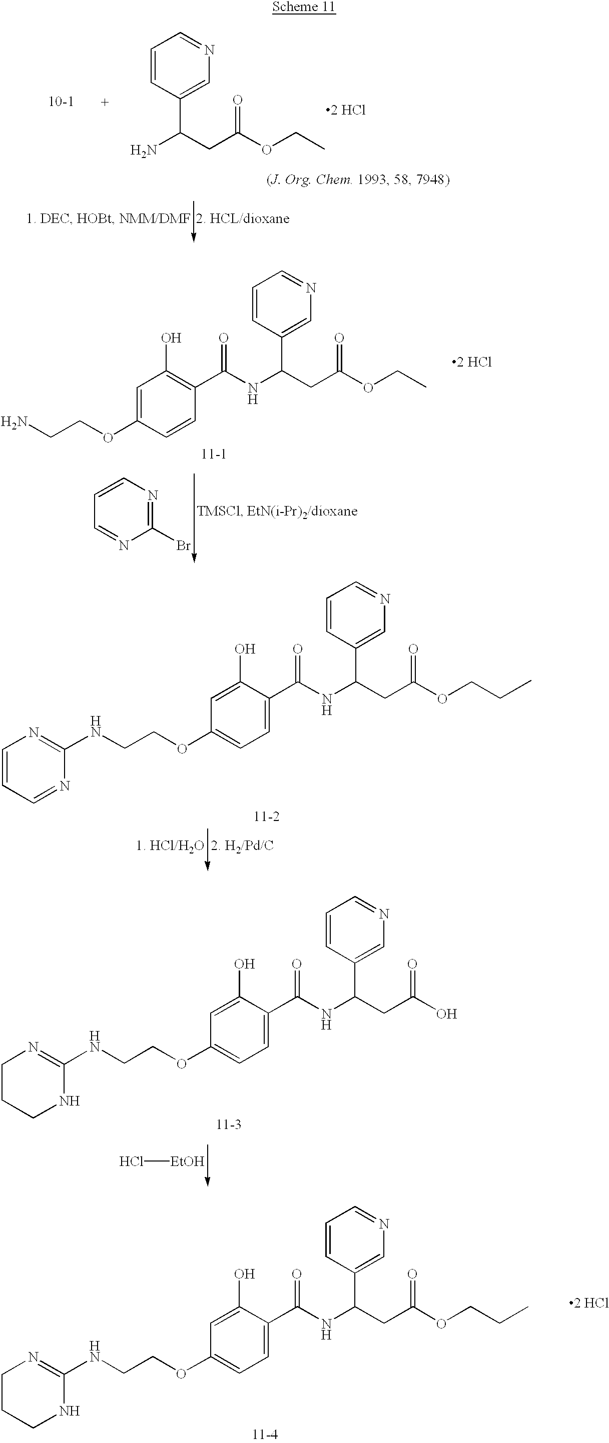

### EXAMPLE 281

### 3-[2-Hydroxy-4-[2-(pyrimidin-2ylamino)ethoxy]-benzoylamino]-3-pyridine-3-ylpropionic acid ethyl ester (11-2)

### 3-[4-(2-aminoethoxy)-2-hydroxy-benzoylamino]-3-pyridine-3-yl-propionic acid ethyl ester dihydrochloride (11-1)

Compound 10-1 (2.05 g; see Scheme 1), 3-amino-3-(pyridine-3yl, propionic acid ethyl ester dihydrocloride (1.84 g; see J. Org. Chem. 1993, 58, 7948), NMM (2.58 g), HOBt (1.16 g), and DEC (1.45 g) were combined in dichloromethane (50 mL) and stirred at room temperature for 60 h. Volatile materials were removed on the rotary evaporator, the residue partitioned between ether and H₂O, the organic phase washed with saturated aqueous brine solution, dried over MgSO₄, filtered and concentrated to give 3.52 g of crude coupled product, which was dissolved in a minimum amount of ethanol and treated with an excess of 4M HCl in anhydrous dioxane (Aldrich). After standing overnight (˜15 h), volatile materials were removed on the rotary evaporator to give 3.31 g of 11-1. NMR (400 MHz, DMSO-d₆) was consistent with the structure of 11-1; MS (+FAB) m/z 374 (M+H)⁺.

Compound 11-1 (3.31 g), 2-bromopyrimidine (1.11 g, Lancaster) and DIPEA (7.5 mL) were combined in dioxane (50 mL) at room temperature under N₂. Chlorotrimethylsilane (1.89 mL) was added and the mixture was brought to reflux. Stirring continued at this temperature for 4 days. The mixture was concentrated on the rotary evaporator, and the residue partitioned between aqueous HCl solution and chloroform. The aqueous phase was concentrated to a dark oil and the pH adjusted to 7 with aqueous ammonia. Volatile materials were removed in vacuo and the residue chromatographed on 200 g of silica gel, elution with ethyl acetate to give 1.37 g of the title compound, as an off-white powder. NMR (400 MHz, DMSO-d₆) was consistent with the structure of 11-2; (KBr) ν (cm⁻¹) 1720; MS (+FAB) m/z 452 (M+H)⁺.

Analysis for C₂₃H₂₅N₅O₅.O.5H₂O Calculated: C, 59.99; H, 5.69; N, 15.21 Found: C, 60.45; H, 5.61; N,14.79.

### EXAMPLE 282

### 3-[2-Hydroxy-4-[2-(3,4,5,6-tetrahydropyrimidine-2ylamino)-ethoxy]benzoylamino]-3-pyridin-3-ylpropionic acid (5-3)

A mixture of compound 11-2 (0.9 g) and KOH (0.34 g) were stirred in water-dioxane mixture (1:1) at room temperature. When TLC (EtOAc) showed complete absence of starting ester, solvents were removed on the rotary evaporator, 10% Pd/C (0.2 g, Aldrich) was added and the mixture suspended in acetic acid (20 mL), dioxane (10 ml), water (5 ml) and concentrated HCl (0.6 mL). The mixture was stirred at ambient temperature under H₂ atmosphere (balloon) for 2 days. Celite was added, and the mixture stirred 0.25 h, filtered through a pad of celite with the aid of dioxane-water (1:1), and concentrated. The residue was chromotographed on silica gel (25 g), elution with chloroform-methanol-ammonium hydroxide (7:3:0.1) to give the title compound 11-3 as an off-white hygroscopic powder. NMR (400 MHz, DMSO-d₆+D₂O) was consistent with the structure 11-3; IR (KBr) ν (cm⁻¹) 3400, 1650 (broad), 1580; MS (+FAB) m/z 428 (M+H)⁺.

### EXAMPLE 283

### 3-[2-Hydroxy-4-[2-(3,4,5,6-tetrahydropyrimidin -2-ylamino)ethoxy]benzoylamino]-3-pyridin-3-ylpropionic acid ethyl ester dihydrocloride (11-4)

A sample of the above zwitterion 11-3 (0.285 g) was esterified with absolute ethanol-HCl mixture at reflux. Concentration on the rotary evaporator gave the title compound 11-4. NMR (400 MHz, DMSO-d₆) δ 12.55 (s, broad, 1H), 9.5 (d, J=8 Hz, 1H), 8.94 (d, J=2 Hz, 1H), 8.73 (r, 1H), 8.48 (d, J=2 Hz, 1H), 8.73 (m, 1H), 8.48 (d, J=8 Hz, 1H), 8.1 (s, broad, 2H), 7.99 (d, J=9 Hz, 1H), 7.86 (m, 1H), 7.68 (t, J=6Hz, 1H), 6.5 (d, J=2 Hz, 1H), 6.47 (m, 1H), 5.58 (q, A portion of an AMX, J_{AM}=14 Hz, 1H), 4.08-4.0 (overlapping m, 4H), 3.5 (m, 2H), 3.25-3.19 (overlapping m, 3H), 3.09 (q, X portion of an AMX, J_{MX}=16 Hz, J_{AX}=6 Hz, 1H), 1.79 (m, 2H), 1.08(t, J=7 Hz, 3H).

Analysis for C₂₃H₂₉N₅O₅.2HCl.0.7H₂O.

| | |
|---|---|
| Calculated: | C, 51.39; H, 6.00; N, 13.03. |
| Found: | C, 51.40; H, 6.01; N, 12.56. |

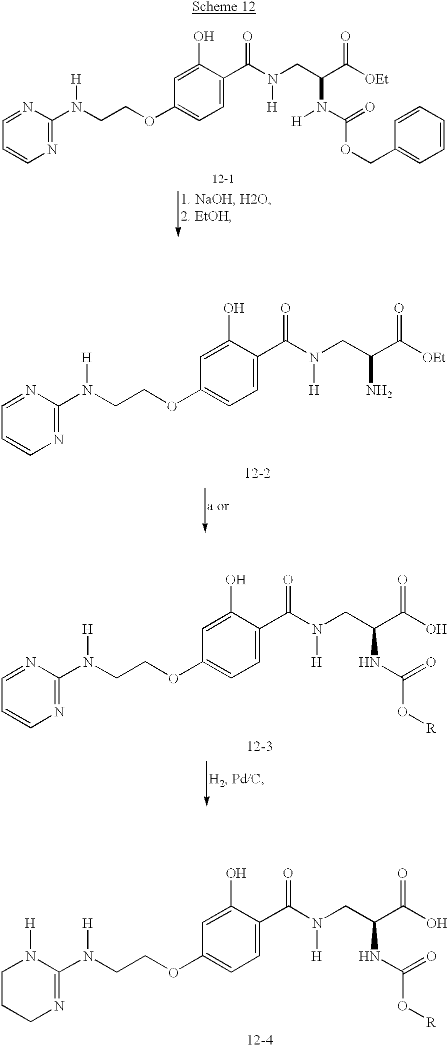

a) KOH, ₂O, then ₂O; ₁₀H₁₅(CH2ₙO(CO)O₆H₄NO₂, ₃CN, then HR-MS FAB m/z for C₂₆H₃₄N₄O₈ calcd. 531.2455 (M⁺+1), obsd. 531.2459.

### EXAMPLE 284

### 2(S)-2-tert-Butoxycarbonylamino-3-{2-hydroxy-4-[2-(1,4,5,6-tetrahydropyrimidin-2-ylamino)ethoxy]-benzoylamino}propionic acid, (12-4) acetic acid salt

Compound 12-1 (2.43 g, scheme 12; obtained as for compound 8-1, Scheme 8, by substituting 2(S)-2-benzyloxycarbonylamino-3-amino propionic acid ethyl ester (from esterification of the acid (Fluka) using EtOH-HCl) for 2(S)-2-phenylsulfonylamino-3-amino propionic acid) and NaOH (4 g) in 1,4-dioxane-water (˜1:1) were refluxed for 1.5 h. The mixture was cooled and volatile materials removed on the rotary evaporator. The residue was neutralized with aqueous 1N HCl and stirred overnight at room temperature. The precipitate was collected by vacuum filtration, re-esterified (EtOH-HCl, reflux), and chromatographed on silica gel, elution with CHCl₃,/MeOH/HOAc (90:10:1→80:20:2) to give 850 mg of 12-2, as a tan powder.

The ester 12-2 (0.5 g) was hydrolyzed with excess KOH in dioxane-H₂O at room temperature. When TLC analysis indicated an absence of starting material, an excess of di-tert-butyl dicarbonate was added and the mixture stirred at room temperature until complete by TLC. The mixture was concentrated on the rotary evaporator and the residue chromatographed on silica gel, elution with CHCl₃/MeOH/NH₄OH (90:1:1→480:20:2) to give 200 mg of 12-3. This material was dissolved in a minimum amount of acetic acid, then diluted with an ˜ volume of dioxane-H₂O (2:1). Hydrogenation (5% Pd/C (catalytic), H2, balloon, rt,) was complete within 2 days. The catalyst was filtered, and the filtrate concentrated on the rotary evaporator. The residue was stirred/concentrated sequentially with heptane and isopropanol, dissolved in CHCl₃ and treated with a mixture of activated charcoal and celite, filtered and concentrated to give the title compound 12-4 (0.18 g) as a fine buff powder. NMR (400 MHz, DMSO-d₆) δ 8.85 (broad, 1H), 8.6 (broad, 1H), 8.4 (broad, 1H), 7.68 (d, J=8.8 Hz, 1H), 6.47(overlapping peaks, 2H), 6.4 (d, J=8.8Hz, 1H) 4.02 (t, broad, 2H), 3.88 (m, 1H), 3.45 (m, overlapping, 4H), 3.23 (m, broad, 4H), 1.9 (s, 3H), 1.8 (m, broad, 2H), 1.35 (s, 9H); IR (KBr) ν (cm⁻¹) 3400 (broad), 1710, 1640; MS (ESI−) m/z 464 (M−1)+. Analysis for C₂₁H₃₁N₅O₇.HOAc.H₂O

| Calculated: | C, 50.82; H, 6.86; N, 12.88 |
|---|---|
| Found: | C, 50.96; H, 6.56; N, 12.11 |
| HPLC analysis of purity: | 96.8% |

In like manner, examples 284-315 (Table 14) were prepared, using the synthetic methods outlined above, as indicated by scheme numbers in the table. All final products were characterized as in Examples 1-283, and had spectra consistent with the assigned structures.

TABLE 14
Examples 284-315

| | |
|---|---|
| | I |
| 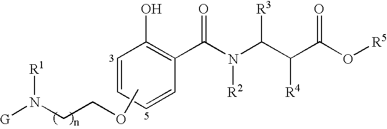 | |

| Example | | | | | | | | | Synth. | |
|---|---|---|---|---|---|---|---|---|---|---|
| No. | # | G | n | R¹ | R² | R³ | R⁴ | R⁵ | Method | Description |
| 284 | 4 | Am | 1 | H | H | Py | H | Et | 10 | brown powder |
| 285 | 4 | Am | 1 | H | H | Py | H | H | 10 | buff powder |
| 286 | 4 | Pyr | 1 | H | H | Py | H | H | 11 | yellow powder |
| 287 | 4 | Pyr | 1 | H | H | Ph | H | H | 8 | yellow powder |
| 288 | 4 | Pyr | 1 | H | H | Ph | H | Et | 8 | white wax |
| 289 | 5 | Pyr | 1 | H | H | Ph | H | H | 8 | tan powder |
| 290 | 5 | Pyr | 1 | H | H | Ph | H | Et | 8 | gold powder |
| 291 | 4 | Thp | 1 | H | H | Ph | H | H | 8 | buff powder |
| 292 | 4 | Thp | 1 | H | H | Ph | H | Et | 8 | buff powder |
| 293 | 5 | Thp | 1 | H | H | Ph | H | H | 8 | buff powder |
| 293 | 5 | Thp | 1 | H | H | Ph | H | Et | 8 | tan powder |
| 295 | 4 | Thp | 1 | H | H | H | NH₂ | H | 8 | off-white powder |
| 296 | 4 | Pyr | 1 | H | H | H | NHCbz | Me | 8 | white powder |
| 297 | 4 | Pyr | 1 | H | H | H | NHCbz | H | 8 | crystalline white powder |
| 298 | 4 | Pyr | 1 | Me | H | H | NHSO₂Ph | H | 8 | yellow powder |
| 299 | 4 | Pyr | 1 | H | H | H | NHCbz | Et | 8 | white solid mp 124-125° C. |
| 300 | 5 | Thp | 3 | H | H | Ph | H | H | 12 | buff powder |
| 301 | 5 | Pyr | 3 | H | H | Ph | H | Et | 8 | fused golden powder |
| 302 | 5 | Pyr | 3 | H | H | Ph | H | H | 8 | fine tan powder |
| 303 | 5 | Pyr | 4 | H | H | H | NHSO₂Ph | H | 8 | fine off-white powder |
| 304 | 5 | Thp | 4 | H | H | H | NHSO₂Ph | Et | 8 | fused tan solid |
| 305 | 5 | Thp | 4 | H | H | H | NHSO₂Ph | H | 8 | fine buff powder |
| 306 | 4 | Pyr | 3 | H | H | H | NHSO₂Ph | H | 8 | fine white powder |
| 307 | 4 | Thp | 3 | H | H | H | NHSO₂Ph | Et | 8 | fused tan solid |
| 308 | 4 | Thp | 3 | H | H | H | NHSO₂Ph | H | 8 | white powder |
| 309 | 5 | Thp | 3 | H | H | Ph | H | Et | 8 | off-white powder |
| 310 | 4 | Thp | 2 | H | H | H | NHSO₂Ph | i-Pr | 8^{a} | fine white powder |
| 311 | 4 | Thp | 2 | H | H | H | NHSO₂Ph | t-Bu | 8^{b} | fine off-white powder |
| 312 | 4 | Thp | 2 | H | H | H | NHSO₂Ph | (CH₂)₂O | 8^{c} | tan wax |
| | | | | | | | | (CH₂)₂NH— | | |
| | | | | | | | | BOC | | |
| 313 | 4 | Thp | 2 | H | H | H | NHCO₂— | H | 12 | fine tan powder |
| | | | | | | | CH₂CH₂C₁₀H₁₅ | | | |
| 314 | 4 | Thp | 2 | H | H | H | NHCO₂C₁₀H₁₅ | H | 12 | fin tan powder |
| 315 | 4 | Thp | 2 | H | H | H | NHSO₂Ph | H₂N—C | 8^{d} | tan powder |
| | | | | | | | | (CH₂OH)₃ | | |

Am = Amidino
Pyr = pyrimidin-2-yl
Thp = tetrahydropyrimidine
Ph = phenyl
Py = 3-pyridin-3-yl
Cbz = benzyloxycarbonyl

### Vitronectin Receptor α_{V}β₃ Binding Assay

The purpose of this assay is to measure the effect of various compounds on the α_{V}β₃—ligand interaction.

Reagents

Plasma Membrane Isolation: 15 confluent T₁₅₀ [symbol] 512P5 cells (α_{V}β₃—overexpressing cell line) are washed Dulbecco's phosphate buffered saline (D-PBS) without c [symbol] magnesium, pH 7.1. Cells are harvested with 10 mL of tryp [symbol] and collected by centrifugation. The cell pellet is washed 2X mg/mL of soybean trypsin inhibitor, and resuspended [symbol] weight/volume in homogenization buffer (25 mM Tris-HCl, [symbol] 250 mM sucrose). The cell suspension is homogenized w [symbol] seconds bursts of a Polytron homogenizer. The homog [symbol] centrifuged at 3000 g for 10 minutes at 4 C. The super [symbol] collected, measured, and made 100 mM in NaCl and 0.2 [symbol] MgSO₄. The supernatant is centrifuged at 22,000 g for 20 minu [symbol] C, the pellet is resuspended in 7 mL of membrane buffer (25 m [symbol] HCl, pH=7.4; 100 mM NaCl; 2 mM MgCl₂) by 5 strokes of a [symbol] homogenizer (tight pestle) and recentrifuged at 22,000 g for 20 [symbol] at 4 C. The pellet is resuspended in 0.5 mL/flask of membrane [symbol] (stock membranes) and frozen at −80C. Prior to use, stock mem [symbol] are Dounce homogenized and diluted 2 μL to 1000 μL in men [symbol] buffer.

Compound Dilution: The stock compounds are dissolved [symbol] appropriate vehicle (typically DMSO) and subsequently diluted in [symbol] buffer composed as follows: 25 mM Tris-HCl (pH=7.4), 100 [symbol] NaCl, 2 mM MgCl₂, 0.1% BSA.

Plate Preparation

Wells of Multiscreen-FB assay plates (Milhpore MAFB N [symbol] 50) are blocked with 150 mL of 0.1% polyethylenimine for 2 hour [symbol] 4° C. Following incubation the wells are aspirated and washed with isotonic saline solution.

Binding Assay

125 μL of assay buffer is added to each well. Next, 25 μL of labeled ligand is added to each well. 25 μL of unlabeled ligand is added to non-specific binding wells (NSB). 25 μL of assay buffer is added to all other wells. 2 μL of compound is added to appropriate sample wells, and 2 μL of DMSO is added to NSB and total binding (TB) wells. Finally, 25 μL of membrane is added to each well.

The plates are covered and incubated at 37° C. for 2 hours in a humidified incubator. Wells are aspirated on a Millipore vacuum manifold, and the wells are washed with 150 μL isotonic saline solution. Wells are again aspirated. The plates are then dried for 1 hour in an 80° C. vacuum drying oven. Plates are placed on a Millipore filter punch apparatus, and filters are placed in 12×75 mm polypropylene culture tubes. The samples are counted on a Packard gamma counter.

Example

Using ¹²⁵I-Echistatin (specific activity=2000 Ci/mmol) supplied by Amersham at a final concentration of 50 pM, the following parameters are routinely observed:

| | |
|---|---|
| Input | 80000 cpm |
| Total Counts | 8000 cpm |
| Non-specific binding | 200 cpm |

Analysis of Results

The individual well activity is expressed as a percentage of the specific binding; % Max, and reported as the mean±standard deviation. Dose-inhibition relationships are generated for dose (X-axis) vs. % Max (Y-axis) for active compounds using a non-linear regression computer program (PS-NONLIN), and IC₅₀ values with corresponding 95% confidence intervals are estimated from 50% of maximal attachment. Results are shown in Table 15 (VnR).

Reference Compounds

Various Arginine-Glycine-Aspartic Acid (RGD)-containing peptides were assessed for the ability to inhibit a_{V}b₃ binding and the corresponding IC₅₀ values with 95% confidence intervals were generated; peptide structures are given by the standard single letter designation for amino acids. Values obtained compared favorably with adhesion assay results.

| Peptid | IC₅₀ (μM) | 95% Confidence Interval |
|---|---|---|
| GPenGRGDSPCA | 0.064 | 0.038 to 0.102 |
| GRGDSP | 1.493 | 1.058 to 2.025 |
| GRGDTP | 0.490 | 0.432 to 0.556 |
| GRGDS | 0.751 | 0.690 to 0.817 |
| RGDS | 1.840 | 1.465 to 2.262 |
| GRGDNP | 0.237 | 0.144 to 0.353 |
| GdRGDSP | 0.692 | 0.507 to 0.942 |
| GRGESP | inactive at 100 μM | |

References

1. Nesbitt, S. A. And M. A. Horton, (1992), A nonradioactive biochemical characterization of membrane proteins using enhanced chemiluminescence, Anal. Biochem., 206 (2), 267-72.

### Osteopontin-α_{V}β₃ Cell Attachment Assay

The purpose of this assay is to measure the effect of various compounds on the RGD-dependent attachment of cells to osteopontin mediated by the α_{V}β₃ integrin.

Reagents

Cell Suspension Media: The cells are suspended for assay in the tissue culture media used for normal culture maintenance buffered with 25 mM HEPES (pH 7.4) without serum supplementation.

Compound Dilution Media: The stock compounds are dissolved in an appropriate vehicle (typically DMSO) and subsequently diluted in the tissue culture media used for normal culture maintenance buffered with 25 mM HEPES (pH 7.4) supplemented with 0.2% BSA (no serum); final vehicle concentration is ≦0.5%.

Plate Preparation

Human recombinant osteopontin (prepared such as described in Stubbs, J. III, Connective Tissue Research, 1996, 35 (1-4), 393-399 is diluted to an appropriate concentration in Dulbecco's phosphate buffered saline (D-PBS) without calcium or magnesium, pH 7.1. 100 mL of this solution is incubated in the wells of PRO-BIND assay plates (Falcon 3915) for 2 hours at 37° C. Following incubation the wells are aspirated and washed once with D-PBS; plates can either be used immediately or stored for up to 1 week at 4° C. Prior to assay, the wells are blocked with 1% bovine serum albumin (BSA) in cell suspension media for 1 hour at 37° C. Following the blocking period, wells are aspirated and washed once with D-PBS.

Cell Suspension

α_{V}β₃-expressing cell lines are maintained by standard tissue culture techniques. For assay, the cell monolayer is washed three times with D-PBS, and the cells are harvested with 0.05% trypsin0.53 mM EDTA (GIBCO). The cells are pelleted by low-speed centrifugation and washed three times with 0.5 mg/mL trypsin inhibitor in D-PBS (Sigma). The final cell pellet is resuspended in cell suspension media at a concentration of 10⁶ cells/mL.

Attachment Assay

Incubation: 100 mL of diluted test compound is added to osteopontin-coated wells (in triplicate) followed by 100 mL of cell suspension; background cell attachment is determined in uncoated wells. The plate is incubated at 25° C. in a humidified air atmosphere for 1.5 hours. Following the incubation period, the wells are gently aspirated and washed once with D-PBS.

Cell Number Detection: The number of cells attached is determined by an MTT dye conversion assay (Promega) according to the manufacturer's instructions. Briefly, MTT dye is diluted in cell suspension media (15:85) and 100 mL is added to each well. The assay plates are incubated for 4 hours at 37° C. in a humidified 5% CO₂/95% air atmosphere, followed by the addition of 100 mL stopping/solubilization solution. The assay plates are covered and incubated at 37° C. in a humidified air atmosphere overnight. After the solubilization period, the optical density of the wells is measured at a test wavelength of 570 nM with a reference measurement taken simultaneously at 630 nM.

Analysis of Results

The individual well optical density is expressed as a percentage of the maximal attachment (% Max) wells minus background attachment, and reported as the mean±standard deviation. Dose-inhibition relationships are generated for dose (X-axis) vs. % Max (Y-axis) for active compounds using a non-linear regression computer program (PS-NONLIN), and IC₅₀ values with corresponding 95% confidence intervals are estimated from 50% of maximal attachment. Results are shown in Table 16 (“cell”).

Reference Compounds

Various Arginine-Glycine-Aspartic Acid (RGD)-containing peptides, and monoclonal antibodies (Chemicon, Temecula, Calif.) were assessed for the ability to inhibit osteopontin-a_{V}b₃ attachment and the corresponding IC₅₀ values with 95% confidence intervals were generated in the SK-MEL-24 human malignant melanoma cell line; peptide structures are given by the standard single letter designation for amino acids:

| Peptide | IC₅₀ (95% Confidence Interval) |
|---|---|
| GPenGRGDSPCA | 0.58 mM (0.51 TO 0.67) |
| n-Me-GRGDSP | 4.0 mM (3.4 TO 4.7) |
| GRGDSP | 4.1 mM (3.4 TO 4.9) |
| GRGDTP | 5.2 mM (3.4 TO 4.9) |

| | | % Maximal Attachment |
|---|---|---|
| Antibody | Dilution | (mean ± SD) |
| αᵥβ₅ (P1F6) | 1:1000 | 111 ± 3.3 |
| | 1:100 | 112 ± 2.6 |
| | 1:10 | 111 ± 3.3 |
| αᵥβ₃ (LM609) | 1:1000 | 0 |
| | 1:100 | 5.1 ± 1.7 |

Literature References

Ruoslahti, R. Fibronectin and its receptors. Ann. Rev. Biochem. 57:375-413, 1988.

Hynes, R. O. Integrins: Versatility, modulation, and signaling in cell adhesion. Cell. 69: 11-25, 1992.

### Osteoclast Pitting Assay

The assay is conducted as described in Murrills and Dempster (1990). Briefly, 4×4×0.2 mm slices of devitalized bovine cortical bone are numbered, placed in the wells of 96-well culture plates and wetted with 100 ul of Medium 199 containing Hanks salts, 10 mM HEPES, pH 7.0 (Medium 199/Hanks). Bone cell suspensions containing osteoclasts are prepared by mincing the long bones of neonatal rats (Sprague-Dawley, 4-6 days old) in Medium 199/Hanks. 100 uL of the suspension is then plated onto each slice and incubated 30 minutes to allow osteoclasts to adhere. The slices are rinsed to remove non-adherent cells and incubated 24 h in Medium 199 containing Earle's salts, 10 mM HEPES and 0.7 g/L NaHCO₃, which equilibrates at pH 6.9 in a 5% CO₂ atmosphere. At this pH the adherent osteoclasts excavate an adequate number of resorption pits for assay purposes. Slices are fixed in 2.5% glutaraldehyde and osteoclasts counted following tartrate-resistant acid phosphatase staining. In experiments in which osteoclast numbers are significantly reduced in a particular treatment, a check is made for non-specific cytotoxicity by counting the number of contaminant fibroblast-like cells following toluidine staining. All cells are stripped from the slice by sonication on 0.25M NH₄OH and the resorption pits formed by the osteoclasts during the experiment stained with toluidine blue. Resorption pits are quantified by manually counting.

Statistics

The experiments are conducted according to a block design with osteoclasts from each animal exposed to each treatment. Three replicate slices are used per treatment per animal, such that a total of 96 slices are examined for an experiment involving four animals and eight treatments (including control). Several parameters are recorded on a “per slice” basis: number of pits, number of osteoclasts, number of pits per osteoclast, number of fibroblast-like bone cells. SAS or JMP statistical software is used for statistical analysis. If analysis of variance reveals significant effects in the experiment, those treatments differing significantly from control are identified using Dunnett's test. IC₅₀s are calculated for active compounds using dose-response curves. Results are shown in Table 16 (“Bone Pitting”).

Reference Compound: Rat calcitonin.

Clinical Relevance:

Osteoclasts are responsible for the bone loss that occurs in the onset of osteoporosis and anti-resorptive drugs directed against the osteoclast are a requirement for patients losing bone. Calcitonin and bisphosphonates, both used as anti-resorptives in the clinic, show significant osteoclast inhibitory activity in this assay. Hence it is a reasonable assay in which to identify novel anti-resorptives.

Reference: Murrills and Dempster (1990) Bone 11:333-344.

### Effects of test compounds on PTH-induced hypercalcemia of thyroparathyroidectomized male rats

Male thyro-parathyroidectomized (TPTX) rats (Charles River) were randomly assigned to groups of 7 rats/group. Following a baseline serum calcium determination an Alzet 1003D minipump (Alza Corporation, Palo Alto, Calif.) loaded with 0.3 mg/ml PTH (Bachem, Philadelphia, Pa.) was implanted subcutaneously in each rat. For evaluation of prophylactic effects of a test drug, another minipump with appropriate concentration of the test drug solution was implanted subcutaneously at a site away from PTH minipump. Alternatively, test drugs were administered by oral gavage as a solution or uniform suspension in an appropriate medium depending on the physical properties of the test compound. A group of 7 unimplanted TPTX rats was set aside as a normal control group. Twenty hours after minipump implantation blood was collected from each rat to confirm the presence of hypercalcemia (judged by elevation of serum calcium levels, 2 SD>normal non-implanted level). At various intervals between 0.5 and 24 hours after dosing (usually one to three time points), blood was collected from each rat and the serum evaluated for total calcium. Serum calcium levels were measured using the Nova 7+7 calcium auto analyzer spectrophotometrically using the Sigma test kit (#587A). Test results were determined by the difference in serum calcium between vehicle and treatment group following PTH administration, using a one-way analysis of variance with Dunnett's test or other multiple comparison methods. Results are shown in Table 17.

References:

1. Takeuchi M, Sakamoto S, Kawamuki K, Kudo M, Abe T, Fujita S, Murase K, and Isomura Y, (1990). Synthesis and structure activity relationship of new bisphosphonate derivative. Abstract #53, 199^{th} American Chemical Society Meeting Boston, Mass.

2. Fisher J. Caulfield M, Sato M, Quartuccio H, Gould R, Garsky V, Rodan G, Rosenblatt M, (1993). Inhibition of osteoclastic bone resorption in vivo by echistatin, an “arginyl-glycyl-aspartyl” (RGD)-containing protein. Endocrinology, Vol. 132 (3) 1411-1413.

TABLE 15
Representative Biological Data

| Example | VnR (IC₅₀μM) |
|---|---|
| 1 | 0.0241 |
| 2 | 0.187 |
| 3 | 0.123 |
| 4 | 0.095 |
| 5 | 0.061 |
| 6 | 0.108 |
| 7 | 0.092 |
| 8 | >1 uM |
| 9 | 0.11 |
| 10 | 0.061 |
| 11 | 0.0696 |
| 12 | 0.0661 |
| 13 | 0.1828 |
| 14 | 0.0445 |
| 15 | |
| 16 | |
| 17 | |
| 18 | |
| 19 | |
| 20 | 1.437 |
| 21 | 1.516 |
| 22 | |
| 23 | 1.0216 |
| 24 | 1.48 |
| 25 | 0.6743 |
| 26 | |
| 27 | 0.3308 |
| 28 | 0.159 |
| 29 | 0.405 |
| 30 | 1.27 |
| 31 | 0.261 |
| 32 | |
| 33 | |
| 34 | |
| 35 | |
| 36 | |
| 37 | |
| 38 | |
| 39 | |
| 40 | |
| 41 | |
| 42 | |
| 43 | |
| 44 | |
| 45 | |
| 46 | |
| 47 | |
| 48 | |
| 49 | |
| 50 | |
| 51 | |
| 52 | |
| 53 | 34 |
| 54 | 34 |
| 55 | 100.6 |
| 56 | 85.8 |
| 57 | |
| 58 | 100 |
| 59 | 100 |
| 60 | |
| 61 | |
| 62 | 100 |
| 63 | |
| 64 | |
| 65 | 100 |
| 66 | 100 |
| 67 | 100 |
| 68 | 100 |
| 69 | 100 |
| 70 | 100 |
| 71 | >1 uM |
| 72 | >1 uM |
| 73 | >1 uM |
| 74 | >1 uM |
| 75 | |
| 76 | |
| 77 | |
| 78 | >1 uM |
| 79 | >1 uM |
| 80 | >1 uM |
| 81 | >1 uM |
| 82 | >1 uM |
| 83 | >1 uM |
| 84 | |
| 85 | >1 uM |
| 86 | >1 uM |
| 87 | >1 uM |
| 88 | >1 uM |
| 89 | |
| 90 | >1 uM |
| 91 | >1 uM |
| 92 | >1 uM |
| 93 | >1 uM |
| 94 | >1 uM |
| 95 | 0.105 |
| 96 | 0.119 |
| 97 | 0.77 |
| 98 | 0.15 |
| 99 | 0.088 |
| 100 | 0.079 |
| 101 | 0.094 |
| 102 | 0.069 |
| 103 | 0.21 |
| 104 | 0.086 |
| 105 | 0.135 |
| 106 | 0.114 |
| 107 | 0.13 |
| 108 | 1.105 |
| 109 | 0.251 |
| 110 | 0.544 |
| 111 | 0.856 |
| 112 | 1.092 |
| 113 | 3.026 |
| 114 | 3.139 |
| 115 | 0.258 |
| 116 | 2.761 |
| 117 | 1.518 |
| 118 | >1 uM |
| 119 | >1 uM |
| 120 | >1 uM |
| 121 | >1 uM |
| 122 | >1 uM |
| 123 | >1 uM |
| 124 | 0.734 |
| 125 | >1 uM |
| 126 | >1 uM |
| 127 | 0.9546 |
| 128 | >1 uM |
| 129 | 0.6349 |
| 130 | >1 uM |
| 131 | 0.4055 |
| 132 | 0.9625 |
| 133 | >1 uM |
| 134 | >1 uM |
| 135 | >1 uM |
| 136 | >1 uM |
| 137 | >1 uM |
| 138 | 0.361 |
| 139 | >1 uM |
| 140 | 0.0978 |
| 141 | >1 uM |
| 142 | 1.6 |
| 143 | 4.79 |
| 144 | |
| 145 | |
| 149 | |
| 150 | |
| 151 | |
| 152 | |
| 153 | 2.4 |
| 154 | |
| 155 | |
| 156 | 0.25 |
| 157 | |
| 158 | 4.6 |
| 159 | 2 |
| 160 | 0.97 |
| 161 | 0.9 |
| 162 | 1.1 |
| 163 | 1.1 |
| 164 | 0.61 |
| 165 | 0.39 |
| 166 | 0.8 |
| 167 | 2.6 |
| 168 | |
| 169 | |
| 170 | |
| 171 | |
| 172 | |
| 173 | 4.28 |
| 174 | 3.89 |
| 175 | 3.8 |
| 176 | 2.14 |
| 177 | 4.87 |
| 178 | 3.13 |
| 179 | >1 uM |
| 180 | 19.46 |
| 181 | 19.72 |
| 182 | 40.88 |
| 183 | 4.98 |
| 184 | 17.88 |
| 185 | 4.57 |
| 186 | 6.99 |
| 187 | 19.46 |
| 188 | 12.14 |
| 189 | 6.87 |
| 190 | >1 uM |
| 191 | >1 uM |
| 192 | >1 uM |
| 193 | >1 uM |
| 194 | >1 uM |
| 195 | >1 uM |
| 196 | 5.7127 |
| 197 | >1 uM |
| 198 | >1 uM |
| 199 | 14.694 |
| 200 | >1 uM |
| 201 | 13.215 |
| 202 | >1 uM |
| 203 | 14.136 |
| 204 | 7.4788 |
| 205 | >1 uM |
| 206 | >1 uM |
| 207 | >1 uM |
| 208 | >1 uM |
| 209 | >1 uM |
| 210 | >1 uM |
| 211 | >1 uM |
| 212 | 13.066 |
| 213 | >1 uM |
| 214 | 2.3125 |
| 215 | >1 uM |
| 216 | |
| 217 | |
| 218 | |
| 219 | 1.8 |
| 220 | 8.8 |
| 221 | 22.8 |
| 222 | 20.8 |
| 223 | 5.5 |
| 224 | 4.1 |
| 225 | 5 |
| 226 | 5.2 |
| 227 | 5.1 |
| 228 | 10.2 |
| 229 | 17.1 |
| 230 | 4 |
| 231 | 6.7 |
| 232 | 3.7 |
| 233 | 3.3 |
| 234 | 8.7 |
| 235 | 3 |
| 236 | 1.9 |
| 237 | 2.7 |
| 238 | |
| 239 | 2.1 |
| 240 | |
| 241 | 4.1 |
| 242 | 7.9 |
| 243 | 0.69 |
| 244 | 1.6 |
| 245 | |
| 246 | 1.6 |
| 247 | 5.5 |
| 248 | |
| 249 | |
| 250 | |
| 251 | |
| 252 | |
| 253 | |
| 254 | |
| 255 | 5.31 |
| 256 | 9.08 |
| 257 | 1.26 |
| 258 | 4.31 |
| 259 | |
| 260 | |
| 261 | 20.18 |
| 262 | 12.64 |
| 263 | 29.03 |
| 264 | 59.27 |
| 265 | 12.88 |
| 266 | 29.57 |
| 267 | 10.16 |
| 268 | 33.44 |
| 269 | 21.23 |
| 270 | 21.66 |
| 271 | 13.7 |
| 272 | 10.63 |
| 273 | |
| 274 | |
| 275 | |
| 276 | |
| 277 | |
| 278 | |
| 279 | 0.013 |
| 280 | 12.3 |
| 281 | inactive |
| 282 | −42% @ 100 |
| 283 | 123 |
| 284 | inactive |
| 285 | 5 |
| 286 | 2.8 |
| 287 | 0.26 |
| 288 | 0.003 |
| | bone pitting IC50 = 0.44 μM |
| 289 | inactive |
| 290 | 0.334 |
| 291 | 0.44 |
| 292 | 0.115 |
| 293 | 0.006 |
| 294 | 0.0035 |
| 295 | 0.0018 |

TABLE 16
In Vitro Biological Data

| Example | IC₅₀ (μM) | |
|---|---|---|
| No. | Cell^{A} | Bone Pitting^{B} |
| 280 | 78 | inactive @ 200 |
| 297 | 50 | 25 |
| 277 | 0.05 | 0.4 |
| 278 | 0.02 | 0.5 |
| 274 | 18 | 0.9 |
| 293 | 48 | |
| 276 | 0.12 | 0.15 |
| 291 | 28 | |
| 275 | 0.002 | 0.43 |
| 299 | inactive @ 100 | 1.9 |
| 298 | | |
| 285 | 56 | |
| 286 | 86 | |
| 282 | 33 | |
| 289 | 34 | |

^{A}Osteopontin - αᵥβ₃ Cell Attachment Assay
^{B}Osteoclast Pitting Assay

TABLE 17
In Vivo Biological Data

| Example No. | TPTX (% inhibition) | dose (mg/kg, route) |
|---|---|---|
| 292 | 111* | 100, s.c. |
| 279 | 59 | 100, s.c.. |
| 273 | 57 | 100, s.c. |
| 277 | 86* | 100, s.c. |
| | 79* | 100, p.o. |
| 276 | 170* | 100, s.c. |
| 274 | 54* | 100, s.c. |
| 275 | 112* | 100, s.c. |
| | (64) | 30, s.c. |
| | 105* | 75, s.c. |
| | 39 | 100, p.o. |
| 291 | 41 | 100, s.c. |
| 299 | 102* | 100, p.o. |

*p < 0.05 when compared to vehicle control

The compounds of the present invention can be used in the form of salts derived from pharmaceutically or physiologically acceptable acids or bases. These salts include, but are not limited to, salts with inorganic acids such as hydrochloric acid, sulfuric acid, nitric acid, phosphoric acid and salts with organic acids such as acetic acid, oxalic acid, succinic acid, and maleic acid. Other salts include salts with alkali metals or alkaline earth metals, such as sodium, potassium, calcium or magnesium. The compounds of the present invention can also be used in the form of esters at the C-terminus; carbamates, amides and the like at the N-terminus or other conventional “pro-drug” forms which, when administered, convert to the active moiety in vivo.

Compounds of the present invention may be administered in combination with one or more pharmaceutically acceptable carriers, for example, solvents, diluents and the like. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils. Adjuvents customarily employed in the preparation of pharmaceutical compositions may be advantageously included, such as flavoring agents, coloring agents, preserving agents, and antioxidants, for example, vitamin E, ascorbic acid, BHT and BHA. These compounds may be administered orally as well as by intravenous, intramuscular, or subcutaneous routes. When administered orally in such forms as tablets, capsules, dispersible powders, granules, or suspensions, formulations may contain, for example, from about 0.05 to 5% of suspending agent, syrups containing, for example, from about 10 to 50% of sugar, or elixirs containing, for example, from about 20 to 50% ethanol, and the like. When administration is parenterally, formulation may be, for example, sterile injectable solutions or suspensions containing from about 0.05 to 5% suspending agent in an isotonic medium. Such pharmaceutical preparations may contain, for example, from about 25 to about 90% by weight of active ingredient in combination with a carrier, and more preferably between about 5% and 60% by weight of active ingredient.

The preferred pharmaceutical compositions from the standpoint of ease of preparation and administration are solid compositions, particularly tablets and hard-filled or liquid-filled capsules. Oral administration of the compounds is preferred.

The effective dosage of active ingredient employed may vary depending on the particular compound employed, the mode of administration and the severity of the condition being treated. However, in general, satisfactory results are obtained when compounds of the invention are administered at a daily dosage of from about 0.5 to about 500 mg/kg of animal body weight, preferably given in divided doses two to four times a day, or in a sustained release release form. Preferably, the total daily dosage is from about 1 to 100 mg, preferably from about 2 to 80 mg. Dosage forms suitable for internal use comprise from about 0.5 to 500 mg of active compound in intimate admixture with a solid or liquid pharmaceutically acceptable carrier. This dosage regimen may be adjusted to provide the optimal therapeutic response as would be appreciated by one skilled in the art. For example, several divided doses may be administered daily or the dose may be proportionally reduced as indicated by the exigencies of the therapeutic situation.

What is claimed is:

1. A compound of the formula 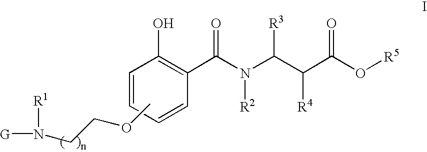
wherein:
G is 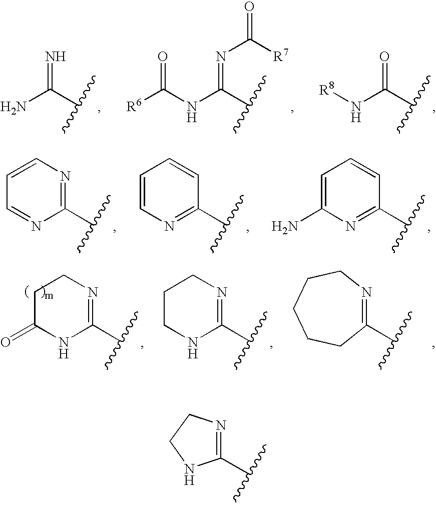
R¹ and R² are independently, hydrogen, alkyl of 1 to 6 carbon atoms, mono or bicyclic aralkyl of 6 to 10 carbon atoms, or heterocycloalkyl-alkyl comprised of a 5 to 10 membered mono or bicyclic heterocycloalkyl having 1 to 3 heteroatoms selected from S, N and O and an alkyl of 1 to 6 carbon atoms;
R³ is hydrogen, mono or bicyclic aryl of 6 to 10 carbon atoms, 5 to 10 membered mono or bicyclic heterocycloalkyl having 1 to 3 heteroatoms selected from S, N and O;
R⁴ is hydrogen, NHR⁹, OR⁹, NHCO₂R⁹, NHCONHR⁹, NHCOR⁹ or NHSO₂R⁹; provided that R³ and R⁴ are not both hydrogen;
R⁵ is hydrogen or alkyl of 1 to 6 carbon atoms;
R⁶ and R⁷ are independently hydrogen, alkyl of 1 to 6 carbon atoms, alkoxy of 1 to 6 carbon atoms, or aralkoxy of 6 to 10 carbon atoms;
R⁸ and R⁹ are independently hydrogen, trichoroalkylalkoxy, trifluoromethoxyphenyl, arakenyl of 7 to 10 carbon atoms, alkyl of 1 to 10 carbon atoms, alkenyl of 2 to 10 carbons, alkynyl of 2 to 10 carbons, mono or polycycloalkyl of 3-12 carbon atoms, mono or polycycloalkyl-alkyl of 4-12 carbon atoms, mono or bicyclic aryl of 6 to 10 carbon atoms, 6 to 10 membered mono or bicyclic heterocycloalkyl having 1 to 3 heteroatoms selected from S, N and O, mono or bicyclic aralkyl of 7 to 10 carbon atoms, or heterocycloalkyl-alkyl comprised of a 5 to 10 membered mono or bicyclic heterocycloalkyl having 1 to 3 heteroatoms selected from S, N and O and an alkyl of 1 to 6 carbon atoms;
n is an integer from 1 to 4; and m is 0 or 1; or a pharmaceutically acceptable salt thereof.

2. A compound of claim 1 wherein G is 6-aminopyridin-2yl, pyridin-2yl, pyrimidin-2yl, tetrahydropyrimidin-2yl, tetrahydropyrimid-4-one-2yl, imidazol-2yl, 5-amino 1,2,4-triazol-3-yl, dihydroimidazol-2yl, amino(imino)methyl, pyridyl-NHC(═O)—, or benzyl-NHC(═O)—.

3. A compound of claim 2 wherein G is pyridin-2yl, imidazolyl-2yl, 5-amino 1,2,4-triazol-3-yl, or tetrahydropyrimidin-2yl.

4. A compound of claim 1 wherein R¹ and R² are hydrogen.

5. A compound of claim 1 wherein R³ is hydrogen, phenyl, or pyridyl.

6. Compound of claim 1 wherein R⁴ is hydrogen, HNSO₂phenyl, HNSO₂alkyl, NHCO₂benzyl, NHCO₂neopentyl, NHCO₂adamantyl, NHCO₂CH₂adamantyl, NHCONHphenyl, NHCONHbenzyl, NHCONHalkyl, NHCOphenyl, NHCObenzyl, NHCOalkyl or NHCOadamantyl.

7. A compound of claim 1 wherein G is pyridin-2yl, 6-aminopyridin-2yl, 5-amino 1,2,4-triazol-3-yl, imidazol-2yl, dihydroimidazol-2yl or tetrahydropyrimidin-2yl, R¹, R² and R³ are hydrogen, R⁴is NHSO₂phenyl, NHCO₂benzyl or NHCO₂neopentyl and R⁵ is hydrogen or alkyl of 1 to 3 carbon atoms.

8. A compound of claim 1 wherein G is pyridin-2yl, 6-aminopyridin-2yl, 5-amino 1,2,4-triazol-3-yl, imidazol-2yl, dihydroimidazol-2yl, or tetrahydropyrimidin-2yl, R¹, R² and R⁴ are hydrogen, R³ is phenyl or pyridyl and R⁵ is hydrogen or alkyl of 1 to 4 carbon atoms.

9. A compound of claim 1 wherein n is 2 or 3.

10. A compound of claim 1 where G is 1,4,5,6-tetrahydropyrimidyl and R⁴ is NHCO₂R⁹.

11. A compound of claim 1, which is:
(2S)-3-({2-hydroxy-4-[2-(1,4,5,6-tetrahydropyrimidin-2-ylamino)ethoxy]benzoyl}-amino)-2-[(methoxycarbonyl)amino]propanoic acid;
(2S)-2-[(ethoxycarbonyl)amino]-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]benzoyl}-amino)2-[(propxycarbonyl)amino]propanoic acid;
(2S)-3-({2-hydroxy-4-[2-(1,4,5,6-tetrahydropyrimidin-2-ylamino)ethoxy]benzoyl}-amino)2-[(isopropxycarbonyl)amino]propanoic acid;
(2S)-2-{[(allyloxy)carbonyl]amino}-3-({2-hydroxy-4-[2-(1,4,5,6-tetrahyrdropyrimidin-2-ylamino)ethoxy]benzoyl}amino)propanoic acid;
(2S)-2-{[(but-3-enyloxy)carbonyl]amino}-3-({2-hydroxy-4-[2-(1,4,5,6-tetrahydropyrimidin-2-ylamino)ethoxy]benzoyl}amino)propanoic acid;
(2S)-2-{[(hexyloxy)carbonyl]amino}-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]benzoyl}-amino)-2-{[(octyloxy)carbonyl]amino}propanoic acid;
(2S)-3-({2-hydroxy-4-[2-(1,4,5,6-tetrahyrdropyrimidin-2-ylamino)ethoxy]benzoyl}-amino)-2-{[(neopentyloxy)carbonyl]amino}propanoic acid;
(2S)-3-({2-hydroxy-4-[2-(1,4,5,6-tetrahydropyrimidin-2-ylamino)ethoxy]benzoyl}-amino)-2-{[(2,2,2-trichloroethoxy)carbonyl]amino}propanoic acid;
(2S)-3-({2-hydroxy-4-[2-(1,4,5,6-tetrahydropyrimdin-2-ylamino)ethoxy]benzoyl}-amino)-2-[(butoxycarbonyl)amino]propanoic acid;
(2S)-3-({2-hydroxy-4-[2-(1,4,5,6-tetrahydropyrimidin-2-ylamino)ethoxy]benzoyl}-amino)-2-[(isobutoxycarbonyl)amino]propanoic acid;
(2S)-3-({2-hydroxy-4-[2-(1,4,5,6-tetrahyrdropyrimidin-2-ylamino)ethoxy]benzoyl}-amino)-2-{[(prop-2-ynyloxy)carbonyl]amino}propanoic acid; or
(2S)-2-{[(benzyloxy)carbonyl]amino}-3-({2-hydroxy-4-[2-(1,4,5,6-tetrahyrdropyrimidin-2-ylamino)ethoxy]benzoyl}amino)propanoic acid, or a pharmaceutically acceptable salt form thereof.

12. A compound of claim 1 wherein G is 1,4,5,6-tetrahydropyrimidyl and R⁴ is NHCONHR⁹.

13. A compound of claim 1 which is:
(2S)-2-{[(butylamino)carbonyl)amino}-3-(2{2-hydroxy-4-[2-(1,4,5,6-tetrahydropyrimidin-2-ylamino)ethoxy]benzoyl}amino)propanoic acid;
(2S)-2-{[(hexylamino)carbonyl]amino}-3-({2-hydroxy-4-[2-(1,4,5,6-tetrahydropyrimidin-2-ylamino)ethoxy]benzoyl}amino)propanoic acid;
(2S)-3-({2-hyrdoxy-4-[2-(1,4,5,6-tetrahydropyrimidin-2-ylamino)ethoxy]benzoyl}-amino)-2-{[(octylamino)carbonyl]amino}propanoic acid;
(2S)-2-{[(allylamino)carbonyl]amino}-3-({2-hydroxy-4-[2-(1,4,5,6-tetrahydropyrimidin-2-ylamino)ethoxy]benzoyl}amino)propanoic acid;
(2S)-2-{[(1-adamantylamino)carbonyl]amino}-3-({2-hydroxy-4-[2-(1,4,5,6-tetrahydropyrimidin-2-ylamino)ethoxy]benzoyl}amino)propanoic acid;
(2S)-2-[(anilinocarbonyl)amino]-3-({2-hydroxy-4-[2-(1,4,5,6-tetrahydropyrimidin-2-ylamino)ethoxy]benzoyl}amino)propanoic acid;
(2S)-2-{[(cyclohexylamino)carbonyl]amino}-3-({2-hydroxy-4-[2-(1,4,5,6-tetrahydropyrimidin-2-ylamino)ethoxy]benzoyl}amino)propanoic acid;
(2S)-2-{[(benzylamino)carbonyl]amino}-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]-benzoyl}amino-2-[(4-toluidinocarbonyl)amino]propanoic acid;
(2S)-3-({2-hydroxy-4-[2-(1,4,5,6-tetrahydropyrimidin-2-ylamino)ethoxy]-benzoyl}amino)-2-[(2-toluidinocarbonyl)amino]propanoic acid;
(2S)-3-({2-hydroxy-4-[2-(1,4,5,6-tetrahydropyrimidin-2-ylamino)ethoxy]-benzoyl}amino)-2-{[(2-methoxyanilino)carbonyl]amino}propanoic acid;
(2S)-3-({2-hydroxy-4-[2-(1,4,5,6-tetrahydropyrimidin-2-ylamino)ethoxy]benzoyl}-amino)-2-{[(4-methoxyanilino)carbonyl]amino}propanoic acid;
(2S)-2-{[(2-chloroanilino)carbonyl]amino}-3-({2-hydroxy-4-[2-(1,4,5,6-tetrahydropyrimidin-2-ylamino)ethoxy]benzoyl}amino)propanoic acid;
(2S)-2-{[(2-bromoanilino)carbonyl]amino}-3-({2-hydroxy-4-[2-(1,4,5,6-tetrahydropyrimidin-2-ylamino)ethoxy]benzoyl}amino)propanoic acid;
(2S)-2-{[([1,1′-biphenyl]-2-ylamino)carbonyl]amino}-3-({2-hydroxy-4-[2-(1,4,5,6-tetrahydropyrimidin-2-ylamino)ethoxy]benzoyl}amino)propanoic acid;
(2S)-2-{[(4-chloroanilino)carbonyl]amino}-3-({2-hydroxy-4-[2-(1,4,5,6-tetrahydropyrimidin-2-ylamino)ethoxy]benzoyl}amino)propanoic acid; or
(2S)-3-({2-hydroxy-4-[2-(1,4,5,6-tetrahydropyrimidin-2-ylamino)ethoxy]benzoyl}-amino)-2-{[(1-naphthylamino)carbonyl]amino}propanoic acid;
or a pharmaceutically acceptable salt form thereof.

14. A compound of claim 1 wherein G is 1,4,5,6-tetrahydropyrimidyl and R⁴ is NHCOR⁹.

15. A compound of claim 1 which is:
(2S)-3-({2-hydroxy-4-[2-(1,4,5,6-tetrahydropyrimidin-2-ylamino)ethoxy]benzoyl}-amino)-2-({[(2-phenylethyl)amino]carbonyl}amino)propanoic acid;
(2S)-3-({2-hydroxy-4-[2-(1,4,5,6-tetrahydropyrimidin-2-ylamino)ethoxy]benzoyl}-amino)-2-(isobutyrylamino)propanoic acid;
(2S)-2-(hexanoylamino)-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]benzoyl}-amino)-2-(pentanoylamino)propanoic acid;
(2S)-2-[(3,3-dimethylbutanoyl)amino]-3-({2-hydroxy-4-[2-(1,4,5,6-tetrahydropyrimidin-2-ylamino)ethoxy]benzoyl}amino)propanoic acid;
(2S)-2-[(cyclohexylcarbonyl)amino]-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]benzoyl}-amino)-2-[(2-phenylacetyl)amino]propanoic acid;
(2S)-3-({2-hydroxy-4-[2-(1,4,5,6-tetrahydropyrimidin-2-ylamino)ethoxy]benzoyl}-amino)2-[(3-phenylpropanoyl)amino]propanoic acid;
(2S)-2-[(2-cyclohexylacetyl)amino]-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]-benzoyl}amino)-2-{[(E)-3-phenylprop-2-enoyl]amino}propanoic acid;
(2S)-2-[(2-chlorobenzoyl)amino]-3-({2-hydroxy-4-[2-(1,4,5,6-tetrahydropyrimidin-2-ylamino)ethoxy]benzoyl}amino)propanoic acid;
(2S)-3-({2-hydroxy-4-[2-(1,4,5,6-tetrahydropyrimidin-2-ylamino)ethoxy]benzoyl}-amino)-2-[(2-methylbenzoyl)amino]propanoic acid;
(2S)-3-({2-hydroxy-4-[2-(1,4,5,6-tetrahydropyrimidin-2-ylamino)ethoxy]benzoyl}-amino)-2-[(2-methoxybenzoyl)amino]propanoic acid;
(2S)-2-[(4-chlorobenzoyl)amino]-3-({2-hydroxy-4-[2-(1,4,5,6-tetrahydropyrimidin-2-ylamino)ethoxy]benzoyl}amino)propanoic acid; A
(2S)-3-({2-hydroxy-4-[2-(1,4,5,6-tetrahydropyrimidin-2-ylamino)ethoxy]benzoyl}-amino)-2-[(4-methylbenzoyl)amino]propanoic acid;
(2S)-3-({2-hydroxy-4-[2-(1,4,5,6-tetrahydropyrimidin-2-ylamino)ethoxy]benzoyl}-amino)-2-[(4-methoxybenzoyl)amino]propanoic acid;
(2S)-2-[(2,5-dimethyl-3-furoyl)amino]-3-({2-hydroxy-4-[2-(1,4,5,6-tetrahydropyrimidin-2-ylamino)ethoxy]benzoyl}amino)propanoic acid;
(2S)-2-[(2-bromobenzoyl)amino]-3-({2-hydroxy-4-[2-(1,4,5,6-tetrahydropyrimidin-2-ylamino)ethoxy]benzoyl}amino)propanoic acid;
(2S)-2-[(4-bromobenzoyl)amino]-3-({2-hydroxy-4-[2-(1,4,5,6-tetrahydropyrimindin-2-ylamino)ethoxy]benzoyl}amino)propanoic acid;
(2S)-2-[(2,30dimethylbenzoyl)amino]-3-({2-hydroxy-4-[2-(1,4,5,6-tetrahydropyrimidin-2-ylamino)ethoxy]benzoyl}amino)propanoic acid;
(2S)-2-[(3-chlorobenzoyl)amino]-3-({2-hydroxy-4-[2-(1,4,5,6-tetrahydropyrimidin-2-ylamino)ethoxy]benzoyl}amino)propanoic acid or pharmaceutical salts thereof.

16. A compound of claim 1 wherein G is pyrimidin-2-yl and R⁴ is NHCO₂R⁹.]

17. A compound of claim 1 which is:
(2S)-3-({2-hydroxy-4-[2-(pyrimidin-2-ylamino)ethoxy]benzoyl}amino)-2-[(phenoxycarbonyl)amino]propanoic acid;
(2S)-2-{[(benzyloxy}carbonyl]amino}-3-({2-hydroxy-4-[2-(pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-[(isobutoxycarbonyl)amino]propanoic acid;
(2S)-3-({2-hydroxy-4-[2-(pyrimidin-2-ylamino)ethoxy]benzoyl}amino)-2-{[(4-methoxyphenoxy)carbonyl]amino}propanoic acid;
(2S)-3-({2-hydroxy-4-[2-(pyrimidin-2-ylamino)ethoxy]benzoyl}amino)-2-{[(octyloxy)carbonyl]amino}propanoic acid;
(2S)-2-[(butoxycarbonyl)amino]-3-({2-hydroxy-4-[2-(pyrimidin-2-ylamino)ethoxy]-benzoyl}amino)propanoic acid;
(2S)-3-({2-hydroxy-4-[2-(pyrimidin-2-ylamino)ethoxy]benzoyl}amino)-2-{[(2,2,2-trichloroethoxy)carbonyl]amino}propanoic acid;
(2S)-3-({2-hydroxy-4-[2-(pyrimidin-2-ylamino)ethoxy]benzoyl}amino)-2-{[(neopentyloxy)carbonyl]amino}propanoic acid;
(2S)-3-({2-hydroxy-4-[2-(pyrimidin-2-ylamino)ethoxy]benzoyl}amino)-2-({[(4-nitrobenzyl)oxy]carbonyl}amino)propanoic acid;
(2S)-2{[(hexyloxy)carbonyl]amino}-3-({2-hydroxy-4-[2-(pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-{[(prop-2-ynyloxy)carbonyl]amino}propanoic acid;
(2S)-3-({2-hydroxy-4-[2-(pyrimidin-2-ylamino)ethoxy]benzoyl}amino)-2-{[(4-methylphenoxy)carbonyl]amino}propanoic acid;
(2S)-3-({2-hydroxy-4-[2-(pyrimidin-2-ylamino)ethoxy]benzoyl}amino)-2-[(methoxycarbonyl)amino]pronpanoic acid;
(2S)-2-[(ethoxycarbonyl)amino]-3-({2-hydroxy-4-[2-(pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-[(propoxycarbonyl)amino]propanoic acid;
(2S)-3-({2-hydroxy-4-[2-(pyrimidin-2-ylamino)ethoxy]benzoyl}amino)-2-[(isopropoxycarbonyl)amino]propanoic acid;
(2S)-2-{[(allyoxy)carbonyl]amino}-3-({2-hydroxy-4-[2-(pyrimidin-2-ylamino)ethoxy]benzoyl}amino)propanoic acid; or
(2S)-2-{[(but-3-enyloxy)carbonyl]amino}-3-({2-hydroxy-4-[2-pyrimidin-2-ylamino)ethoxy]benzoyl}amino)propanoic acid or pharmaceutical salts thereof.

18. A compound of claim 1 wherein G is pyrimidin-2-yl and R⁴ is NHCONH⁹.

19. A compound of claim 1 which is:
(2S)-2-[(anilinocarbonyl)amino]-3-({2-hydroxy-4-[2-(pyrimidin-2-ylamino)ethoxy]-benzoyl}amino)propanoic acid;
(2S)-2-{[tert-butylamino)carbonyl]amino}-3-({2-hydroxy-4-[2-(pyrimidin-2-ylamino)ethoxy]benzoyl}amino)propanoic acid;
(2S)-2-{[(butylamino)carbonyl]amino}-3-({2-hydroxy-4-[2-(pyrimidin-2-ylamino)-ethoxy]benzoyl}amino)propanoic acid;
(2S)-3-({2-hydroxy-4-[2-(pyrimidin-2-ylamino)ethoxy]benzoyl}amino)-2-{[(4-methoxyanilino)carbonyl]amino}propanoic acid;
(2S)-2-{[(2-ethylanilino)carbonyl]amino}-3-({2-hydroxy-4-[2-(pyrimidin-2-ylamino)ethoxy]benzoyl}amino)propanoic acid;
(2S)-2-{[(allylamino)carbonyl]amino}-3-({2-hydroxy-4-[2-(pyrimidin-2-ylamino)ethoxy]benzoyl}amino)propanoic acid;
(2S)-2-{[(2,4-dichloroanilino)carbonyl]amino}-3-({2-hydroxy-4-[2-pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-[(2-toluidinocarbonyl)amino]propanoic acid;
(2S)-3-({2-hydroxy-4-[2-(pyrimidin-2-ylamino)ethoxy]benzoyl}amino)-2-{[(2-methoxyanilino)carbonyl]amino}propanoic acid;
(2S)-2-{[(2-chloroanilino)carbonyl]amino}-3-({2-hydroxy-4-[2-(pyrimidin-2-ylamino)ethoxy]benzoyl}amino)propanoic acid;
(2S)-2-{[(2-bromoanilino)carbonyl]amino}-3-({2-hydroxy-4-[2-(pyrimidin-2-ylamino)ethoxy]benzoyl}amino)propanoic acid;
(2S)-2-{[([1,1′-biphenyl]-2-ylamino)carbonyl]amino}-3-({2-hydroxy-4-[2-(pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-[(4-toluidinocarbonyl)amino]propanoic acid;
(2S)-3-({2-hydroxy-4-[2-(pyrimidin-2-ylamino)ethoxy]benzoyl}amino)-2-({[4-trifluoromethyl)anilino]carbonyl}amino)propanoic acid;
(2S)-3-({2-hydroxy-4-[2-(pyrimidin-2-ylamino)ethoxy]benzoyl}amino)-2-({[4-(trifluoromethoxy)anilino]carbonyl}amino)propanoic acid;
(2S)-2-{[(4-chloroanilino)carbonyl]amino}-3-({2-hydroxy-4-[2-(pyrimidin-2-ylamino)ethoxy]benzoyl}amino)propanoic acid;
(2S)-2-{[(4-fluoroanilino)carbonyl]amino}-3-({2-hydroxy-4-[2-(pyrimidin-2-ylamino)ethoxy]benzoyl}amino)propanoic acid;
(2S)-2-{[(4-acetylanilino)carbonyl]amino}-3-({2-hydroxy-4-[2-(pyrimidin-2-ylamino)ethoxy]benzoyl}amino)propanoic acid;
(2S)-2-{[(cyclohexylamino)carbonyl]amino}-3-({2-hydroxy-4-[2-(pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-{[(1-naphthylamino)carbonyl]amino}propanoic acid;
(2S)-2-{[(benzylamino)carbonyl]amino}-3-({2-hydroxy-4-[2-(pyrimidin-2-ylamino)ethoxy]benzoyl}amino)propanoic acid;
(2S)-3-({2-hydroxy-4-[2-(pyrimidin-2-ylamino)ethoxy]benzoyl}amino)-2-([{[2-phenylethyl)amino]carbonyl}amino)propanoic acid; or
(2S)-3-({2-hydroxy-4-[2-(pyrimidin-2-ylamino)ethoxy]benzoyl}amino-2-{[(octylamino)carbonyl]amino}propanoic acid or pharmaceutical salts thereof.

20. A compound of claim 1 wherein G is 4,5-dihydro-1H-imidazolyl and R⁴ is NHCO₂R⁹.

21. A compound of claim 1 which is:
(2S)-2-{[(benzyloxy)carbonyl]amino}-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}-amino)-2-[(methoxycarbonyl)amino]propanoic acid;
(2S)-3-({4-[2-(4,5-dihydro-1H-imidazol-2-ylamino)ethoxy]-2-hydroxybenzoyl}-amino)-2-[(ethoxycarbonyl)amino]propanoic acid;
(2S)-3-({4-[2-(4,5-dihydro-1H-imidazol-2-ylamino)ethoxy]-2-hydoxybenzoyl}-amino)-2-[(propoxycarbonyl)amino]propanoic acid;
(2S)-3-({4-[2-(4,5-dihyrdo-1H-imidazol-2-ylamino)ethoxy]-2-hydroxybenzoyl}-amino)-2-[(isopropoxycarbonyl)amino]propanoic acid;
(2S)-2-{[(allyloxy)carbonyl]amino}-3-({4-[2-(4,5-dihydro-1H-imidazol-2-ylamino)ethoxy]-2-hydroxybenzoyl}amino)propanoic acid;
(2S)-2-{[(but-3-enyloxy)carbonyl]amino}-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}-amino)-2-{[(prop-2-ynloxy)carbonyl]amino}propanoic acid;
(2S)-3-({4-[2-(4,5-dihydro-1H-imidazol-2-ylamino)ethoxy]-2-hydroxybenzoyl}-amino)-2-{[(hexyloxy)carbonyl]amino}propanoic acid;
(2S)-3-({4-[2-(4,5-dihydro-1H-imidazol-2-ylamino)ethoxy]-2-hydroxybenzoyl}-amino)-2-{[(octyloxy)carbonyl]amino}propanoic acid;
(2S)-3-({4-[2-(4,5-dihydro-1H-imidazol-2-ylamino)ethoxy]-2-hydroxybenzoyl}amino)-2-{[(neopentyloxy)carbonyl]amino}propanoic acid;
(2S)-2-[(butoxycarbonyl)amino]-3-({4-[2-(4,5-dihydro-1H-imidazol-2-ylamino)ethoxy]-2-hydroxybenzoyl}amino)propanoic acid; or
(2S)-3-({4-[2-(4,5-dihydro-1H-imidazol-2-ylamino)ethoxy]-2-hydroxybenzoyl}amino)-2-[(isobutoxycarbonyl)amino]propanoic acid;
or a pharmaceutically acceptable salt form thereof.

22. A compound of claim 1 wherein G is 4,5-dihydro-1H-imidazolyl and R⁴ is NHCONHR⁹.

23. A compound of claim 1 which is:
(2S)-2-{[(butylamino)carbonyl]amino)-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}amino)-2-{[(hexylamino)carbonyl]amino}propanoic acid;
(2S)-3-({4-[2-(4,5-dihydro-1H-imidazol-2-ylamino)ethoxy]-2-hydroxybenzoyl}-amino)-2-{[(octylamino)carbonyl]amino}propanoic acid;
(2S)-2-{[(allylamino)carbonyl]amino}-3-({4-[2-(4,5-dihydro-1H-imidazol-2-ylamino)ethoxy]-2-hydroxybenzoyl}amino)propanoic acid;
(2S)-2-{[(cyclohexylamino)carbonyl]amino}-3-({4-[2-(4,5-dihydro-1H-imidazol-2-ylamino)ethoxy]-2-hydroxybenzoyl}amino)propanoic acid;
(2S)-2-{[(benzylamino)carbonyl]amino}-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}amino)-2-({[(1S,2R)-2-phenylcyclopropylamino]carbonyl}amino)propionic acid;
(2S)-3-({4-[2-(4,5-dihydro-1H-imidazol-2-ylamino)ethoxy]-2-hydroxybenzoyl}-amino)-2-{[(2-methoxyanilino)carbonyl]amino}propanoic acid;
(2S)-2-{[(1,1′-biphenyl]-2-ylamino)carbonyl]amino}-3-({4-[2-(4,5-dihydro-1H-imidazol-2-ylamino)ethoxy]-2-hydroxybenzoyl}amino)propanoic acid; or
(2S)-3-({4-[2-(4,5-dihydro-1H-imidazol-2-ylamino)ethoxy]-2-hydroxybenzoyl}-amino)-2-({[(2-phenylethyl)amino]carbonyl}amino)propanoic acid or pharmaceutical salts thereof.

24. A compound of claim 1 wherein G is 4,5-dihydro-1H-imidazol and R⁴ is NHCOR⁹.

25. A compound of claim 1 which is:
(2S)-3-({4-[2-(4,5-dihydro-1H-imidazol-2-ylamino)ethoxy]-2-hydroxybenzoyl}-amino)-2-(isobutyrylamino)propanoic acid;
(2S)-2-(butyrylamino)-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}-amino)-2-(hexanoylamino)propanoic acid;
(2S)-3-({4-[2-(4,5-dihydro-1H-imidazol-2-ylamino)ethoxy]-2-hydroxybenzoyl}-amino)-2-(pentanoylamino)propanoic acid;
(2S)-3-({4-[2-(4,5-dihydro-1H-imidazol-2-ylamino)ethoxy]-2-hydroxybenzoyl}-amino)-2-[(3,3-dimethylbutanoyl)amino]propanoic acid;
(2S)-3-({4-[2-(4,5-dihydro-1H-imidazol-2-ylamino)ethoxy]-2-hydroxybenzoyl}-amino)-2-{[(2,2,3,3-tetramethylcyclopropyl)carbonyl]amino}propanoic acid;
(2S)-2-{[2-(1-adamantyl)acetyl]amino}-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}-amino)-2-(pent-4-ynoylamino)propanoic acid;
(2S)-2-[(cyclohexylcarbonyl)amino]-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}-amino)-2-[(2-phenylacetyl)amino]propanoic acid;
(2S)-3-({4-[2-(4,5-dihydro-1H-imidazol-2-ylamino)ethoxy]-2-hydroxybenzoyl}-amino)-2-[(3-phenylpropanoyl)amino]propanoic acid;
(2S)-2-[(2-cyclohexylacetyl)amino]-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}-amino)-2-{[(E)-3-phenylprop-2-enoyl]amino)propanoic acid;
(2S)-2-[(2-chlorobenzoyl)amino]-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}amino)-2-[(2-methylbenzoyl)amino]propanoic acid;
(2S)-3-({4-[2-(4,5-dihydro-1H-imidazol-2-ylamino)ethoxy]-2-hydroxybenzoyl}amino)-2-[(2-methoxybenzoyl)amino]propanoic acid;
(2S)-2-[(4-chlorobenzoyl)amino]-3-({4-[2-(4,5-dihydro-1H-imidazol-2-ylamino)ethoxy]-2-hydroxybenzoyl}amino)propanoic acid;
(2S)-3-({4-[2-(4,5-dihydro-1H-imidazol-2-ylamino)ethoxy]-2-hydroxybenzoyl}amino)-2-[(4-methylbenzoyl)amino]propanoic acid;
(2S)-3-({4-[2-(4,5-dihydro-1H-imidazol-2-ylamino)ethoxy]-2-hydroxybenzoyl}amino)-2-[(4-methoxybenzoyl)amino]propanoic acid;
(2S)-3-({4-[2-(4,5-dihydro-1H-imidazol-2-ylamino)ethoxy]-2-hydroxybenzoyl}amino)-2-[(2,5-dimethyl-3-furoyl)amino]propanoic acid;
(2S)-2-[(2-bromobenzoyl)amino]-3-({4-[2-(4,5-dihydro-1H-imidazol-2-ylamino)ethoxy]-2-hydroxybenzoyl}amino)propanoic acid;
(2S)-2-[(4-bromobenzoyl)amino]-3-({4-[2-(4,5-dihydro-1H-imidazol-2-ylamino)ethoxy]-2-hydroxybenzoyl}amino)propanoic acid;
(2S)-3-({-[2-(4,5-dihydro-1H-imidazol-2-ylamino)ethoxy]-2-hydroxybenzoyl}-amino)-2-[(2,3-dimethylbenzoyl)amino]propanoic acid; or
(2S)-2[(3-chlorobenzoyl)amino]-3-({4-[2-(4,5-dihydro-1H-imidazol-2-ylamino)ethoxy]-2-hydroxybenzoyl}amino)propanoic acid or pharmaceutical salts thereof.

26. A compound of claim 1 wherein G is 3,4,5,6-tetrahydro-2H-azepinyl and R⁴ is NHCO₂R⁹.

27. A compound of claim 1 which is:
(2S)-2-{[(benzyloxy)carbonyl]amino}-3-({2-hydroxy-4-[2-(3,4,5,6-tetrahydro-2H-azepin-7-ylamino)ethoxy]benzoyl}amino)propanoic acid;
(2S)-3-({2-hydroxy-4-[2-(3,4,5,6-tetrahydro-2H-azepin-7-ylamino)ethoxy]benzoyl}amino)-2-[(methoxycarbonyl)amino]propanoic acid;
(2S)-2-[(ethoxycarbonyl)amino]-3-({2-hydroxy-4-[2-(3,4,5,6-tetrahydro-2H-azepin-7-ylamino)ethoxy]benzoyl}amino)propanoic acid;
(2S)-3-({2-hydroxy-4-[2-(3,4,5,6-tetrahydro-2H-azepin-7-ylamino)ethoxy]benzoyl}amino-2-[(propoxycarbonyl)amino]propanoic acid;
(2S)-3-({hydroxy-4-[2-(3,4,5,6-tetrahydro-2H-azepin-7-ylamino)ethoxy]benzoyl}amino)-2-[(isopropoxycarbonyl)amino]propanoic acid;
(2S)-2-{[(allyloxy)carbonyl]amino}-3-({2-hydroxy-4-[2-(3,4,5,6-tetrahydro-2H-azepin-7-ylamino)ethoxy]benzoyl}amino)propanoic acid;
(2S)-2-{[(but-3-enyloxy)carbonyl]amino}-3-({2-hydroxy-4-[2-(3,4,5,6-tetrahydro-2H-azepin-7-ylamino)ethoxy]benzoyl}amino)propanoic acid;
(2S)-3-({2-hydroxy-4-[2-(3,4,5,6-tetrahydro-2H-azepin-7-ylamino)ethoxy]benzoyl}amino)-2-{[(prop-2-ynyloxy)carbonyl]amino}propanoic acid;
(2S)-2-{[(hexyloxy)carbonyl]amino}-3-({2-hydroxy-4-[2-(3,4,5,6-tetrahydro-2H-azepin-7-ylamino)ethoxy]benzoyl}amino)propanoic acid;
(2S)-3-({2-hydroxy-4-[2-(3,4,5,6-tetrahydro-2H-azepin-7-ylamino)ethoxy]benzoyl}amino)-2-{[(octyloxy)carbonyl]amino}propanoic acid;
(2S)-3-({2-hydroxy-4-[2-(3,4,5,6-tetrahydro-2H-azepin-7-ylamino)ethoxy]benzoyl}amino-2-{[(neopentyloxy)carbonyl]amino}propanoic acid;
(2S)-3-({2-hydroxy-4-[2-(3,4,5,6-tetrahydro-2H-azepin-7-ylamino)ethoxy]benzoyl}-amino-2-{[(2,2,2-trichloroethoxy)carbonyl]amino}propanoic acid;
(2S)-2-[(butoxycarbonyl)amino]-3-({2-hydroxy-4-[2-(3,4,5,6-tetrahydro-2H-azepin-7-ylamino)ethoxy]benzoyl}amino)propanoic acid; or
(2S)-3-({2-hydroxy-4-[2-(3,4,5,6-tetrahydro-2H-azepin-7-ylamino)ethoxy]benzoyl}amino)-2-[(isobutoxycarbonyl)amino]propanoic acid or pharmaceutical salts thereof.

28. A compound of claim 1 wherein G is NH₂C(NH)— and R⁴ is NHCO₂R⁹.

29. A compound of claim 1 which is:
(2S)-3-{[4-(2-{[amino(imino)methyl]amino}ethoxy)-2-hydroxybenzoyl]amino}-2-{[(benzyloxy)carbonyl]amino}propanoic acid; 140
(2S)-3-{[4-(2-{[amino(imino)methyl]amino}ethoxy)-2-hydroxybenzoyl]amino}-2-[(methoxycarbonyl)amino]propanoic acid;
(2S)-3-{[4-(2-{[amino(imino)methyl]amino}ethoxy)-2-hydroxybenzoyl]amino}-2-[(ethoxycarbonyl)amino]propanoic acid;
(2S)-3-{[4-(2-{[amino(imino)methyl]amino}ethoxy)-2-hydroxybenzoyl]amino}-2-[(propoxycarbonyl)amino]propanoic acid;
(2S)-3-{[4-(2-{[amino(imino)methyl]amino}ethoxy)-2-hydroxybenzoyl]amino}-2-[(isopropoxycarbonyl)amino]propanoic acid;
(2S)-2-{[(allyloxy)carbonyl]amino}-3-{[4-(2-{[amino(imino)methyl]amino}ethoxy)-2-hydroxybenzoyl]amino}propanoic acid;
(2S)-3-{[4-(2-{[amino(imino)methyl]amino}ethoxy-2-hydroxybenzoyl]amino}-2-{[(but-3-enyloxy)carbonyl]amino}propanoic acid;
(2S)-3-{[4-(2-{[amino(imino)methyl]amino}ethoxy)-2-hydoxybenzoyl]amino}-2-{[(butoxycarbonyl)amino]propanoic acid;
(2S)-3-{4-(2-{[amino(imino)methyl]amino}ethoxy)-2-hydroxybenzoyl]amino}-2-{[(2,2,2-trichloroethoxy)carbonyl]amino}propanoic acid;
(2S)-3-{[4-(2-{[amino(imino)methyl]amino}ethoxy)-2-hydroxybenzoyl]amino}-2-{[(neopentyloxy)carbonyl]amino}propanoic acid;
(2S)-3-{[4-(2-{[amino(imino)methyl]amino}ethoxy)-2-hydroxybenzoyl]amino}-2-{[(hexyloxy)carbonyl]amino}propanoic acid;
(2S)-3-{[4-(2-{[amino(imino)methyl]amino}ethoxy)-2-hydroxybenzoyl]amino}-2-{[(prop-2-ynyloxy}carbonyl]amino}propanoic acid;
(2S)-3-{[4-(2-{[amino(imino)methyl]amino}ethoxy)-2-hydroxybenzoyl]amino}-2-{[([1,1′-biphenyl]-2-ylmethoxy)carbonyl]amino}propanoic acid;
(2S)-3-{[4-(2-{[amino(imino)methyl]amino}ethoxy)-2-hydroxybenzoyl]amino}-2-({[(4-bromobenzyl)oxy]carbonyl}amino)propanoic acid;
(2S)-3-{[4-(2-{[amino(imino)methyl]amino}ethoxy)-2-hydroxybenzoyl]amino}-2-({[(4-fluorobenzyl)oxy]carbonyl}amino)propanoic acid;
(2S)-3-{[4-(2-{[amino(imino)methyl]amino}ethoxy)-2-hydroxybenzoyl]amino}-2-({[(2-bromobenzyl)oxy]carbonyl}amino)propanoic acid; or
(2S)-3-{[4-(2-{[amino(imino)methyl]amino}ethoxy)-2-hydroxybenzoyl]amino}-2-[({[4-(trifluoromethyl)benzyl]oxy}carbonyl)amino]propanoic acid or pharmaceutical salts thereof.

30. A compound of claim 1 wherein G in NH₂C(═NH)— and R⁴ is NHCONHR⁹.

31. A compound of claim 1 which is:
(2S)-3-{[4-(2-{[4-(2-{[amino(imino)methyl]amino}ethoxy)-2-hydroxybenzoyl]amino}-2-[(2-toluidinocarbonyl)amino]propanoic acid;
(2S)-3-{[4-(2-{[amino(imino)methyl]amino}ethoxy)-2-hydroxybenzoyl]amino}-2-{[2-methoxyanilino)carbonyl]amino}propanoic acid;
(2S)-3-{[4-(2-{[amino(imino)methyl]amino}ethoxy)-2-hydroxybenzoyl]amino}-2-{[(2-chloroanilino)carbonyl]amino}propanoic acid;
(2S)-3-{[4-(2-{[amino(imino)methyl]amino}ethoxy)-2-hydroxybenzoyl]amino}-2-{[(2-bromoanilino)carbonyl]amino}propanoic acid;
(2S)-3-{[4-(2-{[amino(imino)methyl]amino}ethoxy)-2-hydroxybenzoyl]amino}-2-{[([1,1′-biphenyl]-2-ylamino)carbonyl]amino}propanoic acid;
(2S)-3-{[4-(2-{[amino(imino)methyl]amino}ethoxy)-2-hydroxybenzoyl]amino}-2-[(4-toluidinocarbonyl)amino]propanoic acid;
(2S)-3-{[4-(2-{[amino(imino)methyl]amino}ethoxy)-2-hydroxybenzoyl]amino}-2-({[4-(trifluoromethoxy)anilino]carbonyl}amino)propanoic acid;
(2S)-3-{[4-(2-{[amino(imino)methyl]amino}ethoxy)-2-hydroxybenzoyl]amino}-2-{[(4-chloroanilino)carbonyl]amino}propanoic acid;
(2S)-3-{[4-(2-{[amino(imino)methyl]amino}ethoxy)-2-hydroxybenzoyl]amino}-2-{[(4-fluoroanilino)carbonyl]amino}propanoic acid;
(2S)-2-{[(4-acetylanilino)carbonyl]amino}-3-{[4-(2-{[amino(imino)methyl]-amino}ethoxy)-2-hydroxybenzoyl]amino}propanoic acid;
(2S)-3-{[4-(2-{[amino(imino)methyl]amino}ethoxy)-2-hydroxybenzoyl]amino}-2-{[(cyclohexylamino)carbonyl]amino}propanoic acid;
(2S)-3-{[4-(2-{[amino(imino)methyl]amino}ethoxy)-2-hydroxybenzoyl]amino}-2-{[(1-naphthylamino)carbonyl]amino}propanoic acid;
(2S)-3-{[4-(2-{[amino(imino)methyl]amino}ethoxy)-2-hydroxybenzoyl]amino}-2-{[(benzoylamino)carbonyl]amino}propanoic acid;
(2S)-3-{[4-(2-{[amino(imino)methyl]amino}ethoxy)-2-hydroxybenzoyl]amino}-2-({[(2-phenylethyl)amino]carbonyl}amino)propanoic acid;
(2S)-3-{[4-(2-{[amino(imino)methyl]amino}ethoxy)-2-hydroxybenzoyl]amino}-2-{[(octylamino)carbonyl]amino}propanoic acid;
(2S)-3-{[4-(2-{[amino(imino)methyl]amino}ethoxy)-2-hydroxybenzoyl]amino}-2-{[(4-methoxyanilino)carbonyl]amino}propanoic acid; or
(2S)-3-{[4-(2-{[amino(imino)methyl]amino}ethoxy)-2-hydroxybenzoyl]amino}-2-[(anilinocarbonyl)amino]propanoic acid or pharmaceutical salts thereof.

32. A compound of claim 1 wherein G is NH₂C(═NH)— and R⁴ is NHCOR⁹.

33. A compound of claim 1 which is:
(2S)-3-{[4-(2-{[amino(imino)methyl]amino}ethoxy)-2-hydroxybenzoyl]-amino}-2-(isobutyrylamino)propanoic acid;
(2S)-3-{[4-(2-{[amino(imino)methyl]amino}ethoxy)-2-hydroxybenzoyl]amino}-2-(butyrylamino)propanoic acid;
(2S)-3-{[4-(2-{[amino(imino)methyl]amino}ethoxy-2-hydroxybenzoyl]amino}-2-(hexanoylamino)propanoic acid;
(2S)-3-{[4-(2-{[amino(imino)methyl]amino}ethoxy)-2-hydroxybenzoyl]amino}-2-(pentanoylamino)propanoic acid;
(2S)-3-{[4-(2-{[amino(imino)methyl]amino}ethoxy)-2-hydroxybenzoyl]amino}-2-[(3,3-dimethylbutanoyl)amino]propanoic acid;
(2S)-3-{[4-(2-{[amino(imino)methyl]amino}ethoxy)-2-hydroxybenzoyl]amino}-2-{[(2,2,3,3-tetramethylcyclopropyl)carbonyl]amino}propanoic acid;
(2S)-2-{[2-(1-adamantyl)acetyl]amino}-3-{[4-(2-{[amino(imino)methyl]amino}-ethoxy)-2-hydroxybenzoyl]amino}propanoic acid;
(2S)-3-{[4-(2-{[amino(imino)methyl]amino}ethoxy)-2-hydroxybenzoyl]amino}-2-(pent-4-ynoylamino)propanoic acid;
(2S)-3-{[4-(2-{[amino(imino)methyl]amino}ethoxy)-2-hydroxybenzoyl]amino}-2-[(cyclohexylcarbonyl)amino]propanoic acid;
(2S)-3-{[4-(2-{[amino(imino)methyl]amino}ethoxy)-2-hydroxybenzoyl]amino-2-[(2-phenylacetyl)amino]propanoic acid;
(2S)-3-{[4-(2-{[amino(imino)methyl]amino}ethoxy)-2-hydroxybenzoyl]amino}-2-[(3-phenylpropanoyl)amino]propanoic acid;
(2S)-3-{[4-(2-{[amino(imino)methyl]amino}ethoxy)-2-hydroxybenzoyl]amino}-2-[(2-cyclohexylacetyl)amino]propanoic acid;
(2S)-3-{[4-(2-{[amino(imino)methyl]amino}ethoxy)-2-hydroxybenzoyl]amino}-2-{[(E)-3-phenylprop-2-enoyl]amino}propanoic acid;
(2S)-3-{[4-(2-{[amino(imino)methyl]amino}ethoxy)-2-hydroxybenzoyl]amino}-2-[(2-chlorobenzoyl)amino]propanoic acid;
(2S)-3-{[4-(2-{[amino(imino)methyl]amino}ethoxy)-2-hydroxybenzoyl]amino}-2-[(2-methylbenzoyl)amino]propanoic acid;
(2S)-3-{[4-(2-{[amino(imino)methyl]amino}ethoxy)-2-hydroxybenzoyl]amino}-2-[(2-methoxybenzoyl)amino]propanoic acid;
(2S)-3-{[4-(2-{[amino(imino)methyl]amino}ethoxy)-2-hydroxybenzoyl]amino}-2-[(4-chlorobenzoyl)amino]propanoic acid;
(2S)-3-{[4-(2-{[amino(imino)methyl]amino}ethoxy)-2-hydroxybenzoyl]amino}-2-[(4-methylbenzoyl)amino]propanoic acid;
(2S)-3-{[4-(2-{[amino(imino)methyl]amino}ethoxy)-2-hydroxybenzoyl]amino}-2-[(4-methoxybenzoyl)amino]propanoic acid;
(2S)-3-{[4-(2-{[amino(imino)methyl]amino}ethoxy)-2-hydroxybenzoyl]amino}-2-[(pyridin-3ylcarbonyl)amino]propanoic acid;
(2S)-3-{[4-(2-{[amino(imino)methyl]amino}ethoxy)-2-hydroxybenzoyl]amino}-2-(isonicotinoylamino)propanoic acid;
(2S)-3-{[4-(2-{[amino(imino)methyl]amino}ethoxy)-2-hydroxybenzoyl]amino}-2-[(2,5-dimethyl-3-furoyl)amino]propanoic acid;
(2S)-3-{[4-(2-{[amino(imino)methyl]amino}ethoxy)-2-hydroxybenzoyl]amino}-2-[(2-bromobenzoyl)amino]propanoic acid;
(2S)-3-{[4-(2-{[amino(imino)methyl]amino}ethoxy)-2-hydroxybenzoyl]amino}-2-[(4-bromobenzoyl)amino]propanoic acid;
(2S)-3-{[4-(2-{[amino(imino)methyl]amino}ethoxy)-2-hydroxybenzoyl]amino}-2-[(2,3-dimethylbenzoyl)amino]propanoic acid;
(2S)-3-{[4-(2-{[amino(imino)methyl]amino}ethoxy)-2-hydroxybenzoyl]amino}-2-[(3-chlorobenzoyl)amino]propanoic acid;
(2S)-3-{[4-(2-{[amino(imino)methyl]amino}ethoxy)-2-hydroxybenzoyl]amino}-2-(benzoylamino)propanoic acid;
(2S)-3-{[4-(2-{[amino(imino)methyl]amino}ethoxy)-2-hydroxybenzoyl]amino}-2-[(4-ethylbenzoyl)amino]propanoic acid; or
(2S)-3-{[4-(2-{[amino(imino)methyl]amino}ethoxy)-2-hydroxybenzoyl]amino}-2-[(4-butoxybenzoyl)amino]propanoic acid or pharmaceutical salts thereof.

34. A compound of claim 1 wherein G is R⁸NHCO—. R⁸ is benzyl and R⁴ is NHCO₂R⁹.

35. A compound of claim 1 which is:
(2S)-3-{[4-(2-{[(benzylamino)carbonyl]amino}ethoxy)-2-hydroxybenzoyl]amino}-2-{[(benzyloxycarbonyl]amino}propanoic acid;
(2S)-3-{[4-(2-{[(benzylamino)carbonyl]amino}ethoxy)-2-hydroxybenzoyl]amino}-2-{[(methoxycarbonyl]amino}propanoic acid;
(2S)-3-{[4-(2-{[(benzylamino)carbonyl]amino}ethoxy)-2-hydroxybenzoyl]amino}-2-{[(ethoxycarbonyl]amino}propanoic acid;
(2S)-3-{[4-(2-{[(benzylamino)carbonyl]amino}ethoxy)-2-hydroxybenzoyl]amino}-2-{[(propoxycarbonyl]amino}propanoic acid;
(2S)-3-{[4-(2-{[(benzylamino)carbonyl]amino}ethoxy)-2-hydroxybenzoyl]amino}-2-{[(isopropoxycarbonyl]amino}propanoic acid;
(2S)-2-{[(allyloxy)carbonyl]amino}3-3-[4-(2-{[benzylamino)carbonyl]amino}ethoxy)-2-hydroxybenzoyl]amino}propanoic acid;
(2S)-3-{[4-(2-{[(benzylamino)carbonyl]amino}ethoxy)-2-hydroxybenzoyl]amino}-2-{[(but-3-enyloxy)carbonyl]amino}propanoic acid;
(2S)-3-{[4-(2-{[(benzylamino)carbonyl]amino}ethoxy)-2-hydroxybenzoyl]amino}-2-{[(hexyloxy)carbonyl]amino}propanoic acid;
(2S)-3-{[4-(2-{[(benzylamino)carbonyl]amino}ethoxy)-2-hydroxybenzoyl]amino}-2-{[(octyloxy)carbonyl]amino}propanoic acid;
(2S)-3-{[4-(2-{[(benzylamino)carbonyl]amino}ethoxy)-2-hydroxybenzoyl]amino}-2-{[(neopentyloxy)carbonyl]amino}propanoic acid;
(2S)-3-{[4-(2-{[(benzylamino)carbonyl]amino}ethoxy)-2-hydroxybenzoyl]amino}-2-{[(2,2,2-trichloroethoxy)carbonyl]amino}propanoic acid;
(2S)-3-{[4-(2-{[(benzylamino)carbonyl]amino}ethoxy)-2-hydroxybenzoyl]amino}-2-[(butoxycarbonyl)amino]propanoic acid;
(2S)-3-{[4-(2-{[(benzylamino)carbonyl]amino}ethoxy)-2-hydroxybenzoyl]amino}-2-[(isobutoxycarbonyl)amino]propanoic acid or pharmaceutical salts thereof.

36. A compound of claim 1 wherein G is R⁸NHCO, R⁸ is pyridin-3yl methyl, and R⁴ is NHCO₂R⁹.

37. A compound of claim 1 which is:
(2S)-2-{[(benzyloxy)carbonyl]amino}-3-({2-hydroxy-4-[2-({[(pyridin-3-ylmethyl)amino]carbonyl]amino)ethoxy]benzoyl}amino)propanoic acid;
(2S)-3-({2-hydroxy-4-[2-({[pyridin-3-ylmethyl)amino]carbonyl}amino)-ethoxy]benzoyl}amino)-2-[(methoxycarbonyl)amino]propanoic acid;
(2S)-2-[(ethoxycarbonyl)amino]-3-({2-hydroxy-4-[2-({[pyridin-3-ylmethyl)-amino]carbonyl}amino)ethoxy]benzoyl}amino)propanoic acid;
(2S)-3-({2-hydroxy-4-[2-({[pyridin-3-ylmethyl)amino]carbonyl}amino)ethoxy]-benzoyl}amino)-2-[(propoxycarbonyl)amino]propanoic acid;
(2S)-3-({2-hydroxy-4-[2-({[pyridin-3-ylmethyl)amino]carbonyl}amino)ethoxy]-benzoyl}amino)-2-[(isopropoxycarbonyl)amino]propanoic acid;
(2S)-2-{[l(allyloxy)carbonyl]amino}-3-({2-hydroxy-4-[2-({[(pyridin-3-ylmethyl)amino]carbonyl}amino)ethoxy]benzoyl}amino)propanoic acid;
(2S)-2-{[(but-3-enyloxy)carbonyl]amino}-3-({2-hydroxy-4-[2-{[(pyridin-3-ylmethyl)amino]carbonyl}amino)ethoxy]benzoyl}amino)propanoic acid;
(2S)-3-({2-hydroxy-4-[2-({[(pyridin-3-ylmethyl)amino]carbonyl}amino)-ethoxy]benzoyl}amino)-2-{[(prop-2-ynyloxy)carbonyl]amino}propanoic acid;
(2S)-2-{[(hexyloxy)carbonyl]amino}-3-({2-hydroxy-4-[({[pyridin-3-ylmethyl)amino]carbonyl}amino)ethoxy]benzoyl}amino)propanoic acid;
(2S)-3-({2-hydroxy-4-[2-({[(pyridin-3-ylmethyl)amino]carbonyl}amino)ethoxy]-benzoyl}amino)-2-{[(octyloxy)carbonyl]amino}propanoic acid;
(2S)-3-({2-hydroxy-4-[2-({[(pyridin-3-ylmethyl)amino]carbonyl}amino)ethoxy]-benzoyl}amino)-2-{[(neopentyloxy)carbonyl]amino}propanoic acid;
(2S)-3-({2-hydroxy-4-[2-({[(pyridin-3-ylmethyl)amino]carbonyl}amino)ethoxy]-benzoyl}amino)-2-{[(2,2,2-trichloroethoxy)carbonyl]amino}propanoic acid;
(2S)-2-[(butoxycarbonyl)amino]-3-({2-hydroxy-4-[2-({[pyridin-3-ylmethyl)amino]-carbonyl}amino)ethoxy]benzoyl}amino)propanoic acid;
(2S)-3-({2-hydroxy-4-[2-({[(pyridin-3-ylmethyl)amino]carbonyl}amino)ethoxy]-benzoyl}amino)-2-[(isobutoxycarbonyl)amino]propanoic acid or pharmaceutical salts thereof.

38. A compound of claim 1 wherein G is R⁸NHCO and R⁸ is pyridin-4yl and R⁴ is NHCO₂R⁹.

39. A compound of claim 1 which is:
(2S)-2-[(benzyloxy)carbonyl]amino}-3-({2-hydroxy-4-[2-({[(pyridin-4-ylmethyl)amino]carbonyl}amino)ethoxy]benzoyl}amino)propanoic acid;
(2S)-3-({2-hydroxy-4-[2-({[(pyridin-4-ylmethyl)amino]carbonyl}amino)ethoxy]-benzoyl}amino)-2-[(methoxycarbonyl)amino]propanoic acid;
(2S)-2-[(ethoxycarbonyl)amino]-3-({2-hydroxy-4-[2-({[(pyridin-4-ylmethyl)-amino]carbonyl}amino)ethoxy]benzoyl}amino)propanoic acid;
(2S)-3-({2-hydroxy-4-[2-({[(pyridin-4-ylmethyl)amino]carbonyl}amino)ethoxy]-benzoyl}amino)-2-[(propoxycarbonyl)amino]propanoic acid;
(2S)-3-({2-hydroxy-4-[2-({[(pyridin-4-ylmethyl)amino]carbonyl}amino)ethoxy]-benzoyl}amino)-2-[(isopropoxycarbonyl)amino]propanoic acid;
(2S)-2-{[(allyloxy)carbonyl]amino}-3-({2-hydroxy-4-[2-({[(pyridin-4-ylmethyl)amino]carbonyl}amino)ethoxy:benzoyl}amino)propanoic acid;
(2S)-2-{[(but-3-enyloxy)carbonyl]amino}-3-({2-hydroxy-4-[2-({[(pyridin-4-ylmethyl)-amino]carbonyl}amino)ethoxy]benzoyl}amino)propanoic acid;
(2S)-3-({2-hydroxy-4-[2-({[(pyridin-4-ylmethyl)amino]carbonyl}amino)ethoxy]-benzoyl}amino)-2-{[(prop-2-ynyloxy)carbonyl]amino}propanoic acid;
(2S)-2-{[(hexyloxy)carbonyl]amino}-3-({2-hydroxy-4-{2-([{(pyridin-4-ylmethyl)-amino]carbonyl}amino)ethoxy]benzoyl}amino)propanoic acid;
(2S)-3-({2-hydroxy-4-[2-({[(pyridin-4-ylmethyl)amino]carbonyl}amino)ethoxy]-benzoyl}amino)-2-{[(octyloxy)carbonyl]amino}propanoic acid;
(2S)-3-({2-hydroxy-4-[2-({[(pyridin-4-ylmethyl)amino]carbonyl}amino)ethoxy]-benzoyl}amino)-2-{[(neopentyloxy)carbonyl]amino}propanoic acid;
(2S)-3-({2-hydroxy-4-[2-({[(pyridin-4-ylmethyl)amino]carbonyl}amino)ethoxy]-benzoyl}amino)-2-{[(2,2,2-trichloroethoxy)carbonyl]amino}propanoic acid;
(2S)-2-[(butoxycarbonyl)amino]-3-({2-hydroxy-4-[2-({[(pyridin-4-ylmethyl)amino]-carbonyl}amino)ethoxy]benzoyl}amino)propanoic acid; or
(2S)-3-({hydroxy-4-[2-({[(pyridin-4-ylmethyl)amino]carbonyl}amino)ethoxy]-benzoyl}amino)-2-[(isobutoxycarbonyl)amino]propanoic acid or pharmaceutical salts thereof.

40. A compound of claim 1 which is:
(2S)-2-{[(benzyloxy)carbonyl]amino}-3-({2-hydroxy-4-[2-({[(4-methylbenzyl)-amino]carbonyl}amino)ethoxy]benzoyl}amino)propanoic acid;
(2S)-2-{[(benzyloxy)carbonyl]amino}-3-({2-hydroxy-4-[2-({[(4-methoxybenzyl)-amino]carbonyl}amino)ethoxy]benzoyl}amino)propanoic acid;
(2S)-2-{[(benzyloxy)carbonyl]amino}-3-({4-[2-({[(4-chlorobenzyl)amino]-carbonyl}amino)ethoxy]-2-hydroxybenzoyl}amino)propanoic acid;
(2S)-2-{[(benzyloxy)carbonyl]amino}-3-[(4-{2-[({[4-(dimethylamino)benzyl]-amino}carbonyl)amino]ethoxy}-2-hydroxybenzoyl)amino]propanoic acid;
(2S)-2-{[(benzyloxy)carbonyl]amino}-3-[(2-hydroxy-4-{2-[({[4-trifluoromethoxy)-benzyl]amino}carbonyl)amino]ethoxy}benzoyl)amino]propanoic acid;
(2S)-2-{[(benzyloxy)carbonyl]amino}-3-({4-[2-({[(2-chlorobenzyl)amino]carbonyl}-amino)ethoxy]-2-hydroxybenzoyl}amino)propanoic acid;
(2S)-2-{[(benzyloxy)carbonyl]amino}-3-({2-hydroxy-4-[2-({[(2-methylbenzyl)-amino]carbonyl}amino)ethoxy]benzoyl}amino)propanoic acid;
(2S)-2-{[(benzyloxy)carbonyl]amino}-3-({4-[2-({[(2-bromobenzyl)amino]-carbonyl}amino)ethoxy]020hydroxybenzoyl}amino)propanoic acid;
(2S)-2-{[(benzyloxy)carbonyl]amino}-3-({4-[2-({[(2,4-dichlorobenzyl)amino]-carbonyl}amino)ethoxy]-2-hydroxybenzoyl}amino)propanoic acid;
(2S)-3-({4-[2-({[(2-aminobenzyl)amino]carbonyl}amino)ethoxy]-2-hydroxybenzoyl}-amino)-2-{[(benzyloxy)carbonyl]amino}propanoic acid; or
(2S)-2-{[(benzyloxy)carbonyl]amino}-3-({2-hydroxy-4-[2-({[(pyridin-2-ylmethyl)amino]carbonyl}amino)ethoxy]benzoyl}amino)propanoic acid or pharmaceutical salts thereof.

41. A compound of claim 1 which is:
(2S)-2-Benzenesulfonylamino-3-(2-hydroxy-4-[3-(1,4,5,6-tetrahydropyrimidin-2-ylamino)propoxy]benzoylamino)-propionic acid;
(2S)-2-Benzenesulfonylamino-3-{2-hydroxy-4-[2-(1,4,5,6-tetrahydropyrimidin-2-ylamino)ethoxy]benzoylamino}propionic acid tert-butyl ester;
(2S)-2-Benzenesulfonylamino-3-{2-hydroxy-5-[4-(pyrimidin-2-ylamino)-butoxy]-benzoylamino}propionic acid;
(2S)-2-Benzenesulfonylamino-3-{2-hydroxy-5-[4-(1,4,5,6-tetrahydropyrimidin-2-ylamino)butoxy]benzoylamino}propionic acid ethyl ester;
(2S)-2-Benzenesulfonylamino-3-{2-hydroxy-4-[3-(pyrimidin-2-ylamino)propoxy]-benzoylamino}propionic acid;
3-{2-Hydroxy-5-[3-(pyrimidin-2-ylamino)propoxy]benzoylamino}-3-phenyl-propionic acid;
(2S)-2-{(Adamantan-1-yloxycarbonylamino)-3-(2-hydroxy-4-[2-(1,4,5,6-tetrahydro-pyrimidin-2-ylamino)-ethoxy]-benzoylamino)}-propionic acid;
(2S)-2-Benzenesulfonylamino-3-(2-hydroxy-4-[3-(1,4,5,6-tetrahydropyrimidin-2-ylamino)propoxy]benzoylamino)-propionic acid ethyl ester;
3-{2-Hydroxy-5-[3-(pyrimidin-2-ylamino)-propxy)]-benzoylamino}-3-phenyl-propionic acid ethyl ester;
(2S)-2-(Adamantan-1-ylmethoxycarbonylamino)-3-{2-hydroxy-4-[2-(1,4,5,6-tetrahydropyrimidin-2-ylamino)ethoxy]benzoylamino}propionic acid;
(2S)-2-Benzenesulfonylamino-3-{2-hydroxy-4-[2-(1,4,5,6-tetrahydropyrimidin-2-ylamino)ethoxy]benzoylamino}propionic acid isopropyl ester;
(2S)-2-tert-Butoxycarbonylamino-3-{2-hydroxy-4-[2-(1,4,5,6-tetrahydropyrimidin-2-ylamino)ethoxylbenzoylamino}propionic acid; or
(2S)-2-Benzenesulfonyamino-3-{2-hydroxy-5-[4-(1,4,5,6-tetrahydropyrimidin-2-ylamino)butoxy]benzoylamino}propionic acid or pharmaceutical salts thereof.

42. A compound of claim 1 which is:
2(S)-Benzenesulfonylamino-3-[2-hydroxy-4-[(2-pyrimidin-2-ylamino)ethoxy]-benzoylamino]propionic acid ethyl ester;
2(S)-Benezenesulfonylamino-3-[2-hydroxy-4-[(2-pyrimidin-2-ylamino)ethoxy]-benzoylamino]propionic acid;
2(S)-Benzenesulfonylamino-3-[2-hydroxy-4-[2-(3,4,5,6-tetrahydropyrimidin-2-ylamino)ethoxy]benzoyl]amino]propionic acid hydrochloride;
2(S)-Benzenesulfonylamino-3-[2-hydroxy-4-[(2-pyrimidin-2ylamino)ethoxy]-benzoylamino]propionic acid ethyl ester hydrochloride;
2(S)-Benzyloxycarbonylamino-3-[2-hydroxy-4-[2-(3,4,5,6-tetrahydropyrimidin-2-ylamino)ethoxy]benzoylamino]propionic acid ethyl ester hydrochloride;
2(S)-Benzyloxycarbonylamino-3-[2-hydroxy-4-[2-(3,4,5,6-tetrahydropyrimidin-2-ylamino)ethoxy]benzoylamino]propionic acid hydrochloride;
3-[4-(2-Guanidineoethoxy)-2-hydroxy-benzoylamino]-3-phenylpropanoic acid ethyl ester hydrochloride;
3-[4-Guanidinoethoxy)-2-hydroxy-benzoylamino]-3-phenylpropanoic acid hydrochloride;
3-[2-hydroxy-4-[2-(pyrimidin-2-ylamino)ethoxy]benzoylamino]-3-pyridin-3-yl-propanoic acid ethyl ester;
3-[2-hydroxy-4-[2-(pyrimidin-2-ylamino)ethoxy]benzoylamino]-3-pyridin-3-yl-propanoic acid;
3-[2-hydroxy-4-[2-(3,4,5,6-tetrahydropyrimidin-2-ylamino)ethoxy]benzoylamino]-3-pyridin-3-ylpropanoic acid ethyl ester dihydrochloride;
3-[2-hydroxy-4-[2-(3,4,5,6-tetrahydropyrimidin-2-ylamino)ethoxy]benzoylamino]-3-pyridin-3-yl-propanoic acid;
3-[4-(2-Guanidino-ethoxy)-2-hydroxybenzoylamino -3-pyridin-3-yl-propanoic acid ethyl ester dihydrochloride;
3-[4-(2-Guanidino-ethoxy)-2-hydroxybenzoylamino]-3-pyridin-3-yl-propanoic acid;
3-[2-hydroxy-4-[2-(pyrimidin-2-ylamino)ethoxy]benzoylamino]-3-phenyl-propanoic acid ethyl ester;
3-[2-hydroxy-4-[2-(pyrimidin-2-ylamino)ethoxy]benzoylamino]-3-phenyl-propanoic acid hydrochloride;
3-[2-hydroxy-4-[2-(3,4,5,6-tetrahydropyrimidin-2ylamino)ethoxy]benzoylamino]-3-phenyl-propanoic acid ethyl ester hydrochloride;
3-[2-hydroxy-4-[2-(3,4,5,6-tetrahydropyrimidin-2ylamino)ethoxy]benzoylamino]-3-phenyl-propanoic acid;
3-[2-hydroxy-5-[3-(pyrimidin-2-ylamino)propoxy]benzoylamino]-3-phenyl-propanoic acid ethyl ester;
3-[2-hydroxy-5-[3-(pyrimidin-2-ylamino)propoxy]benzoylamino]-3-phenyl-propanoic acid;
3-[2-hydroxy-5-[3-(3,4,5,6-tetrahydropyrimidin-2-ylamino)propoxy]benzoylamino]-3-phenyl-propanoic acid ethyl ester hydrochloride;
3-[2-hydroxy-5-[3-(3,4,5,6-tetrahydropyrimidin-2-ylamino)propoxy]benzoylamino]-3-phenyl-propanoic acid;
2(S)-Benzyloxycarbonylamino-3-[2-hydroxy-4-[2-(pyrimidin-2-ylamino)ethoxy]-benzoylamino]propionic acid ethyl ester hydrochloride;
2(S)-Benzyloxycarbonylamino-3-[2-hydroxy-4-[2-(pyrimidin-2-ylamino)ethoxy]-benzoylamino]propionic acid methyl ester;
2(S)-Benzyloxycarbonylamino-3-[2-hydroxy-4-[2-(pyrimidin-2-ylamino)ethoxy]-benzoylamino]propionic acid;
2(S)-Benzenesulfonylamino-3-[2-hydroxy-4-(2-methylpyridin-2-ylamino)-ethoxy]benzoylamino]propionic acid; or
2-Amino-3-[2-hydroxy-4-[2-(3,4,5,6-tetrahydropyrimidin-2-ylamino)ethoxy]-benzoylamino]propionic acid dihydrochloride or pharmaceutical salts thereof.

43. A pharmaceutical composition comprising a compound of claim 1 and at least one pharmaceutically acceptable carrier or excipient.

44. A method of treating a mammal afflicted with a condition that is selected from metastasis, neovascularization, angiogenesis, tumor growth, inflammation, restenosis, bone resorption, and adenovirus infection which comprises providing to the mammal a therapeutically effective amount of a compound of the formula: 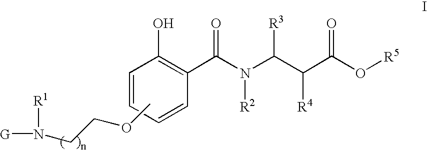
wherein
G is 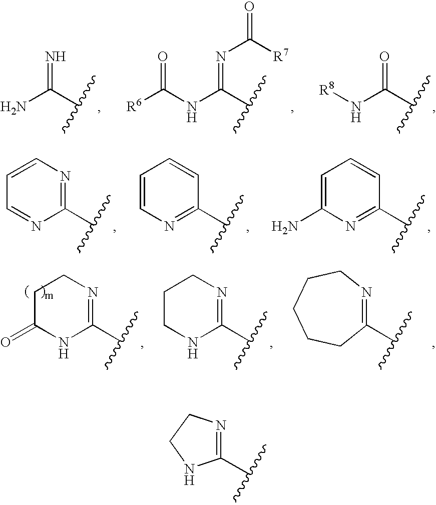
R¹ and R² are independently, hydrogen, alkyl of 1 to 6 carbon atoms, mono or bicyclic aralkyl of 6 to 10 carbon atoms, or heterocycloalkyl-alkyl comprised of a 5 to 10 membered mono or bicyclic heterocycloalkyl having 1 to 3 heteroatoms selected from S, N and O and an alkyl of 1 to 6 carbon atoms.
R³ is hydrogen, mono or bicyclic aryl of 6 to 10 carbon atoms, 5 to 10 membered mono or bicyclic heterocycloalkyl having 1 to 3 heteroatoms selected from S, N and O;
R⁴ is hydrogen, NHR⁹, OR⁹, NHCO₂R⁹, NHCONHR⁹, NHCOR⁹ or NHSO₂R⁹;
provided that R³ and R⁴ are not both hydrogen;
R⁵ is hydrogen, or alkyl of 1 to 6 carbon atoms, optionally substituted with a terminal group forming a prodrug;
R⁶ and R⁷ are hydrogen, alkyl of 1 to 6 carbon atoms, alkoxy of 1 to 6 carbon atoms, or aralkoxy of 6 to 10 carbon atoms;
R⁸ and R⁹ are independently hydrogen, trichloroalkylalkoxy, trifluoromethoxy phenyl, aralkenyl of 7 to 10 carbon atoms, alkyl of 1 to 10 carbon atoms, alkenyl of 2 to 10 carbons, alkynyl of 2 to 10 carbons, mono or polycycloalkyl of 3 to 10 carbon atoms, mono or bicyclic aryl of 6 to 10 carbon atoms, 6 to 10 membered mono or bicyclic heterocycloalkyl having 1 to 3 heteroatoms selected from S, N and O, mono or bicyclic aralkyl of 7 to 10 carbon atoms, or heterocycloalkyl-alkyl comprised of a 5 to 10 membered mono or bicyclic heterocycloalkyl having 1 to 3 heteroatoms selected from S, N and O and an alkyl of 1 to 6 carbon atoms;
n is an integer from 1 to 4;
m is 0 or 1;
or a pharmaceutical salt thereof.

45. The method of claim 44 wherein the integrin receptor is αᵥβ₃.

46. The method of claim 44 wherein the condition is cancer.

47. The method of claim 46 wherein the cancer is associated with at least one of metastasis, antiogenesis, neovascularization and tumor growth.

48. The method of claim 44 wherein the condition is benign tumor growth.

49. The method of claim 44 wherein the condition is neovascularization.

50. The method of claim 49 wherein neovascularization is associated with diabetic retinopathy, glaucoma, macular degeneration or blindness.

51. The method of claim 49 wherein neovascularization associated with rheumatoid arthritis.

52. The method of claim 44 wherein the condition is inflammation.

53. The method of claim 52 wherein the inflammation is associated with rheumatoid arthritis or psoriasis.

54. The method of claim 44 wherein the condition is restenosis.

55. The method of claim 54 wherein restenosis is associated with at least one of smooth muscle cell migration, smooth muscle cell proliferation, vascular endothelial cell migration and vascular endothelial cell proliferation.

56. The method of claim 44 wherein the condition is viral infection.

57. The method of claim 56 wherein the viral infection is caused by an adenovirus.

58. The method of claim 44 wherein the condition is characterized by bone resorption.

59. The method of claim 58 wherein bone resorption is associated with osteoporosis, Paget's disease, hypercalcemia, osteopenia, hyperparathyrodism, periarticular erosions, and periodontal disease.
